SEQUENCE LISTING (76 sequences)

SEQ ID NO: 1 - protein, 21 aa, synthetic construct
  REGION 1..21: note = CD8? signal/leader peptide (variant 1)
MALPVTALLLPLALLLHAARP

SEQ ID NO: 2 - protein, 19 aa, synthetic construct
  REGION 1..19: note = CD8? signal/leader peptide (variant 2)
MALPVTALLLPLALLLHAA

SEQ ID NO: 3 - protein, 11 aa, synthetic construct
  REGION 1..11: note = CD8? signal/leader peptide (variant 3)
MALPVTALLLP

SEQ ID NO: 4 - protein, 12 aa, synthetic construct
  REGION 1..12: note = CD8? signal/leader peptide (variant 4)
PVTALLLPLALL

SEQ ID NO: 5 - protein, 14 aa, synthetic construct
  REGION 1..14: note = CD8? signal/leader peptide (variant 5)
LLLPLALLLHAARP

SEQ ID NO: 6 - protein, 45 aa, synthetic construct
  REGION 1..45: note = CD8hinge
TTTPAPRPPTPAPTIASQPLSLRPEACRPAAGGAVHTRGLDFACD

SEQ ID NO: 7 - protein, 27 aa, synthetic construct
  REGION 1..27: note = CD28 Transmembrane (Tm) domain
FWVLVVVGGVLACYSLLVTVAFIIFWV

SEQ ID NO: 8 - protein, 235 aa, synthetic construct
  REGION 1..235: note = Surface glycoprotein CD8 alpha chain isoform 1 precursor(NP_001139345.1)
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 9 - protein, 42 aa, synthetic construct
  REGION 1..42: note = 4-1BB costimulatory domain
KRGRKKLLYIFKQPFMRPVQTTQEEDGCSCRFPEEEEGGCEL

SEQ ID NO: 10 - protein, 41 aa, synthetic construct
  REGION 1..41: note = CD28 costimulatory domain
RSKRSRLLHSDYMNMTPRRPGPTRKHYQPYAPPRDFAAYRS

SEQ ID NO: 11 - protein, 116 aa, synthetic construct
  REGION 1..116: note = CD2 costimulatory domain
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 12 - protein, 40 aa, synthetic construct
  REGION 1..40: note = CD4 costimulatory domain
CVRCRHRRRQAERMSQIKRLLSEKKTCQCPHRFQKTCSPI

SEQ ID NO: 13 - protein, 32 aa, synthetic construct
  REGION 1..32: note = CD8a costimulatory domain
LYCNHRNRRRVCKCPRPVVKSGDKPSLSARYV

SEQ ID NO: 14 - protein, 19 aa, synthetic construct
  REGION 1..19: note = CD8b costimulatory domain
HLCCRRRRARLRFMKQFYK

SEQ ID NO: 15 - protein, 235 aa, synthetic construct
  REGION 1..235: note = LAT costimulatory domain
HCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 16 - protein, 112 aa, synthetic construct
  REGION 1..112: note = CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 22 aa, synthetic construct
  REGION 1..22: note = P2A peptide
GSGATNFSLLKQAGDVEENPGP

SEQ ID NO: 18 - protein, 20 aa, synthetic construct
  REGION 1..20: note = (GGGGS)4 linker
GGGGSGGGGSGGGGSGGGGS

SEQ ID NO: 19 - protein, 15 aa, synthetic construct
  REGION 1..15: note = (GGGGS)3 linker
GGGGSGGGGSGGGGS

SEQ ID NO: 20 - protein, 10 aa, synthetic construct
  REGION 1..10: note = (GGGGS)2 linker
GGGGSGGGGS

SEQ ID NO: 21 - protein, 5 aa, synthetic construct
  REGION 1..5: note = (GGGGS)1 linker
GGGGS

SEQ ID NO: 22 - protein, 308 aa, synthetic construct
  REGION 1..308: note = hCD34
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 23 - protein, 313 aa, synthetic construct
  REGION 1..313: note = TrhCD34
VGPFEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSP

SEQ ID NO: 24 - protein, 330 aa, synthetic construct
  REGION 1..330: note = P2A-hCD34
GSGATNFSLLKQAGDVEENPGPMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 25 - protein, 335 aa, synthetic construct
  REGION 1..335: note = P2A-TrhCD34
GSGATNFSLLKQAGDVEENPGPVGPFEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSP

SEQ ID NO: 26 - protein, 710 aa, synthetic construct
  REGION 1..710: note = Human-Herpes Simplex Virus-1 (HSV) - thymidine kinase (TK)
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGEGGGGGDLGGVKLPHLFGKRLVEARMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLLLDRHPIAVMLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 27 - protein, 120 aa, synthetic construct
  REGION 1..120: note = CD2 heavy chain variable region (1)
EVKLEESGAELVKPGASVKLSCRTSGFNIKDTYIHWVKQRPEQGLKWIGRIDPANGNTKYDPKFQDKATVTADTSSNTAYLQLSSLTSEDTAVYYCVTYAYDGNWYFDVWGAGTAVTVSS

SEQ ID NO: 28 - protein, 108 aa, synthetic construct
  REGION 1..108: note = CD2 light chain variable region (1)
DIKMTQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKR

SEQ ID NO: 29 - protein, 121 aa, synthetic construct
  REGION 1..121: note = CD2 heavy chain variable region (2)
EVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSS

SEQ ID NO: 30 - protein, 113 aa, synthetic construct
  REGION 1..113: note = CD2 light chain variable region (2)
DIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIER

SEQ ID NO: 31 - protein, 121 aa, synthetic construct
  REGION 1..121: note = CD3 heavy chain variable region (OKT 3)
GSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 32 - protein, 107 aa, synthetic construct
  REGION 1..107: note = CD3 light chain variable region (OKT 3)
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 33 - protein, 122 aa, synthetic construct
  REGION 1..122: note = CD3 heavy chain variable region (UCHT1)
EVQLVESGGGLVQPGGSLRLSCAASGYSFTGYTMNWVRQAPGKCLEWVALINPYKGVSTYNQKFKDRFTISVDKSKNTAYLQMNSLRAEDTAVYYCARSGYYGDSDWYFDVWGQGTLVTVSS

SEQ ID NO: 34 - protein, 107 aa, synthetic construct
  REGION 1..107: note = CD3 heavy chain variable region (UCHT1)
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIK

SEQ ID NO: 35 - protein, 116 aa, synthetic construct
  REGION 1..116: note = CD7 heavy chain variable region
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVS

SEQ ID NO: 36 - protein, 108 aa, synthetic construct
  REGION 1..108: note = CD7 light chain variable region
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKR

SEQ ID NO: 37 - protein, 119 aa, synthetic construct
  REGION 1..119: note = FLT3 heavy chain variable region
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGVGAHDAFDIWGQGTTVTVSS

SEQ ID NO: 38 - protein, 112 aa, synthetic construct
  REGION 1..112: note = FLT3 light chain variable region
DVVMTQSPLSLPVTPGEPASISCRSSQSLLHSNGNNYLDWYLQKPGQSPQLLIYLGSNRASGVPDRFSGSGSDTDFTLQISRVEAEDVGVYYCMQGTHPAISFGQGTRLEIK

SEQ ID NO: 39 - protein, 122 aa, synthetic construct
  REGION 1..122: note = FLT3 heavy chain variable region
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSS

SEQ ID NO: 40 - protein, 107 aa, synthetic construct
  REGION 1..107: note = FLT3 light chain variable region
DIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIK

SEQ ID NO: 41 - protein, 118 aa, synthetic construct
  REGION 1..118: note = FLT3 heavy chain variable region
EVQLVQSGAEVKKPGASVKVSCKASGYTFTSYYMHWARQAPGQGLEWMGIINPSGGSTSYAQKFQGRVTMTRDTSTSTVYMELSSLRSEDTAVYYCARVVAAAVADYWGQGTLVTVSS

SEQ ID NO: 42 - protein, 112 aa, synthetic construct
  REGION 1..112: note = FLT3 light chain variable region
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQSLQTPFTFGPGTKVDIK

SEQ ID NO: 43 - protein, 120 aa, synthetic construct
  REGION 1..120: note = CS1 heavy chain variable region
QVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSS

SEQ ID NO: 44 - protein, 107 aa, synthetic construct
  REGION 1..107: note = CS1 light chain variable region
DIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELK

SEQ ID NO: 45 - protein, 120 aa, synthetic construct
  REGION 1..120: note = CD33 heavy chain variable region
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSSG

SEQ ID NO: 46 - protein, 115 aa, synthetic construct
  REGION 1..115: note = CD33 light chain variable region
GSEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 47 - protein, 483 aa, synthetic construct
  REGION 1..483: note = CAR Amino Acid Sequence
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 484 aa, synthetic construct
  REGION 1..484: note = CAR Amino Acid Sequence
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 488 aa, synthetic construct
  REGION 1..488: note = CAR Amino Acid Sequence
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 489 aa, synthetic construct
  REGION 1..489: note = CAR Amino Acid Sequence
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 493 aa, synthetic construct
  REGION 1..493: note = CAR Amino Acid Sequence
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 494 aa, synthetic construct
  REGION 1..494: note = CAR Amino Acid Sequence
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 498 aa, synthetic construct
  REGION 1..498: note = CAR Amino Acid Sequence
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 614 aa, synthetic construct
  REGION 1..614: note = CAR Amino Acid Sequence
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 538 aa, synthetic construct
  REGION 1..538: note = CAR Amino Acid Sequence
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSTPAPRPPTPAPTIASQPLSLRPEACRPAAGGAVHTRGLDFACDFWVLVVVGGVLACYSLLVTVAFIIFWVCVRCRHRRRQAERMSQIKRLLSEKKTCQCPHRFQKTCSPIRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 733 aa, synthetic construct
  REGION 1..733: note = CAR Amino Acid Sequence
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSTPAPRPPTPAPTIASQPLSLRPEACRPAAGGAVHTRGLDFACDFWVLVVVGGVLACYSLLVTVAFIIFWVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 733 aa, synthetic construct
  REGION 1..733: note = CAR Amino Acid Sequence
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 58 - protein, 499 aa, synthetic construct
  REGION 1..499: note = CAR Amino Acid Sequence
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 621 aa, synthetic construct
  REGION 1..621: note = CAR Amino Acid Sequence
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSPRASTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 477 aa, synthetic construct
  REGION 1..477: note = CAR Amino Acid Sequence
MALPVTALLLPLALLLHAARPDIKMTQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSEVKLEESGAELVKPGASVKLSCRTSGFNIKDTYIHWVKQRPEQGLKWIGRIDPANGNTKYDPKFQDKATVTADTSSNTAYLQLSSLTSEDTAVYYCVTYAYDGNWYFDVWGAGTAVTVSSTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 478 aa, synthetic construct
  REGION 1..478: note = CAR Amino Acid Sequence
MALPVTALLLPLALLLHAARPDIKMTQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSEVKLEESGAELVKPGASVKLSCRTSGFNIKDTYIHWVKQRPEQGLKWIGRIDPANGNTKYDPKFQDKATVTADTSSNTAYLQLSSLTSEDTAVYYCVTYAYDGNWYFDVWGAGTAVTVSSTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 482 aa, synthetic construct
  REGION 1..482: note = CAR Amino Acid Sequence
MALPVTALLLPLALLLHAARPDIKMTQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSEVKLEESGAELVKPGASVKLSCRTSGFNIKDTYIHWVKQRPEQGLKWIGRIDPANGNTKYDPKFQDKATVTADTSSNTAYLQLSSLTSEDTAVYYCVTYAYDGNWYFDVWGAGTAVTVSSTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 483 aa, synthetic construct
  REGION 1..483: note = CAR Amino Acid Sequence
MALPVTALLLPLALLLHAARPDIKMTQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSEVKLEESGAELVKPGASVKLSCRTSGFNIKDTYIHWVKQRPEQGLKWIGRIDPANGNTKYDPKFQDKATVTADTSSNTAYLQLSSLTSEDTAVYYCVTYAYDGNWYFDVWGAGTAVTVSSTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 487 aa, synthetic construct
  REGION 1..487: note = CAR Amino Acid Sequence
MALPVTALLLPLALLLHAARPDIKMTQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSGGGGSEVKLEESGAELVKPGASVKLSCRTSGFNIKDTYIHWVKQRPEQGLKWIGRIDPANGNTKYDPKFQDKATVTADTSSNTAYLQLSSLTSEDTAVYYCVTYAYDGNWYFDVWGAGTAVTVSSTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 488 aa, synthetic construct
  REGION 1..488: note = CAR Amino Acid Sequence
MALPVTALLLPLALLLHAARPDIKMTQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSGGGGSEVKLEESGAELVKPGASVKLSCRTSGFNIKDTYIHWVKQRPEQGLKWIGRIDPANGNTKYDPKFQDKATVTADTSSNTAYLQLSSLTSEDTAVYYCVTYAYDGNWYFDVWGAGTAVTVSSTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 492 aa, synthetic construct
  REGION 1..492: note = CAR Amino Acid Sequence
MALPVTALLLPLALLLHAARPDIKMTQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSGGGGSGGGGSEVKLEESGAELVKPGASVKLSCRTSGFNIKDTYIHWVKQRPEQGLKWIGRIDPANGNTKYDPKFQDKATVTADTSSNTAYLQLSSLTSEDTAVYYCVTYAYDGNWYFDVWGAGTAVTVSSTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 493 aa, synthetic construct
  REGION 1..493: note = CAR Amino Acid Sequence
MALPVTALLLPLALLLHAARPDIKMTQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSGGGGSGGGGSEVKLEESGAELVKPGASVKLSCRTSGFNIKDTYIHWVKQRPEQGLKWIGRIDPANGNTKYDPKFQDKATVTADTSSNTAYLQLSSLTSEDTAVYYCVTYAYDGNWYFDVWGAGTAVTVSSTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 608 aa, synthetic construct
  REGION 1..608: note = CAR Amino Acid Sequence
MALPVTALLLPLALLLHAARPDIKMTQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRGGGGSGGGGSGGGGSGGGGSEVKLEESGAELVKPGASVKLSCRTSGFNIKDTYIHWVKQRPEQGLKWIGRIDPANGNTKYDPKFQDKATVTADTSSNTAYLQLSSLTSEDTAVYYCVTYAYDGNWYFDVWGAGTAVTVSSTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 89 aa, synthetic construct
  REGION 1..89: note = CD132
FWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 70 - protein, 593 aa, synthetic construct
  REGION 1..593: note = CAR containing CD132 C' to CD3 zeta
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 71 - RNA, 108 nt, synthetic construct
  misc_feature 1..108: note = CD2 gRNA
  misc_feature 1: note = 2'OMe
  misc_feature 3: note = phosphorothioate
  misc_feature 5: note = phosphorothioate
  misc_feature 7: note = phosphorothioate
  misc_feature 101: note = 2'OMe
  misc_feature 103: note = phosphorothioate
  misc_feature 105: note = phosphorothioate
  misc_feature 107: note = phosphorothioate
  source 1..108: mol_type = other RNA
nancnangctgacaggctcgacacgttttagagctagaaatagcaagttaaaataaggctagtccgttatcaacttgaaaaagtggcaccgagtcggtgcntntntnt

SEQ ID NO: 72 - RNA, 108 nt, synthetic construct
  misc_feature 1..108: note = TRAC gRNA
  misc_feature 1: note = 2'OMe
  misc_feature 3: note = phosphorothioate
  misc_feature 5: note = phosphorothioate
  misc_feature 7: note = phosphorothioate
  misc_feature 101: note = 2'OMe
  misc_feature 103: note = phosphorothioate
  misc_feature 105: note = phosphorothioate
  misc_feature 107: note = phosphorothioate
  source 1..108: mol_type = other RNA
ngnangnaatcaaaatcggtgaatgttttagagctagaaatagcaagttaaaataaggctagtccgttatcaacttgaaaaagtggcaccgagtcggtgcntntntnt

SEQ ID NO: 73 - DNA, 22 bp, synthetic construct
  misc_feature 1..22: note = PCR Primer (CD2)(F, forward)
  source 1..22: mol_type = other DNA
accaaagatctcctggacttgt

SEQ ID NO: 74 - DNA, 20 bp, synthetic construct
  misc_feature 1..20: note = PCR Primer (CD2)(R, reverse)
  source 1..20: mol_type = other DNA
gcatgcctctgcctactgag

SEQ ID NO: 75 - DNA, 19 bp, synthetic construct
  misc_feature 1..19: note = PCR Primer (TRAC)(F, forward)
  source 1..19: mol_type = other DNA
ggggcaaagagggaaatga

SEQ ID NO: 76 - DNA, 21 bp, synthetic construct
  misc_feature 1..21: note = PCR Primer (TRAC)(R, reverse)
  source 1..21: mol_type = other DNA
gtcagatttgttgctccaggc

(19) United States
(12) Patent Application Publication
(10) Pub. No.: US 2024/0033357 A1
(43) Pub. Date: Feb. 1, 2024

(54) CHIMERIC ANTIGEN RECEPTORS AND USES THEREOF

(71) Applicant: WUGEN, Inc., St. Louis, MO (US)

(72) Inventors: You W. ZHOU, St. Louis, MO (US); Matthew COOPER, St. Louis, MO (US); James P. WHITE, St. Louis, MO (US)

(21) Appl. No.: 18/484,253

(22) Filed: Oct. 10, 2023

Related U.S. Application Data
(63) Continuation of application No. PCT/US2022/071692, filed on Apr. 13, 2022.
(60) Provisional application No. 63/174,321, filed on Apr. 13, 2021.

(51) Int. Cl. A61K 39/00; C07K 14/725; C07K 14/73; C07K 14/705; C07K 16/28; A61P 35/00

(52) U.S. Cl. CPC A61K 39/4631; C07K 14/7051; C07K 14/70514; C07K 14/70517; C07K 16/2806; A61K 39/4611; A61K 39/4613; A61K 39/464411; A61P 35/00; C07K 2319/03; C07K 2319/74

(57) ABSTRACT

The disclosure relates to chimeric antigen receptors and immune effector cells bearing chimeric antigen receptors (CARs). Disclosed herein are improved CARs that are able to recruit Lck and/or and PLCγ without the need to express CD2, thereby providing efficient CAR function without the risk of CD2-induced fratricide; optionally, the CARs also comprise CD132. Further disclosed herein are improved CARs specific for CD2. Further disclosed herein are improved immune effector cells bearing such improved CARs, therapeutic compositions comprising such improved immune effector cells, and methods for treating cancer using such improved immune effector cells and/or therapeutic compositions.

### CROSS REFERENCE TO RELATED APPLICATIONS

This application is a bypass continuation of International Application No. PCT/US2022/071692, filed Apr. 13, 2022, which claims priority to, and the benefit of, U.S. Provisional Patent Application No. 63/174,321, filed Apr. 13, 2021, the entireties of each are incorporated herein by reference.

### INCORPORATION OF SEQUENCE LISTING

The sequence listing that is contained in the file named “WGN0021-201BC1-US,” which is 116 kilobytes as measured in Microsoft Windows operating system and was created on Oct. 10, 2023, is filed electronically herewith and incorporated herein by reference.

T cells, a type of lymphocyte, play a central role in cell-mediated immunity. They are distinguished from other lymphocytes, such as B cells and natural killer cells (NK cells), by the presence of a T-cell receptor (TCR) on the cell surface. T helper cells (T_{H}), also called CD4⁺ T or CD4 T cells, express CD4 glycoprotein on their surface. Helper T cells are activated when exposed to peptide antigens presented by MHC (major histocompatibility complex) class II molecules. Once activated, these cells proliferate rapidly and secrete cytokines that regulate immune responses. Cytotoxic T cells (T_{C}), also known as CD8⁺ T cells or CD8 T cells, express CD8 glycoprotein on the cell surface. The CD⁺ T cells are activated when exposed to peptide antigens presented by MHC class I molecules. Memory T cells, a subset of T cells, persist long term and respond to their cognate antigen, thus providing the immune system with “memory” against past infections and/or tumor cells. Gamma delta (γδ) T cells are the prototype of ‘unconventional’ T cells and represent a relatively small subset of T cells in peripheral blood. They are defined by expression of heterodimeric T-cell receptors (TCRs) composed of γ and δ chains. This sets them apart from the CD4⁺ helper T cells and CD8⁺ cytotoxic T cells. Viral-specific cytotoxic T lymphocytes are T cells with reactivity against viral antigens, notably Epstein—Barr virus (EBV) and cytomegalovirus (CMV).

T cells and other immune effector cells can be genetically modified to express chimeric antigen receptors (CARs), which are fusion proteins comprised of an antigen recognition moiety and T cell activation domains. T cells expressing CARs can recognize a specific protein, i.e., antigen on tumor cells. These T cells expressing CARs can be expanded in the laboratory prior to infusion into a patient.

Clinical trials have shown high response rates after anti-CD19 CAR infusion in patients with B cell malignancies, including diffuse large B cell lymphoma (DLBCL) and B cell-precursor acute lymphoblastic leukemia (ALL), resulting in two FDA approved therapies YesCAR-Ta™ (axicabtagene ciloleucel, Kite Pharma/Gilead) and Kymriah™ (tisagenlecleucel, Novartis). Despite these successes, the development of CAR-T cell therapy against T cell malignancies has proven problematic, in part due to the shared expression of target antigens between malignant T cells and effector T cells. Among the most general challenges are: (1) the antigen target(s) for the CAR(s); (2) CAR design; and (3) tumor heterogeneity, particularly the variance in the surface expression of tumor antigens. The design of a CAR, for example the identity and location of the elements that make up its antigen-binding domain, the transmembrane and hinge domains, and the costimulatory and effector domains, can greatly affect the efficacy and persistence of CAR-bearing immune effector cells, and ultimately, their efficaciousness in the treatment of diseases.

Therefore, there remains a need for improved chimeric antigen receptor (CAR)-based immunotherapies, for more effective, safe, and efficient targeting of cancers, including T-cell associated malignancies.

To address the issue of tumor heterogeneity, particularly the variance in the surface expression of tumor antigens, it would be beneficial to develop CARs that can target additional tumor antigens. CD2 is another tumor antigen that may be targeted by CARs.

Lck, also known as lymphocyte-specific protein tyrosine kinase, has been reported to be associated with cytoplasmic (intracellular) domains of CD4 and CD8 along with the CD2 or CD28, and is involved in the steps of TCR mediated T cell activation. Lck is tightly controlled by a series of kinases and phosphorylases. Studies with the human Jurkat cell line have shown that suppression of Lck expression results in inhibition of TCR signaling. These and other studies have established a role for Lck in the TCR signaling pathway. In TCR mediated T cell activation, CD2, among multiple TCR co-receptors, recruits Lck to initiate the TCR signaling pathway and the phosphorylation of PLCγ, which leads to T-cell proliferation and activation of effector programming However, in CAR-T cells the presence of CD2 has been shown to induce fratricide amongst CD2-targeting CAR-T cells.

There is, therefore, a need for CARs that are able to recruit Lck and PLCγ through alternative means to recruitment by CD2. And there remains a need for improved CARs targeting CD2 in general.

### BRIEF DESCRIPTION

Disclosed herein are chimeric antigen receptors (CARs)and immune effector cells bearing CARs with improved signaling domains. In some embodiments, certain dispositions of CD4 or LAT domains in the intracellular domain of a CAR can improve recruitment of Lck or Lck and PLCγ to the interior of the cell membrane, without the need to express CD2. Accordingly, disclosed herein are CARs that are better able to recruit Lck and PLCγ in the absence of CD2, thereby providing efficient CAR function without the risk of CD2-induced fratricide. Also disclosed herein are improved immune effector cells bearing such improved CARs, therapeutic compositions comprising such improved immune effector cells, and methods for treating cancer using such improved immune effector cells and/or therapeutic compositions.

Also disclosed herein are CARs that target CD2 on cancer cells.

In a first aspect, provided herein are CARs that are able to recruit Lck and PLCγ to the interior of the cellular membrane of an immune effector cell due to an intracellular CD4 domain in the CAR.

In some embodiments, the CARs comprise, from a proximal N-terminus to a distal C-terminus, an optional CD8a leader polypeptide, an antigen binding domain, a hinge, a transmembrane domain, a CD4 domain, at least one co-stimulatory domain, and a signaling domain.

In some embodiments, the CARs comprise, from a proximal N-terminus to a distal C-terminus: an extracellular domain comprising an optional CD8a leader polypeptide, an antigen binding domain and a hinge; a transmembrane domain; and an intracellular domain comprising a CD4 domain, at least one co-stimulatory domain, and a signaling domain.

In a second aspect, provided herein are CARs that are able to recruit PLCγ to the interior of the cellular membrane of an immune effector cell due to one or more intracellular LAT domain in the CAR. (LAT recruits GADS and SLP76, and the heterotrimer consisting of LAT+GADS+SLP76 acts as a scaffold to recruit PLCγ.)

In some embodiments, the CARs comprise, from a proximal N-terminus to a distal C-terminus, an optional CD8a leader polypeptide, an antigen binding domain, a hinge, a transmembrane domain, a LAT domain or a peptide bond or a peptide or polypeptide linker, at least one co-stimulatory domain, a signaling domain, and optionally a LAT domain, provided that at least one LAT domain is present.

In some embodiments, the CARs comprise, from a proximal N-terminus to a distal C-terminus, an extracellular domain comprising an optional CD8a leader polypeptide, an antigen binding domain, and a hinge; a transmembrane domain; and an intracellular domain comprising a LAT domain or a peptide bond or a peptide or polypeptide linker, at least one co-stimulatory domain, a signaling domain, and optionally a LAT domain, provided that at least one LAT domain is present.

In a third aspect, provided herein are CARs comprising antigen-recognition domains targeting CD2.

In some embodiments, the CARs comprise, from a proximal N-terminus to a distal C-terminus, an optional CD8a leader polypeptide, a CD2-specific antigen binding domain, a hinge, a transmembrane domain, at least one co-stimulatory domain, and a signaling domain.

In some embodiments, the CARs comprise, from a proximal N-terminus to a distal C-terminus, an extracellular domain comprising an optional CD8a leader polypeptide, a CD2-specific antigen binding domain, and a hinge; a transmembrane domain; and an intracellular domain, at least one co-stimulatory domain, and a signaling domain.

In a fourth aspect, provided herein are immune effector cells bearing one or more CARs according to the first and/or second and/or third aspect. In some embodiments, the CAR-bearing immune effector cell is a chimeric antigen receptor T-cell (CAR-T cell) or a chimeric antigen receptor natural killer cell (CAR-NK).

In some embodiments, the CAR-bearing immune effector cell has additional features that ameliorate fratricide, alloreactivity and/or graft-versus-host reactions. In some embodiments, the CAR-bearing immune effector cell further comprises a suicide gene. In some embodiments, CAR-bearing immune effector cell is deficient in at least one or more antigens to which the one or more CARs specifically binds. In some embodiments where the CAR-bearing immune effector cell is a CAR-T cell, T cell receptor mediated signaling is blocked in the CAR-T cell. In some embodiments, the CAR-T cell is deficient in a subunit of the T cell receptor complex.

In some embodiments, the CAR-bearing immune effector cell specifically binds at least one antigen expressed on a malignant T cell. In some embodiments, the CAR-bearing immune effector cell specifically binds at least one antigen expressed on a malignant plasma cell. In some embodiments, the CAR-bearing immune effector cell specifically binds at least one antigen expressed on a malignant B cell.

In a fifth aspect, provided herein is a therapeutic composition comprising a population of CAR-bearing immune effector cells according to the fourth aspect and at least one therapeutically acceptable diluent, carrier and/or adjuvant.

In a sixth aspect, the provided herein is a method for treatment of cancer in a patient comprising administering a population of CAR-bearing immune effector cells according to the fourth aspect of the invention or a therapeutic composition according to the fifth aspect of the invention to a cancer patient.

### BRIEF DESCRIPTION OF THE DRAWINGS

FIG. 1 shows a proposed pathway for recruitment of Lck to the inner cell membrane by a CAR intracellular domain, in which the addition of CD4 ICD to the membrane-proximal region of the CAR intracellular (signaling) domain enhances Lck, and via Lck, PLCγ, recruitment. Note that P2A-TrCD34 is translated as a separate protein and is not present in the CAR.

FIG. 2 shows a proposed pathway for recruitment of PLCγ to the inner cell membrane by a CAR intracellular domain, in which the addition of LAT to the membrane proximal or C′ region of the CAR intracellular (signaling) domain enhances PLCγ recruitment. Note that P2A-TrCD34 is translated as a separate protein and is not present in the CAR.

FIG. 3 shows a schematic of the CAR2 and control CAR19 constructs used in in vitro tumor cell killing and mouse tumor survival studies. Note that P2A-TrCD34 is translated as a separate protein and is not present in the CAR.

FIG. 4 shows a schematic of CAR-T cell design and testing.

FIG. 5 shows a timeline of CAR-T cell preparation.

FIG. 6 shows FACS analysis of the efficiency of multiplex CRISPR/Cas9 gene editing of CD2 and TRAC.

FIG. 7 shows the efficiency of multiplex CRISPR/Cas9 gene editing of CD2 and TRAC as a bar chart.

FIG. 8 shows the results of an in vitro killing assay by UCART2 (top line, round data points) or UCART19 cells cultured with [⁵¹Cr]-labeled HH cells.

FIG. 9 shows the results of an in vitro killing assay by UCART2 (top line, round data points) or UCART19 cells cultured with [⁵¹Cr]-labeled Jurkat cells.

FIG. 10 shows the results of an in vitro killing assay by UCART2 (top line, round data points) or UCART19 cells cultured with [⁵¹Cr]-labeled Molt-3 cells.

FIG. 11 shows a Kaplan Meier survival curve of mice treated with UCART19 and with UCART2.

FIG. 12 shows tumor burden in a mouse leukemia model as determined by BLI imaging (bottom line, square data points are UCART2).

FIG. 13 shows a schematic of UCART19 (CD2+) and UCART19ACD2 (CD2−) generation.

FIG. 14 shows that deletion of CD2 attenuated UCART19 anti-tumor activity (bottom line, square data points are UCART2). Tumor burden was determined by BLI imaging BLI images were normalized to a color gradient scale.

FIG. 15 shows Kaplan Meier survival curves for untreated mice, mice treated with UCART19ΔACD2, and mice treated with UCART19 (top line, square data points).

FIG. 16A shows by FACS analysis that UCAR2 incorporating CD4 or LAT have a higher portion of T_{SCM/N} and lower T_{cm/em}.

FIG. 16B shows by CD34+percentage of cells that UCAR2 incorporating CD4 or LAT have a higher portion of T_{SCM/N} and lower T_{cm/em}. In the drawing, the gray box encloses a set of largely T_{CM} and T_{EM} data points (only two—T8 and T9 —are T_{N/SCM} and none are T_{EFF}, and the dashed box encloses set of largely T_{N/SCM} and T_{ECC} data points (only two—T8 and T9—are TEM and none are T_{CM}).

FIG. 17 shows that CAR19 cells bearing CD132 are able to continuously expand after repeat challenges unlike the other 2 CARs lacking CD132.

### DETAILED DESCRIPTION

Provided herein is a chimeric antigen receptor (CAR) comprising an extracellular domain, a transmembrane domain, and an intracellular domain, the intracellular domain comprising a domain which increases phospholipase C gamma (PLCγ) recruitment and phosphorylation, a costimulatory domain and a signaling domain. In some embodiments, the domain which increases PLCγ recruitment and phosphorylation is a domain which enhances recruitment of lymphocyte-specific protein tyrosine kinase (Lck) to the inner cell membrane. In some embodiments, the domain which enhances recruitment of Lck is CD4 or a functional fragment thereof disposed adjacent to the transmembrane domain. domain which increases PLCγ recruitment and phosphorylation is linker for activation of T cells (LAT) or a functional fragment thereof disposed adjacent to the transmembrane domain distal to the signaling domain.

In a first aspect, the invention provides CARs that are able to recruit Lck and PLCγ to the interior of the cellular membrane of an immune effector cell due to an intracellular CD4 domain in the CAR. Lck, when recruited to the interior of the cellular membrane, initiates the CAR signaling process, and leads to phosphorylation of PLCγ, which initiates proliferation and activation of effector programming of the immune effector cell.

In some embodiments, the CARs comprise, from a proximal N-terminus to a distal C-terminus, an optional CD8a leader polypeptide, an antigen binding domain, a hinge, a transmembrane domain, a CD4 domain, at least one co-stimulatory domain, and a signaling domain.

In some embodiments, the CARs comprise, from a proximal N-terminus to a distal C-terminus: an extracellular domain comprising an optional CD8a leader polypeptide, an antigen binding domain and a hinge; a transmembrane domain; and an intracellular domain comprising a CD4 domain, at least one co-stimulatory domain, and a signaling domain.

In some embodiments of CARs according to this aspect, the CD4 domain confers upon an immune effector cell bearing the CAR enhanced Lck recruitment to the inner cell membrane when the CAR is present as part of an immune effector cell. Lck, when recruited to the interior of the cellular membrane, initiates the CAR signaling process. Lck is also responsible for initiating PLCγ phosphorylation, with consequent activation of the inositol phospholipid pathway and mobilization of intracellular Ca²⁺, leading to the induction of interleukin 2 (IL-2) synthesis and entry into proliferation.

In a second aspect, provided herein are CARs that are able to recruit PLCγ to the interior of the cellular membrane of an immune effector cell due to an intracellular LAT domain. (LAT recruits GADS and SLP76, and the heterotrimer consisting of LAT+GADS+SLP76 acts as a scaffold to recruit PLCγ.) PLCγ, when recruited to the interior of the cellular membrane, plays a role in proliferation and activation of effector programming of CAR-bearing immune effector cells.

In some embodiments, the CARs comprise, from a proximal N-terminus to a distal C-terminus: an optional CD8a leader polypeptide, an antigen binding domain, a hinge, a transmembrane domain, a LAT domain or a peptide bond or a peptide or polypeptide linker, at least one co-stimulatory domain, a signaling domain, and optionally, a LAT domain, provided that at least one LAT domain is present.

In some embodiments, the CARs comprise, from a proximal N-terminus to a distal C-terminus: an extracellular domain comprising an optional CD8a leader polypeptide, an antigen binding domain, and a hinge; a transmembrane domain; and an intracellular domain comprising a LAT domain or a peptide bond or a peptide or polypeptide linker, at least one co-stimulatory domain, a signaling domain, and optionally a LAT domain, provided that at least one LAT domain is present. In some embodiments, the LAT domain has the amino acid sequence according to SEQ ID NO:15.

In some embodiments of CARs according to this aspect, the LAT domain confers upon an immune effector cell bearing the CAR enhanced Lck and PLCγ recruitment to the inner cell membrane when the CAR is present as part of an immune effector cell.

In a third aspect, provided herein are CARs that specifically target CD2.

In some embodiments, the CARs comprise, from a proximal N-terminus to a distal C-terminus, an optional CD8a leader polypeptide, a CD2-specific antigen binding domain, a hinge, a transmembrane domain, at least one co-stimulatory domain, and a signaling domain.

In some embodiments, the CARs comprise, from a proximal N-terminus to a distal C-terminus, an extracellular domain comprising an optional CD8a leader polypeptide, a CD2-specific antigen binding domain and a hinge; a transmembrane domain; and an intracellular domain comprising at least one co-stimulatory domain, and a signaling domain.

The CD34 or TrCD34 is translated as a separate protein from the CAR and allows selection for cells containing the CAR through anti-CD34 antibodies or other binding agents attached to magnetic beads or a solid surface.

CARs according to the first aspect, the second aspect, and the third aspect of the invention may have the following further embodiments, which may be present in various combinations to form additional embodiments.

In some embodiments, CARs have a signal/leader peptide that guides the translocation of the extracellular domain of the CAR through the immune effector cell membrane to the cell surface. In some embodiments, the signal/leader peptide is a CD8a signal/leader peptide. In some embodiments, the CD8α signal/leader peptide has an amino acid sequence according to any of SEQ ID NOS: 1-5.

CARs provided herein have an antigen binding domain. In some embodiments, the antigen binding domain comprises a V_{H} chain, a peptide linker and a V_{L} chain. In some embodiments, the V_{H} chain, peptide linker and a V_{L} chain together comprise an antigen binding domain specific for CD2. In some embodiments, the V_{H} chain, peptide linker and a V_{L} chain together comprise an antigen binding domain specific for any of CD3, CD7, FLT3, CS1, and CD33.

In some embodiments, the peptide linker between the V_{H} and V_{L} chains has the amino acid sequence GGGGS₍₁₋₄₎. In some embodiments, the peptide linker between the V_{H} and V_{L} chains has an amino acid sequence according to any of SEQ ID NOS:18-21.

In some embodiments, the V_{H} chain has an amino acid sequence according to any of SEQ ID NOS: 27, 29, 31, 33, 35, 37, 39, 41, 43, and 45. In some embodiments, the V_{L} chain has an amino acid sequence according to any of SEQ ID NOS: 28, 30, 32, 34, 36, 38, 40, 42, 44, and 46.

In some embodiments, the CAR has at least one costimulatory domain selected from CD2, CD8a, CD8b CD28, ICOS, CD27, OX40, 4-1BB and CD4. In some embodiments, the CAR has at least one costimulatory domain selected from CD2, CD28, 4-1BB and CD4. In some embodiments, at least one costimulatory domain is CD2. In some embodiments, the CD2 domain has the amino acid sequence according to SEQ ID NO:11. In some embodiments, the CD8a domain has the amino acid sequence according to SEQ ID NO:13. In some embodiments, the CD8b domain has the amino acid sequence according to SEQ ID NO:14. In some embodiments, at least one costimulatory domain is CD28. In some embodiments, the CD28 domain has the amino acid sequence according to SEQ ID NO:10. In some embodiments, at least one costimulatory domain is 4-1BB. In some embodiments, the 4-1BB domain has the amino acid sequence according to SEQ ID NO:9. In some embodiments, at least one costimulatory domain is CD4. In some embodiments, the CD4 domain has the amino acid sequence according to SEQ ID NO: 12. In some embodiments, the CAR has one co-stimulatory domain In some embodiments, the CAR has two co-stimulatory domains. In some embodiments, the CAR has a plurality of co-stimulatory domains.

In some embodiments, the signaling domain is a CD3 zeta (CD3ζ) domain. In some embodiments, the CD3ζ domain has the amino acid sequence according to SEQ ID NO:16. In some embodiments, the signaling domain is a CD132 domain. In some embodiments, the CD132 domain has the amino acid sequence according to SEQ ID NO:69.

Also provided herein are chimeric antigen receptors (CARs; and transgenic T-cell receptors, TCRs) comprising polypeptides as disclosed herein, e.g. as disclosed in Tables 2, 3, 12 and 13, and immune effector cells expressing them. A CAR is a recombinant fusion protein comprising: 1) an extracellular ligand-binding domain, i.e., an antigen-recognition domain, 2) a hinge domain, 3) a transmembrane domain, and 4) a cytoplasmic signaling domain, 5) and optionally, a co-stimulatory domain.

Methods for CAR design, delivery and expression, and the manufacturing of clinical-grade CAR-T cell populations are known in the art. CAR designs are generally tailored to each cell type.

The extracellular ligand-binding domain of a chimeric antigen receptor recognizes and specifically binds an antigen, typically a surface-expressed antigen of a malignant cell. The extracellular ligand-binding domain specifically binds an antigen when, for example, it binds the antigen with an affinity constant or affinity of interaction (K_{D}) between about 0.1 pM to about 10 μM, or about 0.1 μM to about 1 μM, or about 0.1 pM to about 100 nM. Methods for determining the affinity of interaction are known in the art. An extracellular ligand-binding domain can also be said to specifically bind a first polymorphic variant of an antigen when it binds it selectively over a second polymorphic variant of the same antigen.

An extracellular ligand-binding domain suitable for use in a CAR may be any antigen-binding polypeptide, a wide variety of which are known in the art. In some instances, the extracellular ligand-binding domain is a single chain Fv (scFv). Other antibody based recognition domains (cAb VHH (camelid antibody variable domains) and humanized versions thereof, lgNAR VH (shark antibody variable domains) and humanized versions thereof, sdAb VH (single domain antibody variable domains) and “camelized” antibody variable domains are suitable for use. In some instances, T-cell receptor (TCR) based recognition domains such as single chain TCR (scTv, single chain two-domain TCR containing VaV(3) are also suitable for use. In some embodiments, the extracellular ligand-binding domain is constructed from a natural binding partner, or a functional fragment thereof, to a target antigen. For example, CARs in general may be constructed with a portion of the APRIL protein, targeting the ligand for the B-Cell Maturation Antigen (BCMA) and Transmembrane Activator and CAML Interactor (TACI), effectively co-targeting both BCMA and TACI for the treatment of multiple myeloma.

The targeted antigen to which the CAR binds via its extracellular ligand-binding domain (or antigen-binding domain) may be an antigen that is expressed on a malignant myeloid (AML) cell, T cell or other cell. Antigens expressed on a malignant myeloid (AML) cells include CD33, FLT3, CD123, and CLL-1. Antigens expressed on T cells include CD2, CD3, CD4, CDS, CD7, TCRα (TRAC), TCRβ, CD70 and CD1a. Antigens expressed on malignant plasma cells include BCMA, CS1, CD38, CD79A, CD79B, CD138, and CD19. Antigens expressed on malignant B cells include CD19, CD20, CD21, CD22, CD23, CD24, CD25, CD27, CD38, and CD45.

Typically, the extracellular ligand-binding domain is linked to the intracellular domain of the chimeric antigen receptor by a transmembrane (TM) domain. A peptide hinge connects the extracellular ligand-binding domain to the transmembrane domain. The hinge is generally any oligo- or polypeptide that functions to link the transmembrane domain to the extracellular ligand-binding domain In particular, the hinge is used to provide more flexibility and accessibility for the extracellular antigen binding domain. A hinge may comprise up to 300 amino acids, or 10 to 100 amino acids, or 25 to 50 amino acids. The hinge may be derived from all or parts of naturally-occurring molecules such as CD28, 4-1BB (CD137), OX-40 (CD134), CD3ζ, the T cell receptor α or β chain, CD45, CD4, CD5, CD8, CD8α, CD9, CD16, CD22, CD33, CD37, CD64, CD80, CD86, ICOS, CD154 or from all or parts of an antibody constant region. Alternatively, the hinge may be a synthetic sequence that corresponds to a naturally-occurring hinge sequence or the hinge region may be an entirely synthetic hinge sequence. In some embodiments, the hinge domain comprises a part of human CD8α, FcγRIIIα receptor, or IgGl, and have at least 80%, 90%, 95%, 97%, or 99% sequence identity thereto. In some embodiments, the CD8α hinge comprises the amino acid sequence according to SEQ ID NO:6.

The transmembrane domain traverses the cell membrane, anchors the CAR to the T cell surface, and connects the extracellular ligand-binding to the intracellular (cytoplasmic) signaling domain, thus impacting expression of the CAR on the T cell surface.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. In some embodiments, the transmembrane domain is a CD28 transmembrane domain comprising the amino acid sequence according to SEQ ID NO:7. Alternatively, the transmembrane domain can be synthetic and comprise predominantly hydrophobic amino acid residues (e.g., leucine and valine). In some cases, a triplet of phenylalanine, tryptophan and valine will be found at each end of a synthetic transmembrane domain. In some embodiments, the transmembrane domain is derived from the T-cell surface glycoprotein CD8 alpha chain isoform 1 precursor (NP_001139345.1) or CD28. A short oligo- or polypeptide linker, such as between 2 and 10 amino acids in length, may form the linkage between the transmembrane domain and the endoplasmic domain of the CAR. In some embodiments, the CAR has more than one transmembrane domain, which can be a repeat of the same transmembrane domain, or can be different transmembrane domains.

After antigen recognition, the cytoplasmic signaling domain transmits a signal to the immune effector cell, activating at least one of the normal effector functions of the immune effector cell. Effector function of a T cell, for example, may be cytolytic activity or helper activity including the secretion of cytokines. While usually the entire cytoplasmic signaling domain can be employed, in many cases it is not necessary to use the entire chain. To the extent that a truncated portion of the cytoplasmic signaling domain is used, such truncated portion may be used in place of the intact chain as long as it transduces the effector function.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD36δ, CD3γ, CD3ε, CD32 (FcγRIIA), DAP10, DAP12, CD79a, CD79b, FcγRIγ, FcγRIIIγ, FcεRIβ (FCERIB), and FcεRIγ (FCERIG).

First-generation CARs typically have the cytoplasmic signaling domain from the CD3 chain, which is the primary transmitter of signals from endogenous TCRs. Second-generation CARs add cytoplasmic signaling domains from various co-stimulatory protein receptors (e.g., CD28, 4-1BB, ICOS) to the cytoplasmic signaling domain of the CAR to provide additional signals to the T cell.

A costimulatory domain is derived from the intracellular signaling domains of costimulatory proteins that enhance cytokine production, proliferation, cytotoxicity, and/or persistence in vivo. Preclinical studies have indicated that the second generation of CAR designs improves the antitumor activity of T cells. More recent, third-generation, and later generation, CARs combine multiple costimulatory domains to further augment potency. T cells grafted with these CARs have demonstrated improved expansion, activation, persistence, and tumor-eradicating efficiency independent of costimulatory receptor/ligand interaction.

For example, the cytoplasmic signaling domain of the CAR can be designed to comprise the signaling domain (e.g., CD3ζ) or CD132, either by itself or combined with any other desired cytoplasmic domain(s) useful in the context of the CAR of the invention. For example, the cytoplasmic domain of the CAR can comprise a signaling domain (e.g., CD3ζ) chain portion and a costimulatory signaling region. The co-stimulatory signaling region refers to a portion of the CAR comprising the intracellular domain of a co-stimulatory molecule. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D.

In some embodiments, the cytoplasmic signaling domain is a CD3 zeta (CD3ζ) signaling domain. In some embodiments, the cytoplasmic signaling domain is a CD132 signaling domain. In some embodiments, the co-stimulatory domain comprises the cytoplasmic domain of CD28, 4-1BB, or a combination thereof. In some cases, the co-stimulatory signaling region contains 1, 2, 3, or 4 cytoplasmic domains of one or more intracellular signaling and/or co-stimulatory molecules.

The co-stimulatory signaling domain(s) may contain one or more mutations in the cytoplasmic domains of CD28 and/or 4-1BB that enhance signaling. In some embodiments, the disclosed CARs comprises a co-stimulatory signaling region comprising a mutated form of the cytoplasmic domain of CD28 with altered phosphorylation at Y206 and/or Y218. In some embodiments, the disclosed CAR comprises an attenuating mutation at Y206, which will reduce the activity of the CAR. In some embodiments, the disclosed CAR comprises an attenuating mutation at Y218, which will reduce expression of the CAR. Any amino acid residue, such as alanine or phenylalanine, can be substituted for the tyrosine to achieve attenuation. In some embodiments, the tyrosine at Y206 and/or Y218 is substituted with a phosphomimetic residue. In some embodiments, the disclosed CAR substitution of Y206 with a phosphomimetic residue, which will increase the activity of the CAR. In some embodiments, the disclosed CAR comprises substitution of Y218 with a phosphomimetic residue, which will increase expression of the CAR. For example, the phosphomimetic residue can be phosphotyrosine. In some embodiments, a CAR may contain a combination of phosphomimetic amino acids and substitution(s) with non-phosphorylatable amino acids in different residues of the same CAR. For instance, a CAR may contain an alanine or phenylalanine substitution in Y209 and/or Y191 plus a phosphomimetic substitution in Y206 and/or Y218.

In some embodiments, the disclosed CARs comprise one or more 4-1BB domains with mutations that enhance binding to specific TRAF proteins, such as TRAF1, TRAF2, TRAF3, TRAF4, TRAF5, TRAF6, or any combination thereof. In some cases, the 41BB mutation enhances TRAF1- and/or TRAF2-dependent proliferation and survival of the T-cell, e.g. through NF-kB. In some cases, the 4-1BB mutation enhances TRAF3-dependent antitumor efficacy, e.g. through IRF7/INFβ. Therefore, the disclosed CARs can comprise cytoplasmic domain(s) of 4-1BB having at least one mutation in these underlined sequences that enhance TRAF-binding and/or enhance NFκB signaling.

Also as disclosed herein, TRAF proteins can in some cases enhance CAR T cell function independent of NFκB and 4-1BB. For example, TRAF proteins can in some cases enhance CD28 co-stimulation in T cells. Therefore, also disclosed herein are immune effector cells co-expressing CARs with one or more TRAF proteins, such as TRAF1, TRAF2, TRAF3, TRAF4, TRAF5, TRAF6, or any combination thereof. In some cases, the CAR is any CAR that targets a tumor antigen. For example, first-generation CARs typically had the intracellular domain from the CD3 chain, while second-generation CARs added intracellular signaling domains from various costimulatory protein receptors (e.g., CD28, 4-1BB, ICOS) to the cytoplasmic signaling domain of the CAR to provide additional signals to the T cell. In some cases, the CAR is the disclosed CAR with enhanced 4-1BB activation.

In some embodiments, the nucleic acid encoding the CAR may optionally encode a peptide or polypeptide linker and a CD34 domain or a truncated CD34 (TrCD34) domain. This peptide or polypeptide linker and a CD34 domain or truncated CD34 (TrCD34) domain is translated as a separate protein and is, therefore, not part of the CAR polypeptide.

The separately translated CD34 domain or TrCD34 domain can be used for selection of CAR-bearing cells, e.g., by using an anti-CD34 antibody or other CD34 binding agent attached to a surface or to magnetic beads. In some embodiments, the CD34 domain has the amino acid sequence according to SEQ ID NO:22. In some embodiments, the TrCD34 domain has the amino acid sequence according to SEQ ID NO:23. In some embodiments, the polypeptide linker is P2A. In some embodiments, the P2A linker has the amino acid sequence according to SEQ ID NO:16. In some embodiments, the P2A linker and the CD34 domain are contiguous and have the amino acid sequence according to SEQ ID NO: 24. In some embodiments, the P2A linker and the TrCD34 domain are contiguous and have the amino acid sequence according to SEQ ID NO: 25.

Variations on CAR components may be advantageous, depending upon the type of cell in which the CAR is expressed.

For example, in NK cells, in some embodiments, the transmembrane domain can be a sequence associated with NKG2D, FcγRIIIa, NKp44, NKp30, NKp46, actKIR, NKG2C, or CD8α. In certain embodiments, the NK cell is a memory-like NK (ML-NK) or cytokine-induced memory-like NK (CIML-NK) cell and the TM domain is CD8 α. Certain TM domains that do not work well in NK cells generally may work in a subset; CD8α, for example, works in ML-NKs but not NK cells generally.

Similarly, in NK cells, in some embodiments, the intracellular signaling domain(s) can be any co-activating receptor(s) capable of functioning in an NK cell, such as, for example, CD28, CD137/41BB (TRAF, NFkB), CD134/0X40, CD278/ICOS, DNAM-1 (Y-motif), NKp80 (Y-motif), 2B4 (SLAMF):: ITSM, CRACC (CS1/SLAMF7):: ITSM, CD2 (Y-motifs, MAPK/Erk), CD27 (TRAF, NFkB), or integrins (e.g., multiple integrins).

Similarly, in NK cells, in some embodiments, an intracellular signaling domain can be a cytokine receptor capable of functioning in an NK cell. For example, a cytokine receptor can be a cytokine receptor associated with persistence, survival, or metabolism, such as IL-2/15Rbyc:: Jak1/3, STAT3/5, PI3K/mTOR, MAPK/ERK. As another example, a cytokine receptor can be a cytokine receptor associated with activation, such as IL-18R:: NFκB. As another example, a cytokine receptor can be a cytokine receptor associated with IFN-γ production, such as IL-12R:: STAT4. As another example, a cytokine receptor can be a cytokine receptor associated with cytotoxicity or persistence, such as IL-21R:: Jak3/Tyk2, or STAT3. As another example, an intracellular signaling domain can be a TM adapter, such as FceR1γ (ITAMx1), CD3ζ (ITAMx3), DAP12 (ITAMx1), or DAP10 (YxxM/YINM). As another example, CAR intracellular signaling domains (also known as endodomains) can be derived from costimulatory molecules from the CD28 family (such as CD28 and ICOS) or the tumor necrosis factor receptor (TNFR) family of genes (such as 4-1BB, OX40, or CD27). The TNFR family members signal through recruitment of TRAF proteins and are associated with cellular activation, differentiation and survival. Certain signaling domains that may not work well in all NK cells generally may work in a subset; CD28 or 4-1BB, for example, work in ML-NKs. As another example, the intracellular signaling domain can be CD132.

### ENUMERATED EMBODIMENTS

Accordingly, although other embodiments may be found throughout the disclosure, provided herein are the following embodiments:

Embodiment 1. A chimeric antigen receptor (CAR) comprising an extracellular domain, a transmembrane domain, and an intracellular domain, the intracellular domain comprising a domain which increases phospholipase C gamma (PLCγ) recruitment and phosphorylation, a costimulatory domain and a signaling domain.

Embodiment 2. The CAR according to claim 1, wherein the domain which increases phospholipase C gamma (PLCγ) recruitment and phosphorylation is a domain which enhances recruitment of lymphocyte-specific protein tyrosine kinase (Lck) to the inner cell membrane.

Embodiment 3. The CAR according to claim 2, wherein the domain which enhances recruitment of Lck is CD4 or a functional fragment thereof disposed adjacent to the transmembrane domain.

Embodiment 4. The CAR according to claim 2, wherein domain which increases phospholipase C gamma (PLCγ) recruitment and phosphorylation is linker for activation of T cells (LAT) or a functional fragment thereof disposed adjacent to the transmembrane domain and/or distal to the signaling domain.

Embodiment 5. The CAR according to claim 2 comprising, from a proximal N-terminus to a distal C-terminus:
an optional CD8a leader polypeptide;
an antigen binding domain;
a hinge;
a transmembrane domain;
a CD4 domain;
at least one co-stimulatory domain; and
a signaling domain.

Embodiment 6. The CAR according to claim 5 comprising, from a proximal N-terminus to a distal C-terminus:
an extracellular domain comprising an optional CD8a leader polypeptide, an antigen binding domain, and a hinge;
a transmembrane domain; and
an intracellular domain comprising a CD4 domain, at least one co-stimulatory domain, and a signaling domain.

Embodiment 7. The CAR according to any of claims 3, 5 and 6, wherein the CD4 costimulatory domain has the amino acid sequence according to SEQ ID NO:12.

Embodiment 8. The CAR according to claim 7, wherein the CAR has an amino acid sequence according to SEQ ID NO: 55.

Embodiment 9. The CAR according to claim 2 comprising, from a proximal N-terminus to a distal C-terminus:
an optional CD8a domain;
an antigen binding domain;
a hinge;
a transmembrane domain;
a LAT domain or a peptide bond or a peptide or polypeptide linker;
at least one co-stimulatory domain;
a signaling domain; and
optionally; a LAT domain;
provided that at least one LAT domain is present.

Embodiment 10. The CAR according to claim 9, comprising, from a proximal N-terminus to a distal C-terminus:
an extracellular domain comprising an optional CD8a leader polypeptide, an antigen binding domain, and a hinge;
a transmembrane domain;
an intracellular domain comprising a LAT domain or a peptide bond or a peptide or polypeptide linker, at least one co-stimulatory domain, a signaling domain, and optionally a LAT domain.

Embodiment 11. The CAR according to any of claims 4, 9 and 10, wherein at least one LAT costimulatory domain has the amino acid sequence according to SEQ ID NO:15.

Embodiment 12. The CAR according claim 11, wherein the CAR comprises the amino acid sequence according to any of SEQ ID NOS.: 56 and 57.

Embodiment 13. The CAR according to any of claims 1-6 and 9-10, wherein the antigen binding domain comprises a V_{H} chain, a peptide linker and a V_{L} chain.

Embodiment 14. The CAR according to claim 13, wherein the V_{H} chain comprises an amino acid sequence according to any of SEQ ID NOS: 27, 29, 31, 33, 35, 37, 39, 41, 43, and 45.

Embodiment 15. The CAR according to claim 13, wherein the V_{L} chain comprises an amino acid sequence according to any of SEQ ID NOS: 28, 30, 32, 34, 36, 38, 40, 42, 44, and 46.

Embodiment 16. The CAR according to claim 13, wherein the peptide linker between the V_{H} and V_{L} chains has the amino acid sequence GGGGS₍₁₋₄₎.

Embodiment 17. The CAR according to claim 16, wherein the peptide linker between the V_{H} and V_{L} chains has an amino acid sequence according to any of SEQ ID NOS:18-21.

Embodiment 18. A CAR comprising, from a proximal N-terminus to a distal C-terminus:
an optional CD8a leader polypeptide;
a CD2-specific antigen binding domain;
a hinge;
a transmembrane domain;
at least one co-stimulatory domain; and
a signaling domain;
with the proviso that the CAR does not have the sequence of SEQ ID NO:32 or SEQ ID NO:37

Embodiment 19. The CAR according to claim 18, comprising, from a proximal N-terminus to a distal C-terminus:
an extracellular domain comprising an optional CD8a leader polypeptide,
a CD2-specific antigen binding domain and a hinge;
a transmembrane domain; and
an intracellular domain comprising at least one co-stimulatory domain, and a signaling domain.

Embodiment 20. The CAR according to any of claims 18-19, wherein the. antigen binding domain comprises a V_{H} chain, a peptide linker and a V_{L} chain.

Embodiment 21. The CAR according to claim 20, wherein the V_{H} chain comprises an amino acid sequence according to any of SEQ ID NOS: 27, 29, 31, 33, 35, 37, 39, 41, 43, and 45.

Embodiment 22. The CAR according to claim 20, wherein the V_{L} chain comprises an amino acid sequence according to any of SEQ ID NOS: 28, 30, 32, 34, 36, 38, 40, 42, 44, and 46.

Embodiment 23. The CAR according to claim 20, wherein the CAR comprises the amino acid sequence according to any of SEQ ID NOS.: 27-47.

Embodiment 24. The CAR according to any of claims 1-6, 9-10, 14-49 and 21-23, wherein the CAR has one co-stimulatory domain.

Embodiment 25. The CAR according to any of claims 1-6, 9-10, 14-19 and 21-23, wherein the CAR has a plurality of co-stimulatory domains.

Embodiment 26. The CAR according to any of claims 1-6, 940, 14-19 and 21-23, wherein the CAR has two co-stimulatory domains.

Embodiment 27. The CAR according to any of claims 1-26, wherein the CAR has a CD132 signaling domain.

Embodiment 28. A CAR comprising, from a proximal N-terminus to a distal C-terminus:
an extracellular domain comprising an optional CD8a leader polypeptide, an antigen binding domain, and a hinge;
a transmembrane domain; and
an intracellular domain comprising, at least one co-stimulatory domain, and a CD132 signaling domain.

Embodiment 29. The CAR according to claim 28, wherein the CD132 co-stimulatory domain comprises an amino acid sequence according to SEQ ID NO.:69.

Embodiment 30. The CAR according to claim 29, comprising an amino acid sequence according to SEQ ID NO.:70.

Embodiment 31. The CAR according to any of claims 28-30, wherein the antigen binding domain comprises a V_{H} chain, a peptide linker and a V_{L} chain.

Embodiment 32. The CAR according to claim 31, wherein the V_{H} chain comprises an amino acid sequence according to any of SEQ ID NOS: 27, 29, 31, 33, 35, 37, 39, 41, 43, and 45.

Embodiment 33. The CAR according to claim 31, wherein the V_{L} chain comprises an amino acid sequence according to any of SEQ ID NOS: 28, 30, 32, 34, 36, 38, 42, 44, and 46.

Embodiment 34. The CAR according to claim 31, wherein the peptide linker between the V_{H} and V_{L} chains has the amino acid sequence GGGGS₍₁₋₄₎.

Embodiment 35. The CAR according to claim 34, wherein the peptide linker between the V_{H} and V_{L} chains has an amino acid sequence according to any of SEQ ID NOS:18-21.

Embodiment 36. An immune effector cell comprising at least one CAR(s) according to any of claims 1-35.

Embodiment 37. The immune effector cell according to claim 36 that is a T cell (CAR-T cell) or a natural killer cell (CAR-NK cell).

Embodiment 38. The immune effector cell according to claim 37 that is a CAR-T cell.

Embodiment 39. The CAR-T cell according to claim 38, wherein the CAR-T cell is deficient in a subunit of the T cell receptor complex and/or is deficient in at least one or more antigens to which the one or more CARs specifically binds.

Embodiment 40. The CAR-T cell according to claim 39, wherein the subunit of the T cell receptor complex is selected from one or more of TCRα, TCRβ, TCRδ, TCRγ, CD3ε, CD3γ, CD3δ, and an extracellular domain of CD3ζ.

Embodiment 41. The CAR-T cell according to claim 38, wherein the CAR-T cell is deficient in one or more antigens to which the one or more CARs specifically binds.

Embodiment 42. The CAR-T cell according to claim 41, wherein the CAR-T cell is deficient in CD2.

Embodiment 43. The CAR-T cell according to any of claims 38-42, wherein the CAR(s) specifically binds at least one antigen expressed on a malignant T cell.

Embodiment 44. The CAR-T cell according to claim 43, wherein the antigen expressed on the malignant T cell is selected from one or more of CD2, CD3, CD4, CD5, CD7, TRAC, CD70, CD1a, and TCRβ.

Embodiment 45. The CAR-T cell according to any of claims 38-42, wherein the CAR(s) specifically binds at least one antigen expressed on a malignant plasma cell.

Embodiment 46. The CAR-T cell according to claim 45, wherein the antigen expressed on the malignant plasma cell is selected from one or more of BCMA, CS1, CD38, CD79A, CD79B, CD138, and CD19.

Embodiment 47. The CAR-T cell according to any of claims 38-42, wherein the CAR(s) specifically binds at least one antigen expressed on a malignant B cell.

Embodiment 48. The CAR-T cell according to claim 47, wherein the antigen expressed on a malignant B cell is selected from one or more of CD19, CD20, CD21, CD22, CD23, CD24, CD25, CD27, CD38, and CD45.

Embodiment 49. The CAR-T cell according to claim 50, wherein the antigen expressed on a malignant B cell is selected from one or more of CD19 and CD20.

Embodiment 50. The CAR-T cell according to any of claims 38-42, wherein the CAR-T cell further comprises a suicide gene.

Embodiment 51. The CAR-T cell according to any of claims 38-42, wherein endogenous T cell receptor mediated signaling is blocked in the CAR-T cell.

Embodiment 52. The CAR-T cell according to claim 51, wherein the endogenous T cell receptor mediated signaling is blocked by insertion of the CAR into a locus involved in T cell signaling.

Embodiment 53. The CAR-T cell according to claim 52, wherein the locus is TRAC.

Embodiment 54. The CAR-T cell according to any of claims 38-42, wherein the CAR-T cells do not induce alloreactivity or graft-versus-host disease.

Embodiment 55. The CAR-T cell according to any of claims 38-42, wherein the CAR-T cells do not induce fratricide.

Embodiment 56. The immune effector cell according to claim 36 that is a CAR-NK cell.

Embodiment 57. The CAR-NK cell according to claim 56 wherein CAR-NK cell is selected from a ML cell, a memory-like NK cell, and a CIML cell.

Embodiment 58. The immune effector cell according to any of claims 36-57, wherein PD1 is deleted, and/or where expression of CD52 is suppressed.

Embodiment 59. A therapeutic composition comprising a population of CAR-bearing immune effector cells according to any of any of claims 36-58 and at least one therapeutically acceptable diluent, carrier and/or adjuvant.

Embodiment 60. A method for treatment of cancer in a patient comprising administering a population of CAR-bearing immune effector cells according to any of any of claims 36-58, or a therapeutic composition according to claim 59, to a cancer patient.

Embodiment 61. The method according to claim 60, wherein the immune effector cell or population of CAR-bearing immune effector cells is a CAR-T cell or a population of CAR-T cells, according to any of any of claims 38-55.

Embodiment 62. The method according to claim 60, wherein the immune effector cell or a population of CAR-bearing immune effector cells is a CAR-NK cell or a population of CAR-NK cells, according to any of any of claims 56 and 57.

Embodiment 63. The method according to any of claims 60-62, wherein the cancer is a hematologic malignancy.

Embodiment 64. The method according to claim 63, wherein the hematologic malignancy is multiple myeloma.

Embodiment 65. The method according to claim 63, wherein the hematologic malignancy is acute myeloid leukemia (AML).

Embodiment 66. The method according to claim 63, wherein the hematologic malignancy is a T-cell malignancy.

Embodiment 67. The method according to claim 66, wherein the T cell malignancy is T-cell acute lymphoblastic leukemia (T-ALL).

Embodiment 68. The method according to claim 66, wherein the T cell malignancy is non-Hodgkin's lymphoma.

Embodiment 69. The method according to claim 66, wherein the T cell malignancy is T-cell chronic lymphocytic leukemia (T-CLL).

Embodiment 70. The method according to claim 66, wherein the T cell malignancy is selected from T-cell acute lymphoblastic leukemia/lymphoma (T-ALL), human T-cell leukemia virus type 1-positive (HTLV-1 +) adult T-cell leukemia/lymphoma (ATL), T-cell prolymphocytic leukemia (T-PLL), Adult T-cell lymphoma/leukemia (HTLV-1 associated), Aggressive NK-cell leukemia, Anaplastic large-cell lymphoma (ALCL), ALK positive, Anaplastic large-cell lymphoma (ALCL), ALK negative, Angioimmunoblastic T-cell lymphoma (AITL), Breast implant-associated anaplastic large-cell lymphoma, Chronic lymphoproliferative disorder of NK cells, Extra nodal NK/T-cell lymphoma, nasal type, Enteropathy-type T-cell lymphoma, Follicular T-cell lymphoma, Hepatosplenic T-cell lymphoma, Indolent T-cell lymphoproliferative disorder of the GI tract, Monomorphic epitheliotrophic intestinal T-cell lymphoma, Mycosis fungoides, Nodal peripheral T-cell lymphoma with TFH phenotype, Peripheral T-cell lymphoma (PTCL), NOS, Primary cutaneous γδ T-cell lymphoma, Primary cutaneous CD8+ aggressive epidermotropic cytotoxic T-cell lymphoma, Primary cutaneous acral CD8+ T-cell lymphoma, Primary cutaneous CD4+ small/medium T-cell lymphoproliferative disorders [Primary cutaneous anaplastic large-cell lymphoma (C-ALCL), lymphoid papulosis], Sezary syndrome, Subcutaneous, panniculitis-like T-cell lymphoma, Systemic EBV+ T-cell lymphoma of childhood, and T-cell large granular lymphocytic leukemia (LGL).

Embodiment 71. The method according to claim 63, wherein the hematologic malignancy is a B-cell malignancy.

Embodiment 72. The method according to claim 71, wherein the B-cell malignancy is selected from diffuse large B cell lymphoma (DLBCL), chronic lymphocytic leukemia (CLL)/small lymphocytic lymphoma (SLL), and B cell-precursor acute lymphoblastic leukemia (ALL).

Embodiment 73. The method according to claim 63, wherein the hematologic malignancy is a plasma cell malignancy.

Embodiment 74. The method according to claim 73, wherein the plasma cell malignancy is selected from lymphoplasmacytic lymphoma, plasmacytoma and multiple myeloma.

### CAR Design and Construction

The chimeric antigen receptor (CAR) construct, which encodes the chimeric receptor can be prepared in conventional ways. Since, for the most part, natural sequences are employed, the natural genes are isolated and manipulated, as appropriate (e.g., when employing a Type II receptor, the immune signaling receptor component may have to be inverted), so as to allow for the proper joining of the various components. Thus, the nucleic acid sequences encoding for the N-terminal and C-terminal proteins of the chimeric receptor can be isolated by employing the polymerase chain reaction (PCR), using appropriate primers which result in deletion of the undesired portions of the gene. Alternatively, restriction digests of cloned genes can be used to generate the chimeric construct. In either case, the sequences can be selected to provide for restriction sites which are blunt-ended, or have complementary overlaps.

The various manipulations for preparing the chimeric construct can be carried out in vitro and in particular embodiments the chimeric construct is introduced into vectors for cloning and expression in an appropriate host using standard transformation or transfection methods. Thus, after each manipulation, the resulting construct from joining of the DNA sequences is cloned, the vector isolated, and the sequence screened to ensure that the sequence encodes the desired chimeric receptor. The sequence can be screened by restriction analysis, sequencing, or the like.

It is contemplated that the chimeric construct can be introduced into immune effector cells as naked DNA or in a suitable vector. Methods of stably transfecting immune effector cells by electroporation using naked DNA are known in the art. See, e.g., U.S. Pat. No. 6,410,319. Naked DNA generally refers to the DNA encoding a chimeric receptor of the present invention contained in a plasmid expression vector in proper orientation for expression. Advantageously, the use of naked DNA reduces the time required to produce immune effector cells expressing the chimeric receptor of the present invention.

Alternatively, a viral vector (e.g., a retroviral vector, adenoviral vector, adeno-associated viral vector, or lentiviral vector) can be used to introduce the chimeric construct into immune cell, e.g., T cells. Suitable vectors for use in accordance with the method of the present invention are non-replicating in the immune effector cells of the subject. A large number of vectors are known which are based on viruses, where the copy number of the virus maintained in the cell is low enough to maintain the viability of the cell. Illustrative vectors include the pFB-neo vectors (STRATAGENE™) as well as vectors based on HIV, SV40, EBV, HSV or BPV. Once it is established that the transfected or transduced immune effector cell is capable of expressing the chimeric receptor as a surface membrane protein with the desired regulation and at a desired level, it can be determined whether the chimeric receptor is functional in the host cell to provide for the desired signal induction (e.g., production of Rantes, Mip1-alpha, GM-CSF upon stimulation with the appropriate ligand).

Engineered CARs may be introduced into CAR-bearing immune effector cells using retroviruses, which efficiently and stably integrate a nucleic acid sequence encoding the chimeric antigen receptor into the target cell genome. Other methods known in the art include, but are not limited to, lentiviral transduction, transposon-based systems, direct RNA transfection, and CRISPR/Cas systems (e.g., type I, type II, or type III systems using a suitable Cas protein such Cas3, Cas4, Cas5, Cas5e (or CasD), Cash, Cas6e, Cas6f, Cas7, Cas8a1, Cas8a2, Cas8b, Cas8c, Cas9, Cas10, Cas1 Od, CasF, CasG, CasH, Csy 1, Csy2, Csy3, Csel (or CasA), Cse2 (or CasB), Cse3 (or CasE), Cse4 (or CasC), Csc 1, Csc2, Csa5, Csn2, Csm2, Csm3, Csm4, Csm5, Csm6, Cmr1, Cmr3, Cmr4, Cmr5, Cmr6, Csb1, Csb2, Csb3,Csx17, Csx14, Csx10, Csx16, CsaX, Csx3, Csz1, Csx15, Csf1, Csf2, Csf3, Csf4, and Cu1966, etc.). Zinc finger nucleases (ZFNs) and transcription activator-like effector nucleases (TALENs) may also be used. See, e.g., Shearer RF and Saunders DN, “Experimental design for stable genetic manipulation in mammalian cell lines: lentivirus and alternatives,” Genes Cells 2015 January; 20(1):1-10.

Editing strategies, such as CRISPR guided base editors (CBE), enable efficient genome editing with minimal DNA scission. The application for CBE in the context of engineered T cells includes silencing of TCR and HLA expression and, to prevent fratricide, target antigen expression. Editing is achieved by coupling inactivated Cas9 with nucleotide deaminase, guided by a target specific RNA sequence to convert C to T. Conversion efficiency can be enhanced by the inclusion of uracil DNA-glycosylase inhibitors. CBE can mediate gene silencing through the introduction of premature stop codons or splice site disruption resulting on non-sense mediated decay (NMD) of mRNA transcripts. Multiple iterations of CBE are known in the art. Common base editors include, without limitation, BE3, VQR-BE3, EQR-BE3, VRER-BE3, SaBE3, SaKKH-BE3, SaBE3-Gam, TAM, CRISPR X, ABE, and YEE-BE3.

Amino acid sequences for selected components which may be used to construct a CAR are disclosed below in Table 1 and Table 2, below.

TABLE 1
Amino acid sequences of different CAR components.

| | SEQ ID | |
|---|---|---|
| Functional domains | NO: | Amino acid sequence |
| CD8α signal/leader | SEQ ID | MALPVTALLLPLALLLHAARP |
| peptide (variant 1) | NO: 1 | |
| CD8α signal/leader | SEQ ID | MALPVTALLLPLALLLHAA |
| peptide (variant 2) | NO: 2 | |
| CD8α signal/leader | SEQ ID | MALPVTALLLP |
| peptide (variant 3) | NO: 3 | |
| CD8α signal/leader | SEQ ID | PVTALLLPLALL |
| peptide (variant 4) | NO: 4 | |
| CD8α signal/leader | SEQ ID | LLLPLALLLHAARP |
| peptide (variant 5) | NO: 5 | |
| CD8α hinge | SEQ ID | TTTPAPRPPTPAPTIASQPLSLRPEACRPAAGGAVHTR |
| | NO: 6 | GLDFACD |
| CD28 Transmembrane | SEQ ID | FWVLVVVGGVLACYSLLVTVAFIIFWV |
| (Tₘ) domain | NO: 7 | |
| Surface glycoprotein | SEQ ID | MALPVTALLLPLALLLHAARPSQFRVSPLDRTWNLG |
| CD8 alpha chain | NO: 8 | ETVELKCQVLLSNPTSGCSWLFQPRGAAASPTFLLYL |
| isoform 1 precursor | | SQNKPKAAEGLDTQRFSGKRLGDTFVLTLSDFRREN |
| (NP_001139345.1) | | EGYYFCSALSNSIMYFSHFVPVFLPAKPTTTPAPRPPT |
| | | PAPTIASQPLSLRPEACRPAAGGAVHTRGLDFACDIY |
| | | IWAPLAGTCGVLLLSLVITLYCNHRNRRRVCKCPRP |
| | | VVKSGDKPSLSARYV |
| 4-1BB costimulatory | SEQ ID | KRGRKKLLYIFKQPFMRPVQTTQEEDGCSCRFPEEEE |
| domain | NO: 9 | GGCEL |
| CD28 costimulatory | SEQ ID | RSKRSRLLHSDYMNMTPRRPGPTRKHYQPYAPPRDF |
| domain | NO: 10 | AAYRS |
| CD2 costimulatory | SEQ ID | KRKKQRSRRNDEELETRAHRVATEERGRKPHQIPAS |
| domain | NO: 11 | TPQNPATSQHPPPPPGHRSQAPSHRPPPPGHRVQHQP |
| | | QKRPPAPSGTQVHQQKGPPLPRPRVQPKPPHGAAEN |
| | | SLSPSSN |
| CD4 costimulatory | SEQ ID | CVRCRHRRRQAERMSQIKRLLSEKKTCQCPHRFQKT |
| domain | NO: 12 | CSPI |
| CD8a costimulatory | SEQ ID | LYCNHRNRRRVCKCPRPVVKSGDKPSLSARYV |
| domain | NO: 13 | |
| CD8b costimulatory | SEQ ID | HLCCRRRRARLRFMKQFYK |
| domain | NO: 14 | |
| LAT costimulatory | SEQ ID | HCHRLPGSYDSTSSDSLYPRGIQFKRPHTVAPWPPAY |
| domain | NO: 15 | PPVTSYPPLSQPDLLPIPRSPQPLGGSHRTPSSRRDSD |
| | | GANSVASYENEGASGIRGAQAGWGVWGPSWTRLTP |
| | | VSLPPEPACEDADEDEDDYHNPGYLVVLPDSTPATS |
| | | TAAPSAPALSTPGIRDSAFSMESIDDYVNVPESGESA |
| | | EASLDGSREYVNVSQELHPGAAKTEPAALSSQEAEE |
| | | VEEEGAPDYENLQELN |
| CD132 | SEQ ID | FWLERTMPRIPTLKNLEDLVTEYHGNFSAWSGVSKG |
| | NO: 69 | LAESLQPDYSERLCLVSEIPPKGGALGEGPGASPCNQ |
| | | HSPYWAPPCYTLKPET |
| CD3 zeta (ζ) | SEQ ID | RVKFSRSADAPAYKQGQNQLYNELNLGRREEYDVL |
| | NO: 16 | DKRRGRDPEMGGKPRRKNPQEGLYNELQKDKMAE |
| | | AYSEIGMKGERRRGKGHDGLYQGLSTATKDTYDAL |
| | | HMQALPPR |
| P2A peptide | SEQ ID | GSGATNFSLLKQAGDVEENPGP |
| | NO: 17 | |
| (GGGGS)₄ linker | SEQ ID | GGGGSGGGGSGGGGSGGGGS |
| | NO: 18 | |
| (GGGGS)₃ linker | SEQ ID | GGGGSGGGGSGGGGS |
| | NO: 19 | |
| (GGGGS)₂ linker | SEQ ID | GGGGSGGGGS |
| | NO: 20 | |
| (GGGGS)₁ linker | SEQ ID | GGGGS |
| | NO: 21 | |
| hCD34 | SEQ ID | MPRGWTALCLLSLLPSGFMSLDNNGTATPELPTQGT |
| | NO: 22 | FSNVSTNVSYQETTTPSTLGSTSLHPVSQHGNEATTN |
| | | ITETTVKFTSTSVITSVYGNTNSSVQSQTSVISTVFTTP |
| | | ANVSTPETTLKPSLSPGNVSDLSTTSTSLATSPTKPYT |
| | | SSSPILSDIKAEIKCSGIREVKLTQGICLEQNKTSSCAE |
| | | FKKDRGEGLARVLCGEEQADADAGAQVCSLLLAQS |
| | | EVRPQCLLLVLANRTEISSKLQLMKKHQSDLKKLGIL |
| | | DFTEQDVASHQSYSQKTLIALVTSGALLAVLGITGYF |
| | | LMNRRSWSPI |
| TrhCD34 | SEQ ID | VGPFEAMPRGWTALCLLSLLPSGFMSLDNNGTATPE |
| | NO. 23 | LPTQGTFSNVSTNVSYQETTTPSTLGSTSLHPVSQHG |
| | | NEATTNITETTVKFTSTSVITSVYGNTNSSVQSQTSVI |
| | | STVFTTPANVSTPETTLKPSLSPGNVSDLSTTSTSLAT |
| | | SPTKPYTSSSPILSDIKAEIKCSGIREVKLTQGICLEQN |
| | | KTSSCAEFKKDRGEGLARVLCGEEQADADAGAQVC |
| | | SLLLAQSEVRPQCLLLVLANRTEISSKLQLMKKHQS |
| | | DLKKLGILDFTEQDVASHQSYSQKTLIALVTSGALLA |
| | | VLGITGYFLMNRRSWSP |
| P2A-hCD34 | SEQ ID | GSGATNFSLLKQAGDVEENPGPMPRGWTALCLLSLL |
| | NO: 24 | PSGFMSLDNNGTATPELPTQGTFSNVSTNVSYQETTT |
| | | PSTLGSTSLHPVSQHGNEATTNITETTVKFTSTSVITS |
| | | VYGNTNSSVQSQTSVISTVFTTPANVSTPETTLKPSLS |
| | | PGNVSDLSTTSTSLATSPTKPYTSSSPILSDIKAEIKCS |
| | | GIREVKLTQGICLEQNKTSSCAEFKKDRGEGLARVL |
| | | CGEEQADADAGAQVCSLLLAQSEVRPQCLLLVLAN |
| | | RTEISSKLQLMKKHQSDLKKLGILDFTEQDVASHQS |
| | | YSQKTLIALVTSGALLAVLGITGYFLMNRRSWSPI |
| P2A-TrhCD34 | SEQ ID | GSGATNFSLLKQAGDVEENPGPVGPFEAMPRGWTA |
| | NO: 25 | LCLLSLLPSGFMSLDNNGTATPELPTQGTFSNVSTNV |
| | | SYQETTTPSTLGSTSLHPVSQHGNEATTNITETTVKFT |
| | | STSVITSVYGNTNSSVQSQTSVISTVFTTPANVSTPET |
| | | TLKPSLSPGNVSDLSTTSTSLATSPTKPYTSSSPILSDI |
| | | KAEIKCSGIREVKLTQGICLEQNKTSSCAEFKKDRGE |
| | | GLARVLCGEEQADADAGAQVCSLLLAQSEVRPQCL |
| | | LLVLANRTEISSKLQLMKKHQSDLKKLGILDFTEQD |
| | | VASHQSYSQKTLIALVTSGALLAVLGITGYFLMNRR |
| | | SWSP |
| Human-Herpes | SEQ ID | MPRGWTALCLLSLLPSGFMSLDNNGTATPELPTQGT |
| Simplex Virus-1 (HSV)- | NO: 26 | FSNVSTNVSYQETTTPSTLGSTSLHPVSQHGNEATTN |
| thymidine kinase | | ITETTVKFTSTSVITSVYGNTNSSVQSQTSVISTVFTTP |
| (TK) | | ANVSTPETTLKPSLSPGNVSDLSTTSTSLATSPTKPYT |
| | | SSSPILSDIKAEIKCSGIREVKLTQGICLEQNKTSSCAE |
| | | FKKDRGEGLARVLCGEEQADADAGAQVCSLLLAQS |
| | | EVRPQCLLLVLANRTEISSKLQLMKKHQSDLKKLGIL |
| | | DFTEQDVASHQSYSQKTLIALVTSGALLAVLGITGYF |
| | | LMNRRSWSPTGEGGGGGDLGGVKLPHLFGKRLVEA |
| | | RMASYPCHQHASAFDQAARSRGHSNRRTALRPRRQ |
| | | QEATEVRLEQKMPTLLRVYIDGPHGMGKTTTTQLLV |
| | | ALGSRDDIVYVPEPMTYWQVLGASETIANIYTTQHR |
| | | LDQGEISAGDAAVVMTSAQITMGMPYAVTDAVLAP |
| | | HVGGEAGSSHAPPPALTLLLDRHPIAVMLCYPAARY |
| | | LMGSMTPQAVLAFVALIPPTLPGTNIVLGALPEDRHI |
| | | DRLAKRQRPGERLDLAMLAAIRRVYGLLANTVRYL |
| | | QGGGSWWEDWGQLSGTAVPPQGAEPQSNAGPRPHI |
| | | GDTLFTLFRAPELLAPNGDLYNVFAWALDVLAKRL |
| | | RPMHVFILDYDQSPAGCRDALLQLTSGMVQTHVTTP |
| | | GSIPTICDLARTFAREMGEAN |

### Cell-Specific Variations

The CAR components and construction methods disclosed above are suitable for use in T cells and other immune effector cells, but are not exhaustive. Certain variations may be useful in subsets of cells, and are known in the art.

For example, in NK cells, the TM domain may be chosen or adapted from NKG2D, FcγRIIIa, NKp44, NKp30, NKp46, actKIR, NKG2C, or CD8α. NK cells also express a number of transmembrane adapters that are triggered via association with activating receptors, providing an NK cell specific signal enhancement. For example, the TM adapter can be chosen or adapted from FceR1γ (ITAMx1), CD3ζ (ITAMx3), DAP12 (ITAMx1), or DAP10 (YxxM/YINM). In certain embodiments, the TM domains and adapters may be paired, e.g.: NKG2D and DAP10, FcγRIIIa and CD3ζ or FceR1γ, NKp44 and DAP12, NKp30 and CD3ζ or FceR1γ, NKp46 and CD3ζ or FceR1γ, actKIR and DAP12, and NKG2C and DAP12.

In certain embodiments, in NK cells, the hinge domain may be chosen or adapted from, e.g., NKG2, TMα, or CD8.

In certain embodiments, in NK cells, the intracellular signaling and/or costimulatory domain may comprise one or more of: CD137/41BB (TRAF, NFkB), DNAM-1 (Y-motif), NKp80 (Y-motif), 2B4 (SLAMF):: ITSM, CRACC (CS1/SLAMF7):: ITSM, CD2 (Y-motifs, MAPK/Erk), CD27 (TRAF, NFkB); one or more integrins (e.g., multiple integrins); a cytokine receptor associated with persistence, survival, or metabolism, such as IL-2/15R:: Jak1/3, STAT3/5, PI3K/mTOR, and MAPK/ERK; a cytokine receptor associated with activation, such as IL-18R:: NFkB. a cytokine receptor associated with IFN-γ production, such as IL-12R:: STAT4; a cytokine receptor associated with cytotoxicity or persistence, such as IL-21R:: Jak3/Tyk2, or STAT3; and a TM adapter, as disclosed above. In some embodiments, the NK cell CAR comprises three signaling domains, a TM domain, and optionally, a TM adapter.

The choice of costimulatory domain may also depend on the phenotype or subtype of the NK cell; for example, in some experiments, 4-1BB may be effective as a costimulatory domain in memory-like (ML) NK cells (including CIMLs) but less efficacious in NK cells. Additionally, signaling domains that may be harnessed that are more selectively expressed in ML NK cells include DNAM-1, CD137, CD132, and CD2.

Table 2 below discloses the sequences of V_{H} and V_{L} domains which target the recited antigens. These sequences may be incorporated into CARs along with elements from Table 1 or as disclosed herein.

TABLE 2
Amino acid sequences of the variable heavy (V_{H}) and variable light (V_{L}) chains
of selected scFvs.

| | SEQ ID | |
|---|---|---|
| ScFv sequences | NO: | Amino acid sequence |
| CD2 heavy chain | SEQ ID | EVKLEESGAELVKPGASVKLSCRTSGFNIKDTYIH |
| variable region (1) | NO: 27 | WVKQRPEQGLKWIGRIDPANGNTKYDPKFQDKAT |
| | | VTADTSSNTAYLQLSSLTSEDTAVYYCVTYAYDG |
| | | NWYFDVWGAGTAVTVSS |
| CD2 light chain | SEQ ID | DIKMTQSPSSMYVSLGERVTITCKASQDINSFLSWF |
| variable region (1) | NO: 28 | QQKPGKSPKTLIYRANRLVDGVPSRFSGSGSGQDY |
| | | SLTISSLEYEDMEIYYCLQYDEFPYTFGGGTKLEM |
| | | KR |
| CD2 heavy chain | SEQ ID | EVQLEESGAELVRPGTSVKLSCKASGYTFTSYWM |
| variable region (2) | NO: 29 | HWIKQRPEQGLEWIGRIDPYDSETHYNEKFKDKAI |
| | | LSVDKSSSTAYIQLSSLTSDDSAVYYCSRRDAKYD |
| | | GYALDYWGQGTSVTVSS |
| CD2 light chain | SEQ ID | DIVMTQAAPSVPVTPGESVSISCRSSKTLLHSNGNT |
| variable region (2) | NO: 30 | YLYWFLQRPGQSPQVLIYRMSNLASGVPNRFSGSG |
| | | SETTFTLRISRVEAEDVGIYYCMQHLEYPYTFGGG |
| | | TKLEIER |
| CD3 heavy chain | SEQ ID | GSQVQLQQSGAELARPGASVKMSCKASGYTFTRY |
| variable region (OKT | NO: 31 | TMHWVKQRPGQGLEWIGYINPSRGYTNYNQKFK |
| 3) | | DKATLTTDKSSSTAYMQLSSLTSEDSAVYYCARY |
| | | YDDHYCLDYWGQGTTLTVSS |
| CD3 light chain | SEQ ID | QIVLTQSPAIMSASPGEKVTMTCSASSSVSYMNWY |
| variable region (OKT | NO: 32 | QQKSGTSPKRWIYDTSKLASGVPAHFRGSGSGTSY |
| 3) | | SLTISGMEAEDAATYYCQQWSSNPFTFGSGTKLEI |
| | | NR |
| CD3 heavy chain | SEQ ID | EVLVESGGGLVQPGGSLRLSCAASGYSFTGYTM |
| variable region | NO: 33 | NWVRQAPGKCLEWVALINPYKGVSTYNQKFKDR |
| (UCHT1) | | FTISVDKSKNTAYLQMNSLRAEDTAVYYCARSGY |
| | | YGDSDWYFDVWGQGTLVTVSS |
| CD3 heavy chain | SEQ ID | DIQMTQSPSSLSASVGDRVTITCRASQDIRNYLNW |
| variable region | NO: 34 | YQQKPGKAPKLLIYYTSRLESGVPSRFSGSGSGTD |
| (UCHT1) | | YTLTISSLQPEDFATYYCQQGNTLPWTFGCGTKVEI |
| | | K |
| CD7 heavy chain | SEQ ID | EVLVESGGGLVKPGGSLKLSCAASGLTFSSYAMS |
| variable region | NO: 35 | WVRQTPEKRLEWVASISSGGFTYYPDSVKGRFTIS |
| | | RDNARNILYLQMSSLRSEDTAMYYCARDEVRGYL |
| | | DVWGAGTTVTVS |
| CD7 light chain | SEQ ID | DIQMTQTTSSLSASLGDRVTISCSASQGISNYLNWY |
| variable region | NO: 36 | QQKPDGTVKLLIYYTSSLHSGVPSRFSGSGSGTDYS |
| | | LTISNLEPEDIATYYCQQYSKLPYTFGGGTKLEIKR |
| FLT3 heavy chain | SEQ ID | EVQLVQSGAEVKKPGASVKVSCKASGYTFTSYYM |
| variable region | NO: 37 | HWVRQAPGQGLEWMGIINPSGGSTSYAQKFQGRV |
| | | TMTRDTSTSTVYMELSSLRSEDTAVYYCARGVGA |
| | | HDAFDIWGQGTTVTVSS |
| FLT3 light chain | SEQ ID | DVVMTQSPLSLPVTPGEPASISCRSSQSLLHSNGNN |
| variable region | NO: 38 | YLDWYLQKPGQSPQLLIYLGSNRASGVPDRESGSG |
| | | SDTDFTLQISRVEAEDVGVYYCMQGTHPAISFGQG |
| | | TRLEIK |
| FLT3 heavy chain | SEQ ID | EVQLVQSGAEVKKPGSSVKVSCKASGGTFSSYAIS |
| variable region | NO: 39 | WVRQAPGQGLEWMGGIIPIFGTANYAQKFQGRVTI |
| | | TADKSTSTAYMELSSLRSEDTAVYYCATFALFGFR |
| | | EQAFDIWGQGTTVTVSS |
| FLT3 light chain | SEQ ID | DIQMTQSPSSLSASVGDRVTITCRASQSISSYLNWY |
| variable region | NO: 40 | QQKPGKAPKLLIYAASSLQSGVPSRFSGSGSGTDFT |
| | | LTISSLQPEDLATYYCQQSYSTPFTFGPGTKVDIK |
| FLT3 heavy chain | SEQ ID | EVQLVQSGAEVKKPGASVKVSCKASGYTFTSYYM |
| variable region | NO: 41 | HWARQAPGQGLEWMGIINPSGGSTSYAQKFQGRV |
| | | TMTRDTSTSTVYMELSSLRSEDTAVYYCARVVAA |
| | | AVADYWGQGTLVTVSS |
| FLT3 light chain | SEQ ID | DVVMTQSPLSLPVTPGEPASISCRSSQSLLHSNGYN |
| variable region | NO: 42 | YLDWYLQKPGQSPQLLIYLGSNRASGVPDRESGSG |
| | | SGTDFTLKISRVEAEDVGVYYCMQSLQTPFTFGPG |
| | | TKVDIK |
| CS1 heavy chain | SEQ ID | QVQLQQPGAELVRPGASVKLSCKASGYSFTTYWM |
| variable region | NO: 43 | NWVKQRPGQGLEWIGMIHPSDSETRLNQKFKDKA |
| | | TLTVDKSSSTAYMQLSSPTSEDSAVYYCARSTMIA |
| | | TRAMDYWGQGTSVTVSS |
| CS1 light chain | SEQ ID | DIVMTQSQKSMSTSVGDRVSITCKASQDVITGVAW |
| variable region | NO: 44 | YQQKPGQSPKLLIYSASYRYTGVPD |
| | | RFTGSGSGTDFTFTISNVQAEDLAVYYCQQHYSTP |
| | | LTFGAGTKLELK |
| CD33 heavy chain | SEQ ID | QVQLQQPGAEVVKPGASVKMSCKASGYTFTSYYI |
| variable region | NO: 45 | HWIKQTPGQGLEWVGVIYPGNDDISYNQKFQGKA |
| | | TLTADKSSTTAYMQLSSLTSEDSAVYYCAREVRLR |
| | | YFDVWGQGTTVTVSSSG |
| CD33 light chain | SEQ ID | GSEIVLTQSPGSLAVSPGERVTMSCKSSQSVFFSSS |
| variable region | NO: 46 | QKNYLAWYQQIPGQSPRLLIYWASTRESGVPDRFT |
| | | GSGSGTDFTLTISSVQPEDLAIYYCHQYLSSRTFGQ |
| | | GTKLEIKR |

Below in Table 3 are provided examples of chimeric antigen receptors which may be constructed and expressed in immune effector cells according to methods known in the art and disclosed herein.

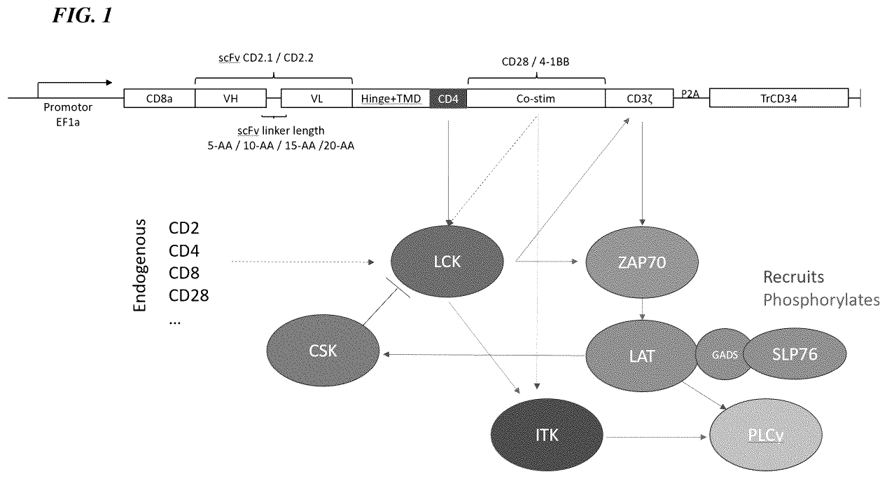

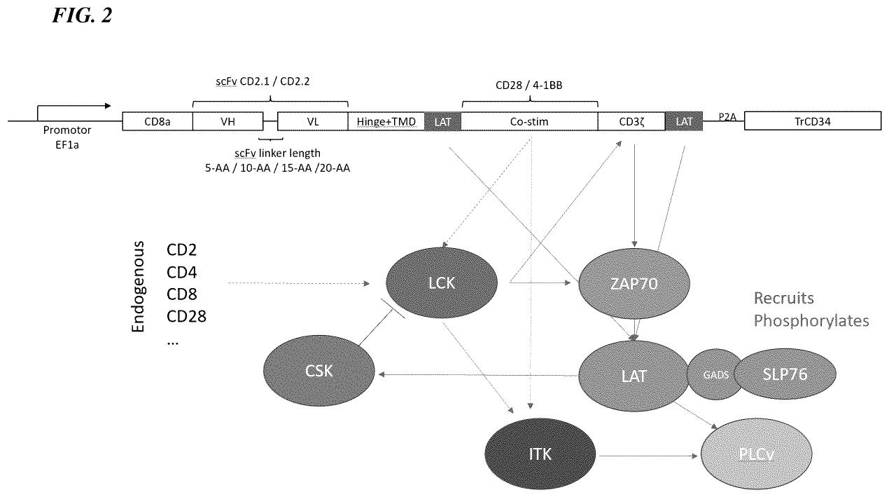

The CARs in Table 3 below are of the form:

|-[(leader)(V_{L})(linker)(V_{H}(hinge)(TMD)(costim)ₙ(effector)]-|,

wherein the leader sequence, VL, linker, hinge, transmembrane domain, costimulatory domain, optional second and optional third costimulatory domains (i.e., n is 1, 2, or 3), effector domain and optional P2a sequence and tag are disclosed above in Tables 1 and 2, or otherwise known in the art. Although FIGS. 1-3 show P2A-TrCD34, this reflects the fact that the DNA sequences encoding these are present in the nucleic acid sequences encoding the CARs. However, when the mRNA encoding the CAR is translated, the CAR portion distal to the P2A-TrCD34 and the P2A-TrCD34 (or P2A-CD34) are translated as separate polypeptides. Thus, the amino acid sequence of the CAR does not include P2A-TrCD34 (or P2A-CD34).

TABLE 3
Sequences of CAR Examples

| | SEQ | |
|---|---|---|
| CAR | ID | |
| Ex. | NO. | CAR Amino Acid Sequence |
| 1 | SEQ | MALPVTALLLPLALLLHAARPDIVMTQAAPSVPVTPGESVSISCRSSKT |
| | ID | LLHSNGNTYLYWFLQRPGQSPQVLIYRMSNLASGVPNRFSGSGSETTF |
| | NO: | TLRISRVEAEDVGIYYCMQHLEYPYTFGGGTKLEIERGGGGSEVQLEES |
| | 47 | GAELVRPGTSVKLSCKASGYTFTSYWMHWIKQRPEQGLEWIGRIDPY |
| | | DSETHYNEKFKDKAILSVDKSSSTAYIQLSSLTSDDSAVYYCSRRDAKY |
| | | DGYALDYWGQGTSVTVSSTPAPRPPTPAPTIASQPLSLRPEACRPAAGG |
| | | AVHTRGLDFACDFWVLVVVGGVLACYSLLVTVAFIIFWVRSKRSRLL |
| | | HSDYMNMTPRRPGPTRKHYQPYAPPRDFAAYRSRVKFSRSADAPAYK |
| | | QGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHM |
| | | QALPPR |
| 2 | SEQ | MALPVTALLLPLALLLHAARPDIVMTQAAPSVPVTPGESVSISCRSSKT |
| | ID | LLHSNGNTYLYWFLQRPGQSPQVLIYRMSNLASGVPNRFSGSGSETTF |
| | NO: | TLRISRVEAEDVGIYYCMQHLEYPYTFGGGTKLEIERGGGGSEVQLEES |
| | 48 | GAELVRPGTSVKLSCKASGYTFTSYWMHWIKQRPEQGLEWIGRIDPY |
| | | DSETHYNEKFKDKAILSVDKSSSTAYIQLSSLTSDDSAVYYCSRRDAKY |
| | | DGYALDYWGQGTSVTVSSTPAPRPPTPAPTIASQPLSLRPEACRPAAGG |
| | | AVHTRGLDFACDFWVLVVVGGVLACYSLLVTVAFIIFWVKRGRKKLL |
| | | YIFKQPFMRPVQTTQEEDGCSCRFPEEEEGGCELRVKFSRSADAPAYK |
| | | QGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHM |
| | | QALPPR |
| 3 | SEQ | MALPVTALLLPLALLLHAARPDIVMTQAAPSVPVTPGESVSISCRSSKT |
| | ID | LLHSNGNTYLYWFLQRPGQSPQVLIYRMSNLASGVPNRFSGSGSETTF |
| | NO: | TLRISRVEAEDVGIYYCMQHLEYPYTFGGGTKLEIERGGGGSGGGGSE |
| | 49 | VQLEESGAELVRPGTSVKLSCKASGYTFTSYWMHWIKQRPEQGLEWI |
| | | GRIDPYDSETHYNEKFKDKAILSVDKSSSTAYIQLSSLTSDDSAVYYCS |
| | | RRDAKYDGYALDYWGQGTSVTVSSTPAPRPPTPAPTIASQPLSLRPEA |
| | | CRPAAGGAVHTRGLDFACDFWVLVVVGGVLACYSLLVTVAFIIFWVR |
| | | SKRSRLLHSDYMNMTPRRPGPTRKHYQPYAPPRDFAAYRSRVKFSRSA |
| | | DAPAYKQGQNQLYNELNLGRREEYDVLDKRRGRDPEMGGKPRRKNP |
| | | QEGLYNELQKDKMAEAYSEIGMKGERRRGKGHDGLYQGLSTATKDT |
| | | YDALHMQALPPR |
| 4 | SEQ | MALPVTALLLPLALLLHAARPDIVMTQAAPSVPVTPGESVSISCRSSKT |
| | ID | LLHSNGNTYLYWFLQRPGQSPQVLIYRMSNLASGVPNRFSGSGSETTF |
| | NO: | TLRISRVEAEDVGIYYCMQHLEYPYTFGGGTKLEIERGGGGSGGGGSE |
| | 50 | VQLEESGAELVRPGTSVKLSCKASGYTFTSYWMHWIKQRPEQGLEWI |
| | | GRIDPYDSETHYNEKFKDKAILSVDKSSSTAYIQLSSLTSDDSAVYYCS |
| | | RRDAKYDGYALDYWGQGTSVTVSSTPAPRPPTPAPTIASQPLSLRPEA |
| | | CRPAAGGAVHTRGLDFACDFWVLVVVGGVLACYSLLVTVAFIIFWVK |
| | | RGRKKLLYIFKQPFMRPVQTTQEEDGCSCRFPEEEEGGCELRVKFSRSA |
| | | DAPAYKQGQNQLYNELNLGRREEYDVLDKRRGRDPEMGGKPRRKNP |
| | | QEGLYNELQKDKMAEAYSEIGMKGERRRGKGHDGLYQGLSTATKDT |
| | | YDALHMQALPPR |
| 5 | SEQ | MALPVTALLLPLALLLHAARPDIVMTQAAPSVPVTPGESVSISCRSSKT |
| | ID | LLHSNGNTYLYWFLQRPGQSPQVLIYRMSNLASGVPNRFSGSGSETTF |
| | NO: | TLRISRVEAEDVGIYYCMQHLEYPYTFGGGTKLEIERGGGGSGGGGSG |
| | 51 | GGGSEVQLEESGAELVRPGTSVKLSCKASGYTFTSYWMHWIKQRPEQ |
| | | GLEWIGRIDPYDSETHYNEKFKDKAILSVDKSSSTAYIQLSSLTSDDSA |
| | | VYYCSRRDAKYDGYALDYWGQGTSVTVSSTPAPRPPTPAPTIASQPLS |
| | | LRPEACRPAAGGAVHTRGLDFACDFWVLVVVGGVLACYSLLVTVAFI |
| | | IFWVRSKRSRLLHSDYMNMTPRRPGPTRKHYQPYAPPRDFAAYRSRV |
| | | KFSRSADAPAYKQGQNQLYNELNLGRREEYDVLDKRRGRDPEMGGK |
| | | PRRKNPQEGLYNELQKDKMAEAYSEIGMKGERRRGKGHDGLYQGLS |
| | | TATKDTYDALHMQALPPR |
| 6 | SEQ | MALPVTALLLPLALLLHAARPDIVMTQAAPSVPVTPGESVSISCRSSKT |
| | ID | LLHSNGNTYLYWFLQRPGQSPQVLIYRMSNLASGVPNRFSGSGSETTF |
| | NO: | TLRISRVEAEDVGIYYCMQHLEYPYTFGGGTKLEIERGGGGSGGGGSG |
| | 52 | GGGSEVQLEESGAELVRPGTSVKLSCKASGYTFTSYWMHWIKQRPEQ |
| | | GLEWIGRIDPYDSETHYNEKFKDKAILSVDKSSSTAYIQLSSLTSDDSA |
| | | VYYCSRRDAKYDGYALDYWGQGTSVTVSSTPAPRPPTPAPTIASQPLS |
| | | LRPEACRPAAGGAVHTRGLDFACDFWVLVVVGGVLACYSLLVTVAFI |
| | | IFWVKRGRKKLLYIFKQPFMRPVQTTQEEDGCSCRFPEEEEGGCELRV |
| | | KFSRSADAPAYKQGQNQLYNELNLGRREEYDVLDKRRGRDPEMGGK |
| | | PRRKNPQEGLYNELQKDKMAEAYSEIGMKGERRRGKGHDGLYQGLS |
| | | TATKDTYDALHMQALPPR |
| 7 | SEQ | MALPVTALLLPLALLLHAARPDIVMTQAAPSVPVTPGESVSISCRSSKT |
| | ID | LLHSNGNTYLYWFLQRPGQSPQVLIYRMSNLASGVPNRFSGSGSETTF |
| | NO: | TLRISRVEAEDVGIYYCMQHLEYPYTFGGGTKLEIERGGGGSGGGGSG |
| | 53 | GGGSGGGGSEVQLEESGAELVRPGTSVKLSCKASGYTFTSYWMHWIK |
| | | QRPEQGLEWIGRIDPYDSETHYNEKFKDKAILSVDKSSSTAYIQLSSLTS |
| | | DDSAVYYCSRRDAKYDGYALDYWGQGTSVTVSSTPAPRPPTPAPTIAS |
| | | QPLSLRPEACRPAAGGAVHTRGLDFACDFWVLVVVGGVLACYSLLVT |
| | | VAFIIFWVRSKRSRLLHSDYMNMTPRRPGPTRKHYQPYAPPRDFAAYR |
| | | SRVKFSRSADAPAYKQGQNQLYNELNLGRREEYDVLDKRRGRDPEM |
| | | GGKPRRKNPQEGLYNELQKDKMAEAYSEIGMKGERRRGKGHDGLYQ |
| | | GLSTATKDTYDALHMQALPPR |
| 8 | SEQ | MALPVTALLLPLALLLHAARPDIVMTQAAPSVPVTPGESVSISCRSSKT |
| | ID | LLHSNGNTYLYWFLQRPGQSPQVLIYRMSNLASGVPNRFSGSGSETTF |
| | NO: | TLRISRVEAEDVGIYYCMQHLEYPYTFGGGTKLEIERGGGGSGGGGSG |
| | 54 | GGGSGGGGSEVQLEESGAELVRPGTSVKLSCKASGYTFTSYWMHWIK |
| | | QRPEQGLEWIGRIDPYDSETHYNEKFKDKAILSVDKSSSTAYIQLSSLTS |
| | | DDSAVYYCSRRDAKYDGYALDYWGQGTSVTVSSTPAPRPPTPAPTIAS |
| | | QPLSLRPEACRPAAGGAVHTRGLDFACDFWVLVVVGGVLACYSLLVT |
| | | VAFIIFWVRSKRSRLLHSDYMNMTPRRPGPTRKHYQPYAPPRDFAAYR |
| | | SKRKKQRSRRNDEELETRAHRVATEERGRKPHQIPASTPQNPATSQHP |
| | | PPPPGHRSQAPSHRPPPPGHRVQHQPQKRPPAPSGTQVHQQKGPPLPRP |
| | | RVQPKPPHGAAENSLSPSSNRVKFSRSADAPAYKQGQNQLYNELNLG |
| | | RREEYDVLDKRRGRDPEMGGKPRRKNPQEGLYNELQKDKMAEAYSEI |
| | | GMKGERRRGKGHDGLYQGLSTATKDTYDALHMQALPPR |
| 9 | SEQ | MALPVTALLLPLALLLHAARPDIVMTQAAPSVPVTPGESVSISCRSSKT |
| | ID | LLHSNGNTYLYWFLQRPGQSPQVLIYRMSNLASGVPNRFSGSGSETTF |
| | NO: | TLRISRVEAEDVGIYYCMQHLEYPYTFGGGTKLEIERGGGGSGGGGSG |
| | 55 | GGGSGGGGSEVQLEESGAELVRPGTSVKLSCKASGYTFTSYWMHWIK |
| | | QRPEQGLEWIGRIDPYDSETHYNEKFKDKAILSVDKSSSTAYIQLSSLTS |
| | | DDSAVYYCSRRDAKYDGYALDYWGQGTSVTVSSTPAPRPPTPAPTIAS |
| | | QPLSLRPEACRPAAGGAVHTRGLDFACDFWVLVVVGGVLACYSLLVT |
| | | VAFIIFWVCVRCRHRRRQAERMSQIKRLLSEKKTCQCPHRFQKTCSPIR |
| | | SKRSRLLHSDYMNMTPRRPGPTRKHYQPYAPPRDFAAYRSRVKFSRSA |
| | | DAPAYKQGQNQLYNELNLGRREEYDVLDKRRGRDPEMGGKPRRKNP |
| | | QEGLYNELQKDKMAEAYSEIGMKGERRRGKGHDGLYQGLSTATKDT |
| | | YDALHMQALPPR |
| 10 | SEQ | MALPVTALLLPLALLLHAARPDIVMTQAAPSVPVTPGESVSISCRSSKT |
| | ID | LLHSNGNTYLYWFLQRPGQSPQVLIYRMSNLASGVPNRFSGSGSETTF |
| | NO: | TLRISRVEAEDVGIYYCMQHLEYPYTFGGGTKLEIERGGGGSGGGGSG |
| | 56 | GGGSGGGGSEVQLEESGAELVRPGTSVKLSCKASGYTFTSYWMHWIK |
| | | QRPEQGLEWIGRIDPYDSETHYNEKFKDKAILSVDKSSSTAYIQLSSLTS |
| | | DDSAVYYCSRRDAKYDGYALDYWGQGTSVTVSSTPAPRPPTPAPTIAS |
| | | QPLSLRPEACRPAAGGAVHTRGLDFACDFWVLVVVGGVLACYSLLVT |
| | | VAFIIFWVHCHRLPGSYDSTSSDSLYPRGIQFKRPHTVAPWPPAYPPVT |
| | | SYPPLSQPDLLPIPRSPQPLGGSHRTPSSRRDSDGANSVASYENEGASGI |
| | | RGAQAGWGVWGPSWTRLTPVSLPPEPACEDADEDEDDYHNPGYLVV |
| | | LPDSTPATSTAAPSAPALSTPGIRDSAFSMESIDDYVNVPESGESAEASL |
| | | DGSREYVNVSQELHPGAAKTEPAALSSQEAEEVEEEGAPDYENLQELN |
| | | RSKRSRLLHSDYMNMTPRRPGPTRKHYQPYAPPRDFAAYRSRVKFSRS |
| | | ADAPAYKQGQNQLYNELNLGRREEYDVLDKRRGRDPEMGGKPRRKN |
| | | PQEGLYNELQKDKMAEAYSEIGMKGERRRGKGHDGLYQGLSTATKD |
| | | TYDALHMQALPPR |
| 11 | SEQ | MALPVTALLLPLALLLHAARPDIVMTQAAPSVPVTPGESVSISCRSSKT |
| | ID | LLHSNGNTYLYWFLQRPGQSPQVLIYRMSNLASGVPNRFSGSGSETTF |
| | NO: | TLRISRVEAEDVGIYYCMQHLEYPYTFGGGTKLEIERGGGGSGGGGSG |
| | 57 | GGGSGGGGSEVQLEESGAELVRPGTSVKLSCKASGYTFTSYWMHWIK |
| | | QRPEQGLEWIGRIDPYDSETHYNEKFKDKAILSVDKSSSTAYIQLSSLTS |
| | | DDSAVYYCSRRDAKYDGYALDYWGQGTSVTVSSTPAPRPPTPAPTIAS |
| | | QPLSLRPEACRPAAGGAVHTRGLDFACDFWVLVVVGGVLACYSLLVT |
| | | VAFIIFWVRSKRSRLLHSDYMNMTPRRPGPTRKHYQPYAPPRDFAAYR |
| | | SRVKFSRSADAPAYKQGQNQLYNELNLGRREEYDVLDKRRGRDPEM |
| | | GGKPRRKNPQEGLYNELQKDKMAEAYSEIGMKGERRRGKGHDGLYQ |
| | | GLSTATKDTYDALHMQALPPRHCHRLPGSYDSTSSDSLYPRGIQFKRP |
| | | HTVAPWPPAYPPVTSYPPLSQPDLLPIPRSPQPLGGSHRTPSSRRDSDGA |
| | | NSVASYENEGASGIRGAQAGWGVWGPSWTRLTPVSLPPEPACEDADE |
| | | DEDDYHNPGYLVVLPDSTPATSTAAPSAPALSTPGIRDSAFSMESIDDY |
| | | VNVPESGESAEASLDGSREYVNVSQELHPGAAKTEPAALSSQEAEEVE |
| | | EEGAPDYENLQELN |
| 12 | SEQ | MALPVTALLLPLALLLHAARPDIVMTQAAPSVPVTPGESVSISCRSSKT |
| | ID | LLHSNGNTYLYWFLQRPGQSPQVLIYRMSNLASGVPNRFSGSGSETTF |
| | NO: | TLRISRVEAEDVGIYYCMQHLEYPYTFGGGTKLEIERGGGGSGGGGSG |
| | 58 | GGGSGGGGSEVQLEESGAELVRPGTSVKLSCKASGYTFTSYWMHWIK |
| | | QRPEQGLEWIGRIDPYDSETHYNEKFKDKAILSVDKSSSTAYIQLSSLTS |
| | | DDSAVYYCSRRDAKYDGYALDYWGQGTSVTVSSTPAPRPPTPAPTIAS |
| | | QPLSLRPEACRPAAGGAVHTRGLDFACDFWVLVVVGGVLACYSLLVT |
| | | VAFIIFWVKRGRKKLLYIFKQPFMRPVQTTQEEDGCSCRFPEEEEGGCE |
| | | LRVKFSRSADAPAYKQGQNQLYNELNLGRREEYDVLDKRRGRDPEM |
| | | GGKPRRKNPQEGLYNELQKDKMAEAYSEIGMKGERRRGKGHDGLYQ |
| | | GLSTATKDTYDALHMQALPPR |
| 13 | SEQ | MALPVTALLLPLALLLHAARPDIVMTQAAPSVPVTPGESVSISCR |
| | ID | SSKTLLHSNGNTYLYWFLQRPGQSPQVLIYRMSNLASGVPNRFS |
| | NO: | GSGSETTFTLRISRVEAEDVGIYYCMQHLEYPYTFGGGTKLEIER |
| | 59 | GGGGSGGGGSGGGGSGGGGSEVQLEESGAELVRPGTSVKLSCK |
| | | ASGYTFTSYWMHWIKQRPEQGLEWIGRIDPYDSETHYNEKFKD |
| | | KAILSVDKSSSTAYIQLSSLTSDDSAVYYCSRRDAKYDGYALDY |
| | | WGQGTSVTVSSPRASTTTPAPRPPTPAPTIASQPLSLRPEACRPAA |
| | | GGAVHTRGLDFACDFWVLVVVGGVLACYSLLVTVAFIIFWVKR |
| | | GRKKLLYIFKQPFMRPVQTTQEEDGCSCRFPEEEEGGCELKRKK |
| | | QRSRRNDEELETRAHRVATEERGRKPHQIPASTPQNPATSQHPPP |
| | | PPGHRSQAPSHRPPPPGHRVQHQPQKRPPAPSGTQVHQQKGPPL |
| | | PRPRVQPKPPHGAAENSLSPSSNRVKFSRSADAPAYKQGQNQLY |
| | | NELNLGRREEYDVLDKRRGRDPEMGGKPRRKNPQEGLYNELQ |
| | | KDKMAEAYSEIGMKGERRRGKGHDGLYQGLSTATKDTYDALH |
| | | MQALPPR |
| 14 | SEQ | MALPVTALLLPLALLLHAARPDIKMTQSPSSMYVSLGERVTITCKASQ |
| | ID | DINSFLSWFQQKPGKSPKTLIYRANRLVDGVPSRFSGSGSGQDYSLTISS |
| | NO: | LEYEDMEIYYCLQYDEFPYTFGGGTKLEMKRGGGGSEVKLEESGAEL |
| | 60 | VKPGASVKLSCRTSGFNIKDTYIHWVKQRPEQGLKWIGRIDPANGNTK |
| | | YDPKFQDKATVTADTSSNTAYLQLSSLTSEDTAVYYCVTYAYDGNWY |
| | | FDVWGAGTAVTVSSTPAPRPPTPAPTIASQPLSLRPEACRPAAGGAVHT |
| | | RGLDFACDFWVLVVVGGVLACYSLLVTVAFIIFWVRSKRSRLLHSDY |
| | | MNMTPRRPGPTRKHYQPYAPPRDFAAYRSRVKFSRSADAPAYKQGQN |
| | | QLYNELNLGRREEYDVLDKRRGRDPEMGGKPRRKNPQEGLYNELQK |
| | | DKMAEAYSEIGMKGERRRGKGHDGLYQGLSTATKDTYDALHMQALP |
| | | PR |
| 15 | SEQ | MALPVTALLLPLALLLHAARPDIKMTQSPSSMYVSLGERVTITCKASQ |
| | ID | DINSFLSWFQQKPGKSPKTLIYRANRLVDGVPSRFSGSGSGQDYSLTISS |
| | NO: | LEYEDMEIYYCLQYDEFPYTFGGGTKLEMKRGGGGSEVKLEESGAEL |
| | 61 | VKPGASVKLSCRTSGFNIKDTYIHWVKQRPEQGLKWIGRIDPANGNTK |
| | | YDPKFQDKATVTADTSSNTAYLQLSSLTSEDTAVYYCVTYAYDGNWY |
| | | FDVWGAGTAVTVSSTPAPRPPTPAPTIASQPLSLRPEACRPAAGGAVHT |
| | | RGLDFACDFWVLVVVGGVLACYSLLVTVAFIIFWVKRGRKKLLYIFK |
| | | QPFMRPVQTTQEEDGCSCRFPEEEEGGCELRVKFSRSADAPAYKQGQN |
| | | QLYNELNLGRREEYDVLDKRRGRDPEMGGKPRRKNPQEGLYNELQK |
| | | DKMAEAYSEIGMKGERRRGKGHDGLYQGLSTATKDTYDALHMQALP |
| | | PR |
| 16 | SEQ | MALPVTALLLPLALLLHAARPDIKMTQSPSSMYVSLGERVTITCKASQ |
| | ID | DINSFLSWFQQKPGKSPKTLIYRANRLVDGVPSRFSGSGSGQDYSLTISS |
| | NO: | LEYEDMEIYYCLQYDEFPYTFGGGTKLEMKRGGGGSGGGGSEVKLEE |
| | 62 | SGAELVKPGASVKLSCRTSGFNIKDTYIHWVKQRPEQGLKWIGRIDPA |
| | | NGNTKYDPKFQDKATVTADTSSNTAYLQLSSLTSEDTAVYYCVTYAY |
| | | DGNWYFDVWGAGTAVTVSSTPAPRPPTPAPTIASQPLSLRPEACRPAA |
| | | GGAVHTRGLDFACDFWVLVVVGGVLACYSLLVTVAFIIFWVRSKRSR |
| | | LLHSDYMNMTPRRPGPTRKHYQPYAPPRDFAAYRSRVKFSRSADAPA |
| | | YKQGQNQLYNELNLGRREEYDVLDKRRGRDPEMGGKPRRKNPQEGL |
| | | YNELQKDKMAEAYSEIGMKGERRRGKGHDGLYQGLSTATKDTYDAL |
| | | HMQALPPR |
| 17 | SEQ | MALPVTALLLPLALLLHAARPDIKMTQSPSSMYVSLGERVTITCKASQ |
| | ID | DINSFLSWFQQKPGKSPKTLIYRANRLVDGVPSRFSGSGSGQDYSLTISS |
| | NO: | LEYEDMEIYYCLQYDEFPYTFGGGTKLEMKRGGGGSGGGGSEVKLEE |
| | 63 | SGAELVKPGASVKLSCRTSGFNIKDTYIHWVKQRPEQGLKWIGRIDPA |
| | | NGNTKYDPKFQDKATVTADTSSNTAYLQLSSLTSEDTAVYYCVTYAY |
| | | DGNWYFDVWGAGTAVTVSSTPAPRPPTPAPTIASQPLSLRPEACRPAA |
| | | GGAVHTRGLDFACDFWVLVVVGGVLACYSLLVTVAFIIFWVKRGRKK |
| | | LLYIFKQPFMRPVQTTQEEDGCSCRFPEEEEGGCELRVKFSRSADAPAY |
| | | KQGQNQLYNELNLGRREEYDVLDKRRGRDPEMGGKPRRKNPQEGLY |
| | | NELQKDKMAEAYSEIGMKGERRRGKGHDGLYQGLSTATKDTYDALH |
| | | MQALPPR |
| 18 | SEQ | MALPVTALLLPLALLLHAARPDIKMTQSPSSMYVSLGERVTITCKASQ |
| | ID | DINSFLSWFQQKPGKSPKTLIYRANRLVDGVPSRFSGSGSGQDYSLTISS |
| | NO: | LEYEDMEIYYCLQYDEFPYTFGGGTKLEMKRGGGGSGGGGSGGGGSE |
| | 64 | VKLEESGAELVKPGASVKLSCRTSGFNIKDTYIHWVKQRPEQGLKWIG |
| | | RIDPANGNTKYDPKFQDKATVTADTSSNTAYLQLSSLTSEDTAVYYCV |
| | | TYAYDGNWYFDVWGAGTAVTVSSTPAPRPPTPAPTIASQPLSLRPEAC |
| | | RPAAGGAVHTRGLDFACDFWVLVVVGGVLACYSLLVTVAFIIFWVRS |
| | | KRSRLLHSDYMNMTPRRPGPTRKHYQPYAPPRDFAAYRSRVKFSRSA |
| | | DAPAYKQGQNQLYNELNLGRREEYDVLDKRRGRDPEMGGKPRRKNP |
| | | QEGLYNELQKDKMAEAYSEIGMKGERRRGKGHDGLYQGLSTATKDT |
| | | YDALHMQALPPR |
| 19 | SEQ | MALPVTALLLPLALLLHAARPDIKMTQSPSSMYVSLGERVTITCKASQ |
| | ID | DINSFLSWFQQKPGKSPKTLIYRANRLVDGVPSRFSGSGSGQDYSLTISS |
| | NO: | LEYEDMEIYYCLQYDEFPYTFGGGTKLEMKRGGGGSGGGGSGGGGSE |
| | 65 | VKLEESGAELVKPGASVKLSCRTSGFNIKDTYIHWVKQRPEQGLKWIG |
| | | RIDPANGNTKYDPKFQDKATVTADTSSNTAYLQLSSLTSEDTAVYYCV |
| | | TYAYDGNWYFDVWGAGTAVTVSSTPAPRPPTPAPTIASQPLSLRPEAC |
| | | RPAAGGAVHTRGLDFACDFWVLVVVGGVLACYSLLVTVAFIIFWVKR |
| | | GRKKLLYIFKQPFMRPVQTTQEEDGCSCRFPEEEEGGCELRVKFSRSAD |
| | | APAYKQGQNQLYNELNLGRREEYDVLDKRRGRDPEMGGKPRRKNPQ |
| | | EGLYNELQKDKMAEAYSEIGMKGERRRGKGHDGLYQGLSTATKDTY |
| | | DALHMQALPPR |
| 20 | SEQ | MALPVTALLLPLALLLHAARPDIKMTQSPSSMYVSLGERVTITCKASQ |
| | ID | DINSFLSWFQQKPGKSPKTLIYRANRLVDGVPSRFSGSGSGQDYSLTISS |
| | NO: | LEYEDMEIYYCLQYDEFPYTFGGGTKLEMKRGGGGSGGGGSGGGGSG |
| | 66 | GGGSEVKLEESGAELVKPGASVKLSCRTSGFNIKDTYIHWVKQRPEQG |
| | | LKWIGRIDPANGNTKYDPKFQDKATVTADTSSNTAYLQLSSLTSEDTA |
| | | VYYCVTYAYDGNWYFDVWGAGTAVTVSSTPAPRPPTPAPTIASQPLSL |
| | | RPEACRPAAGGAVHTRGLDFACDFWVLVVVGGVLACYSLLVTVAFIIF |
| | | WVRSKRSRLLHSDYMNMTPRRPGPTRKHYQPYAPPRDFAAYRSRVKF |
| | | SRSADAPAYKQGQNQLYNELNLGRREEYDVLDKRRGRDPEMGGKPR |
| | | RKNPQEGLYNELQKDKMAEAYSEIGMKGERRRGKGHDGLYQGLSTA |
| | | TKDTYDALHMQALPPR |
| 21 | SEQ | MALPVTALLLPLALLLHAARPDIKMTQSPSSMYVSLGERVTITCKASQ |
| | ID | DINSFLSWFQQKPGKSPKTLIYRANRLVDGVPSRFSGSGSGQDYSLTISS |
| | NO: | LEYEDMEIYYCLQYDEFPYTFGGGTKLEMKRGGGGSGGGGSGGGGSG |
| | 67 | GGGSEVKLEESGAELVKPGASVKLSCRTSGFNIKDTYIHWVKQRPEQG |
| | | LKWIGRIDPANGNTKYDPKFQDKATVTADTSSNTAYLQLSSLTSEDTA |
| | | VYYCVTYAYDGNWYFDVWGAGTAVTVSSTPAPRPPTPAPTIASQPLSL |
| | | RPEACRPAAGGAVHTRGLDFACDFWVLVVVGGVLACYSLLVTVAFIIF |
| | | WVKRGRKKLLYIFKQPFMRPVQTTQEEDGCSCRFPEEEEGGCELRVKF |
| | | SRSADAPAYKQGQNQLYNELNLGRREEYDVLDKRRGRDPEMGGKPR |
| | | RKNPQEGLYNELQKDKMAEAYSEIGMKGERRRGKGHDGLYQGLSTA |
| | | TKDTYDALHMQALPPR |
| 22 | SEQ | MALPVTALLLPLALLLHAARPDIKMTQSPSSMYVSLGERVTITCKASQ |
| | ID | DINSFLSWFQQKPGKSPKTLIYRANRLVDGVPSRFSGSGSGQDYSLTISS |
| | NO: | LEYEDMEIYYCLQYDEFPYTFGGGTKLEMKRGGGGSGGGGSGGGGSG |
| | 68 | GGGSEVKLEESGAELVKPGASVKLSCRTSGFNIKDTYIHWVKQRPEQG |
| | | LKWIGRIDPANGNTKYDPKFQDKATVTADTSSNTAYLQLSSLTSEDTA |
| | | VYYCVTYAYDGNWYFDVWGAGTAVTVSSTPAPRPPTPAPTIASQPLSL |
| | | RPEACRPAAGGAVHTRGLDFACDFWVLVVVGGVLACYSLLVTVAFIIF |
| | | WVRSKRSRLLHSDYMNMTPRRPGPTRKHYQPYAPPRDFAAYRSKRKK |
| | | QRSRRNDEELETRAHRVATEERGRKPHQIPASTPQNPATSQHPPPPPGH |
| | | RSQAPSHRPPPPGHRVQHQPQKRPPAPSGTQVHQQKGPPLPRPRVQPK |
| | | PPHGAAENSLSPSSNRVKFSRSADAPAYKQGQNQLYNELNLGRREEYD |
| | | VLDKRRGRDPEMGGKPRRKNPQEGLYNELQKDKMAEAYSEIGMKGE |
| | | RRRGKGHDGLYQGLSTATKDTYDALHMQALPPR |

TABLE 4
Sequence of CAR containing CD132 C′ to CD3 zeta

| CAR | SEQ ID | |
|---|---|---|
| Ex. | NO. | Amino Acid Sequence |
| 23 | SEQ ID | MALPVTALLLPLALLLHAARPDIVMTQAAPSVPVTPGESVSIS |
| | NO: 70 | CRSSKTLLHSNGNTYLYWFLQRPGQSPQVLIYRMSNLASGVP |
| | | NRFSGSGSETTFTLRISRVEAEDVGIYYCMQHLEYPYTFGGG |
| | | TKLEIERGGGGSGGGGSGGGGSGGGGSEVQLEESGAELVRP |
| | | GTSVKLSCKASGYTFTSYWMHWIKQRPEQGLEWIGRIDPYD |
| | | SETHYNEKFKDKAILSVDKSSSTAYIQLSSLTSDDSAVYYCSR |
| | | RDAKYDGYALDYWGQGTSVTVSSPRASTTTPAPRPPTPAPTI |
| | | ASQPLSLRPEACRPAAGGAVHTRGLDFACDFWVLVVVGGV |
| | | LACYSLLVTVAFIIFWVRSKRSRLLHSDYMNMTPRRPGPTRK |
| | | HYQPYAPPRDFAAYRSRVKFSRSADAPAYKQGQNQLYNELN |
| | | LGRREEYDVLDKRRGRDPEMGGKPRRKNPQEGLYNELQKD |
| | | KMAEAYSEIGMKGERRRGKGHDGLYQGLSTATKDTYDALH |
| | | MQALPPRFWLERTMPRIPTLKNLEDLVTEYHGNFSAWSGVS |
| | | KGLAESLQPDYSERLCLVSEIPPKGGALGEGPGASPCNQHSP |
| | | YWAPPCYTLKPET |

Similar CAR variations are possible as well. For example CARs may have the form:

|-[(leader)(V_{H})(linker)(V_{L})(hinge)(TMD)(costim)ₙ(effector)]-|,

wherein the sequences are disclosed above in Tables 1 and 2. Accordingly, also provided are CAR Examples 1 a-22a which are identical to those above, except that the positions in the sequence of the V_{H} and V_{L} are reversed.

In a fourth aspect, provided herein are immune effector cells bearing one or more CARs according to the first, second, and/or third aspect. In some embodiments, the CAR-bearing immune effector cell is a chimeric antigen receptor T-cell (CAR-T cell) or a chimeric antigen receptor natural killer cell (CAR-NK cell).

In some embodiments, the CAR-bearing immune effector cell has additional features that reduce or eliminate fratricide, alloreactivity and/or graft-versus-host reactions. In some embodiments, the CAR-bearing immune effector cell further comprises a suicide gene. In some embodiments, CAR-bearing immune effector cell is deficient in at least one or more antigens to which the one or more CARs specifically binds. In some embodiments where the CAR-beating immune effector cell is a CAR-T cell, T cell receptor mediated signaling is blocked in the CAR-T cell. In some embodiments, the CAR-T cell is deficient in a subunit of the T cell receptor complex. In some embodiments, the subunit of the T cell receptor complex is selected from one or more of TCRα, TCRβ, TCRδ, TCRγ, CD3ε, CD3γ, CD34δ, and an extracellular domain of CD3ζ.

Some of these features of CAR-bearing immune effector cells may be introduced through CRISPR/Cas9-mediated editing to delete the gene that encodes the protein for which deficiency is desired. For example, if the CAR-bearing immune effector cell targets CD2, it may be desirable to delete endogenous CD2 and TRAC from the CAR-bearing immune effector cell to reduce or prevent fratricide, The deletion of the CD2 and TRAC genes, in this case, may be confirmed by PCR on the gene-edited cell, sometimes called deep sequencing. Examples of guide RNAs used for gene editing and primers to confirm such editing are shown in Table 4.

TABLE 5
Guide RNAs used in deletion of CD2 and TRAC and PCR primers used to
confirm deletion

| | | | |
|---|---|---|---|
| 1 | Guide RNA | SEQ ID | 2′OMeA(ps)C(ps)A(ps))GCUGACAGGCUCGACACGUU |
| | (CD2) | NO: 71 | UUAGAGCUAGAAAUAGCAAGUUAAAAUAAGGCUA |
| | | | GUCCGUUAUCAACUUGAAAAAGUGGCACCGAGUC |
| | | | GGUGC2′OMe(U(ps)U(ps)U(ps)U |
| 2 | Guide RNA | SEQ ID | 2′OMeG(ps)A(ps)G(ps))AAUCAAAAUCGGUGAAUGUU |
| | (TRAC) | NO: 72 | UUAGAGCUAGAAAUAGCAAGUUAAAAUAAGGCUA |
| | | | GUCCGUUAUCAACUUGAAAAAGUGGCACCGAGUC |
| | | | GGUGC2′OMe(U(ps)U(ps)U(ps)U |
| 3 | PCR Primer | SEQ ID | ACCAAAGATCTCCTGGACTTGT |
| | (CD2) | NO: 73 | |
| | (F, forward) | | |
| 4 | PCR Primer | SEQ ID | GCATGCCTCTGCCTACTGAG |
| | (CD2) | NO: 74 | |
| | (R, reverse) | | |
| 5 | PCR Primer | SEQ ID | GGGGCAAAGAGGGAAATGA |
| | (TRAC) | NO: 75 | |
| | (F, forward) | | |
| 6 | PCR Primer | SEQ ID | GTCAGATTTGTTGCTCCAGGC |
| | (TRAC) | NO: 76 | |
| | (R, reverse) | | |

### Immune Effector Cells

In some embodiments, the CAR-bearing immune effector cell specifically binds at least one cancer-related antigen expressed on a cancer cell. In some embodiments, the cancer cell is a malignant T cell. In some embodiments, the antigen expressed on the malignant T cell is selected from one or more of CD2, CD3, CD4, CD5, CD7, TCRα(TRAC), and TCRβ. In some embodiments, the cancer cell is a malignant plasma cell. In some embodiments, the antigen expressed on the malignant plasma cell is selected from one or more of BCMA, CS1, CD38, CD79A, CD79B, CD138, and CD19. In some embodiments, the cancer cell is a malignant B cell. In some embodiments, the antigen expressed on a malignant B cell is selected from one or more of CD19, CD20, CD21, CD22, CD23, CD24, CD25, CD27, CD38, and CD45. the antigen expressed on a malignant B cell is selected from one or more of CD19 and CD20.

In some embodiments, the CAR-NK cell is selected from a car-bearing ML, MIL, and iNKT cell.

Immune effector cells as disclosed herein may include T cells, NK cells, iNKT cells, and others, for example macrophages, and subtypes thereof.

Any of these immune effector cells may be transduced with a CAR using techniques known in the art. The resulting CAR-bearing immune effector cells may be used in the immunotherapy of disease, for example cancer, by adoptive cell transfer (ACT) into a subject in need. CAR-bearing immune effector cells include CAR-T cells, CAR-NK cells (and subtypes thereof, such as CAR-ML NK cells and CAR-CIMLs), CAR-iNKT cells, and CAR-macrophages.

Immune effector cells for use in ACT may be autologous or allogeneic. In some embodiments, the use of allogeneic cells permits deliberate polymorphic mismatch between donor and recipient, which offers certain advantages discussed below.

### T Cells

T cells are immune cells which express a T cell receptor (TCR) on their surface. Effector T cells include cytotoxic (CD8+) T cells, helper (CD4+) T cells, viral-specific cytotoxic T cells, memory T cells, gamma delta (γδ) T cells.

T cells may be primary T cells, or may be derived from progenitor cells. T cells can be derived from various sources, including peripheral or cord blood cells, stem cells, or induced pluripotent stem cells (iPSCs), Methods of enriching/isolating, differentiating, and otherwise producing T cells are known in the art.

iNKT Cells

Invariant natural killer T cells, also called iNKT cells or type-I NKT cells, represent a distinct lymphocyte population, characterized by expression of an invariant T cell receptor α-chain and certain TCR β-chains (Vα24-Jα18 combined with Vβ11). iNKT TCR-mediated responses are restricted by CD1d, a member of the non-polymorphic CD1 antigen presenting protein family, which promotes the presentation of endogenous and pathogen-derived lipid antigens to the TCR. The prototypical ligand for invariant receptor is α-Galactosylceramide (αGalCer). Upon binding of the invariant TCR to CD1d-αGalCer, iNKT will expand. The CD1d gene is monomorphic and expressed by only a few cell types, limiting the potential toxicity of NKT cells in the autologous or allogeneic settings.

### NK Cells

Natural killer (NK) cells are traditionally considered innate immune effector lymphocytes which mediate host defense against pathogens and antitumor immune responses by targeting and eliminating abnormal or stressed cells not by antigen recognition or prior sensitization, but through the integration of signals from activating and inhibitory receptors. Natural killer (NK) cells are an alternative to T cells for allogeneic cellular immunotherapy since they have been administered safely without major toxicity, do not cause graft versus host disease (GvHD), naturally recognize and eliminate malignant cells, and are amendable to cellular engineering.

NK cells may be primary NK cells, or may be derived from progenitor cells. NK cells can be derived from various sources, including peripheral or cord blood cells, stem cells, or induced pluripotent stem cells (iPSCs), Methods of enriching/isolating, differentiating, and otherwise producing NK cells are known in the art.

NK cells express a number of transmembrane (TM) adapters that signal activation, that are triggered via association with activating receptors. This provides an NK cell specific signal enhancement via engineering the TM domains from activating receptors, and thereby harness endogenous adapters. The TM adapter can be any endogenous TM adapter capable of signaling activation. In some embodiments, the TM adapter may be chosen from FceR1γ (ITAMx1), CD3ζ (ITAMx3), DAP12 (ITAMx1), or DAP10 (YxxM/YINM), NKG2D, FcγRIIIa, NKp44, NKp30, NKp46, actKIR, NKG2C, CD8α, and IL15Rb.

Memory-Like NK cells

In addition to their innate cytotoxic and immunostimulatory activity, NK cells constitute a heterogeneous and versatile cell subset, including persistent memory-like NK populations that mount a robust recall response. ML-NK cells can be produced by stimulation by pro-inflammatory cytokines or activating receptor pathways, either naturally or artificially. ML-NK cells produced by cytokine activation have been used clinically in the setting of leukemia immunotherapy.

Increased CD56, Ki-67, NKG2A, and increased activating receptors NKG2D, NKp30, and NKp44 have been observed in in vivo differentiated ML NK cells. In addition, in vivo differentiation showed modest decreases in the median expression of CD16 and CD11b. Increased frequency of TRAIL, CD69, CD62L, NKG2A, and NKp30-positive NK cells were observed in ML NK cells compared with both ACT and BL NK cells, whereas the frequencies of CD27+ and CD127+ NK cells were reduced. Finally, unlike in vitro differentiated ML NK cells, in vivo differentiated ML NK cells did not express CD25.

### Cytokine-Induced Memory-Like Natural Killer Cells (CIML-NKs)

NK cells may be induced to acquire a memory-like phenotype, for example by preactivation with combinations of cytokines, such as interleukin-12 (IL-12), IL-15, and IL-18. These cytokine-induced memory-like (CIML) NK cells (CIML-NKs or CIMLs) exhibit enhanced response upon restimulation with the cytokines or triggering via activating receptors. CIML NK cells may be produced by activation with cytokines such as IL-12, IL-15, and IL-18 and their related family members, or functional fragments thereof, or fusion proteins comprising functional fragments thereof.

CIML NK cells may be identified by their method of production. CIML cells can be produced by differentiated cytokine-activated (i.e., CIML) NK cells.

CIML NK cells typically exhibit differential cell surface protein expression patterns when compared to traditional NK cells. Such expression patterns are known in the art and may comprise, for example, increased CD56, CD56 subset CD56dim, CD56 subset CD56bright, CD16, CD94, NKG2A, NKG2D, CD62L, CD25, NKp30, NKp44, and NKp46 (compared to control NK cells) in CIML NK cells (see e.g., Romee et al. Sci Transl Med. 2016 Sep. 21; 8(357):357). Memory-like (ML) and cytokine induced memory-like (CIML) NK cells may also be identified by observed in vitro and in vivo properties, such as enhanced effector functions such as cytotoxicity, improved persistence, increased IFN-γ production, and the like.

NK cells can be activated using cytokines, such as IL-12/15/18. The NK cells can be incubated in the presence of the cytokines for an amount of time sufficient to form cytokine-induced memory-like (CIML) NK cells. Such techniques are known in the art.

In a fifth aspect, provided herein is a therapeutic composition comprising a population of CAR-bearing immune effector cells according to the fourth aspect of the invention and at least one therapeutically acceptable diluent, carrier and/or adjuvant.

In a sixth aspect, provided herein is a method for treatment of cancer in a patient comprising administering a population of CAR-bearing immune effector cells according to the third aspect of the invention or a therapeutic composition according to the fourth aspect of the invention to a cancer patient. In some embodiments, the immune effector cell or a population of CAR-bearing immune effector cells is a CAR-T cell or a population of CAR-T cells, according to the third aspect of the invention.

In some embodiments, the cancer is a hematologic malignancy. In some embodiments, the hematologic malignancy is a T-cell malignancy. In some embodiments, the T cell malignancy is T-cell acute lymphoblastic leukemia (T-ALL). In some embodiments, the T cell malignancy is non-Hodgkin's lymphoma. In some embodiments, the T cell malignancy is T-cell chronic lymphocytic leukemia (T-CLL). In some embodiments, the T cell malignancy is selected from T-cell acute lymphoblastic leukemia/lymphoma (T-ALL), human T-cell leukemia virus type 1-positive (HTLV-1 +) adult T-cell leukemia/lymphoma (ATL), T-cell prolymphocytic leukemia (T-PLL), Adult T-cell lymphoma/leukemia (HTLV-1 associated), Aggressive NK-cell leukemia, Anaplastic large-cell lymphoma (ALCL), ALK positive, Anaplastic large-cell lymphoma (ALCL), ALK negative, Angioimmunoblastic T-cell lymphoma (AITL), Breast implant-associated anaplastic large-cell lymphoma, Chronic lymphoproliferative disorder of NK cells, Extra nodal NK/T-cell lymphoma, nasal type, Enteropathy-type T-cell lymphoma, Follicular T-cell lymphoma, Hepatosplenic T-cell lymphoma, Indolent T-cell lymphoproliferative disorder of the GI tract, Monomorphic epitheliotropic intestinal T-cell lymphoma, Mycosis fungoides, Nodal peripheral T-cell lymphoma with TFH phenotype, Peripheral T-cell lymphoma (PTCL), NOS, Primary cutaneous γδ T-cell lymphoma, Primary cutaneous CD8+ aggressive epidermotropic cytotoxic T-cell lymphoma, Primary cutaneous acral CD8+ T-cell lymphoma, Primary cutaneous CD4+ small/medium T-cell lymphoproliferative disorders [Primary cutaneous anaplastic large-cell lymphoma (C-ALCL), lymphoid papulosis], Sezary syndrome, Subcutaneous, panniculitis-like T-cell lymphoma, Systemic EBV+ T-cell lymphoma of childhood, and T-cell large granular lymphocytic leukemia (LGL).

In some embodiments, the hematologic malignancy is a plasma cell malignancy. In some embodiments, the plasma cell malignancy is selected from lymphoplasmacytic lymphoma, plasmacytoma and multiple myeloma.

In some embodiments, the hematologic malignancy is a B-cell cell malignancy. In some embodiments, the B-cell malignancy is selected from wherein the B-cell malignancy is selected from diffuse large B cell lymphoma (DLBCL), chronic lymphocytic leukemia (CLL)/small lymphocytic lymphoma (SLL), and B cell-precursor acute lymphoblastic leukemia (ALL).

### Definitions

Unless specifically defined herein, all technical and scientific terms used have the same meaning as commonly understood by a skilled artisan in the fields of gene therapy, biochemistry, genetics, immunology, and molecular biology. All disclosed compositions and methods similar or equivalent to those described herein can be used in the practice or testing of the present disclosure.

“Antigen binding domain” means an oligo- or polypeptide that is capable of binding a ligand. Generally, the antigen binding domain will be capable of interacting with a cell surface molecule which may be an antigen, a receptor, a peptide ligand, a protein ligand of the target, or a polypeptide of the target. The extracellular ligand-binding domain can specifically bind to an antigen with an affinity constant or affinity of interaction (K_{D}) from about 0.1 pM to about 100 nM, for example from about 0.1 pM to about 10 pM, or from about 0.1 pM to about 1 pM. Methods for determining the affinity constant or affinity of interaction (K_{D}) are well-known in the art. In some instances, the extracellular ligand-binding domain is chosen to recognize a ligand that acts as a cell surface marker on target cells associated with particular disease states. In some embodiments, the antigen binding domain comprises a single chain antibody fragment (scFv) comprising a V_{H} chain, a peptide linker and a V_{L} chain. In some embodiments, the peptide linker between the V_{H} and V_{L} chains has the amino acid sequence GGGGS₍₁₋₆₎, for example GGGGS₍₁₋₄₎.

“Biologically active fragment” generally means a fragment of a polypeptide that substantially retains the biological activity of the intact polypeptide in the context of CAR function.

A “CAR-bearing immune effector cell” is an immune effector cell which has been transduced with at least one CAR. A “CAR-T cell” is a T cell which has been transduced with at least one CAR; CAR-T cells can be mono, dual, or tandem CAR-T cells. CAR-T cells can be autologous, meaning that they are engineered from a subject's own cells, or allogeneic, meaning that the cells are sourced from a healthy donor, and in many cases, engineered so as not to provoke a host-vs-graft or graft-vs-host reaction. Donor cells may also be sourced from cord blood or generated from induced pluripotent stem cells.

“CAR-T cell” means a T cell which expresses a chimeric antigen receptor. The T cell expressing a CAR molecule may be a helper T cell, a cytotoxic T cell, a viral-specific cytotoxic T cell, a memory T cell, or a gamma delta (γδ) T cell.

“CD4 domain” means the CD4 polypeptide or a biologically active fragment thereof that retains the function of recruiting Lck to the inner cell membrane of an immune effector cell. It can also include a synthetic amino acid sequence having at least 95%, at least 98%, or at least 99% sequence identity thereto.

“CD8a leader”, “CD8α leader”, “CD8a leader polypeptide” or “CD8α leader polypeptide” means the CD8a protein or a biologically fragment thereof that functions to initiate translocation of the extracellular domain of the CAR through the immune effector cell membrane to its membrane surface. A biologically active fragment in this case is defined as a fragment of at least. 10 amino acids of the CD8α signal peptide that directs the appended polypeptide to the cell membrane and/or cell surface. Examples of functional fragments of the human CD8α signal peptide are given in Table 1.

“Chimeric antigen receptor (CAR), means a recombinant fusion protein comprising: 1) an extracellular ligand-binding domain, also referred to herein as an antigen binding domain an antigen-domain, 2) a transmembrane domain, and 3) a signaling transducing domain.

“Co-stimulatory domain” means an oligopeptide or a polypeptide that enhances the signaling capability of a signaling domain.

“Deficient in at least one or more antigens to which the one or more CARs specifically binds” means the corollary antigen of the immune effector cell to which the antigen binding domain binds is modified such that the chimeric antigen receptor no longer specifically binds the modified antigen. Stated differently, a cell that “is deficient in” an antigen is one that does not express, expresses a reduced amount of, or expresses an altered type of, the antigen, such that chimeric antigen receptors or other proteins which would typically bind the antigen have a reduced or eliminated ability to do so.

A “dual” CAR-bearing immune effector cell, for example a “dual CAR-T cell” or a “dual CAR-NK cell” means an engineered immune effector cell (e.g., T or NK cell) with two distinct chimeric antigen receptor polypeptides with affinity to different target antigen expressed within the same effector cell, wherein each CAR functions independently. The CAR may be expressed from single or multiple polynucleotide sequences.

“Fratricide” means a process which occurs when a CAR-bearing immune effector cell becomes the target of, and is killed by, another CAR-bearing immune effector cell comprising the same chimeric antigen receptor as the target of the CAR-bearing immune effector cell, because the targeted cell expresses the antigen specifically recognized by the chimeric antigen receptor on both cells.

As used herein, the phrase “from a proximal N-terminus to a distal C-terminus,” when used to describe elements comprised in a chimeric antigen receptor, does not exclude variations known to those of skill in the art and linking sequences between the elements.

“Hinge” means any oligo- or polypeptide that functions to link the transmembrane domain to the antigen binding domain. In particular, the hinge is used to provide more flexibility and accessibility for the antigen binding domain

“Immediately distal to” means in proximity on the C-terminal side of an element, such as a linker or biologically functional domain. It can mean contiguous with, or in such close proximity as to not interfere with its own function, of the function of the element, or their combined function.

“Immediately proximal to” means in proximity on the N-terminal side of an element, such as a linker or biologically functional domain. It can mean contiguous with, or in such close proximity as to not interfere with its own function, of the function of the element, or their combined function.

“Immune effector cell” means a cell derived from a cell that provides a function within the immune system and includes, without limitation, T cells, natural killer (NK) cells, monocytes, dendritic cells, B cells and macrophages.

As used herein, “LAT” (sometimes called Lat, LAT1, pp36, or IMD52) means linker of activated T cells. “LAT domain” means the LAT polypeptide or a biologically active fragment thereof that retains the function of recruiting Lck to the inner cell membrane of an immune effector cell. It can also include a synthetic amino acid sequence having at least 95%, at least 98%, or at least 99% sequence identity thereto.

As used herein, “Lck” (sometimes called LCK proto-oncogene, Src family tyrosine kinase, IMD22, LSK, YT16, p561ck, or pp581ck) means lymphocyte-specific protein tyrosine kinase.

“Signaling domain” or “signal transducing domain” means a signal transducing domain or intracellular signaling domain of a CAR which is responsible for intracellular signaling following the binding of the antigen binding domain to the target resulting in the activation of the immune cell and immune response. In other words, the signal transducing domain is responsible for the activation of at least one of the normal effector functions of the immune cell in which the CAR is expressed.

“Suicide gene” is a gene that causes an immune effector cell to undergo cell death when the cell exceeds a certain level of immune effector activity. Suitable suicide gene systems known in the art include, but are not limited to, several herpes simplex virus thymidine kinase (HSVtk)/ganciclovir (GCV) or inducible caspase 9 proteins. In one embodiment, the suicide gene is a chimeric CD34/thymidine kinase.

A “tandem” CAR-bearing immune effector cell, for example a “tandem CAR-T cell” or a “tandem CAR-NK cell” means an engineered immune effector cell (e.g., T or NK cell) with a single chimeric antigen polypeptide comprising two or more distinct extracellular ligand-binding domains capable of interacting with two or more different cell surface molecules, wherein extracellular ligand-binding domains are linked together by a flexible linker and share one or more effector/costimulatory domains, wherein the binding of the first or the second extracellular ligand-binding domain will signal through one or more the costimulatory domains and a signaling transducing domain. A tandem CAR may be bispecific or trispecific.

“Therapeutic composition” refers to substances which are suitable for use in contact with the tissues of patients without undue toxicity, irritation, and allergic response, are commensurate with a reasonable benefit/risk ratio, and/or are effective for their intended use.

“Transmembrane domain” means a generally hydrophobic peptide or polypeptide that traverses the cell membrane, anchors the CAR to the T cell surface, and connects the antigen-binding domain to the signaling transducing domain, impacting the expression of the CAR on the T cell surface. The distinguishing feature of the transmembrane domain in the present disclosure is the ability to be expressed at the surface of an immune cell to direct an immune cell response against a pre-defined target cell.

“Truncated CD34 domain” or “TrCD34 domain” means a fragment of CD34 polypeptide.

### EXAMPLES

The following examples are intended to further illustrate certain non-limiting embodiments of the invention and are not intended to limit in any way the scope of the invention.

### Example 1—Method of Making CARs

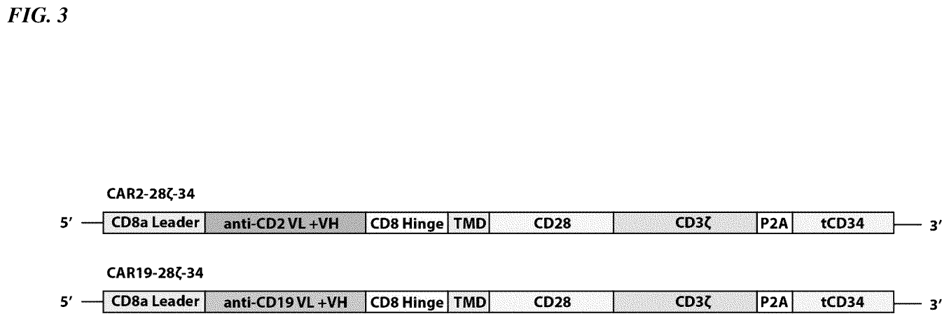

The heavy and light chain sequences were obtained from Hybridoma 35.1 ATCC HB-222 via commercial sequencing (Genescript). To generate the CD2-28ζ construct, the scFv DNA sequence was synthesized and cloned into the backbone of a 2^{nd} generation CAR with a CD28 internal co-stimulatory domain in the pLVM lentiviral vector. The CAR construct was modified to express hCD34 via a P2A peptide. Expression of CD34 allowed for detection of the CAR following viral transduction as well as purification of CAR-T positive cells using anti-hCD34 positive magnetic beads. A 2^{nd} generation CD19 targeting construct, CD19-28ζ, was generated for use as a negative, non-targeting control. A schematic of the constructs is shown in FIG. 3. Additional CARs were and may be constructed according to these methods using variations known in the art.

### Example 2—Viral Vector Production

Lentivirus was produced using the Lenti-X 293T cell line (Takara Bio, Mountain View, CA). The Lenti-X 293T cells were transfected with the CAR lentiviral vector and packaging plasmids, pMD.Lg/rRRE, pMD.G, and pRSV.REV¹⁴, using Lipofectamine 2000 Transfection Reagent from Invitrogen according to the manufacturer's instructions. 36 hours following transfection the viral supernatant was harvested, filtered to remove cell debris, and concentrated via ultracentrifugation at 4° C. for 90 minutes at 25,000 rpm (Optima LE-80K Ultracentrifuge, Beckman Coulter, Indianapolis IN). Once concentrated, the virus was re-suspended in phosphate buffered saline.

### Example 3—CRISPR/Cas9 Editing to Remove Endogenous CD2 and/or TRAC Genes

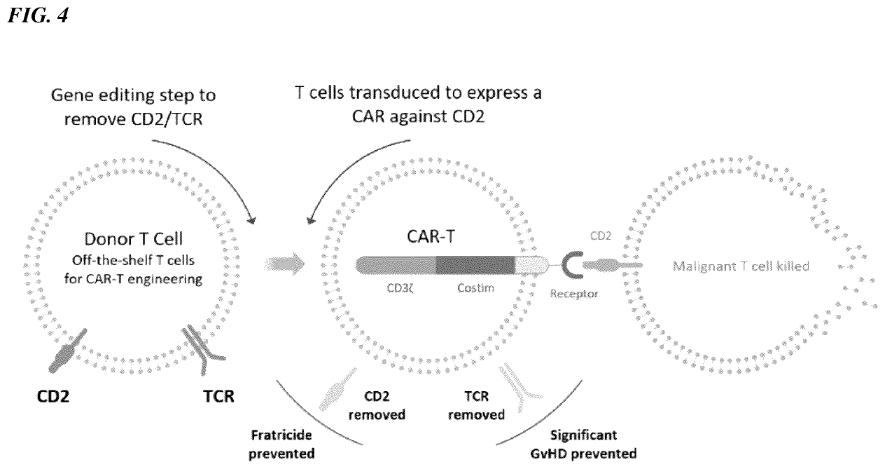

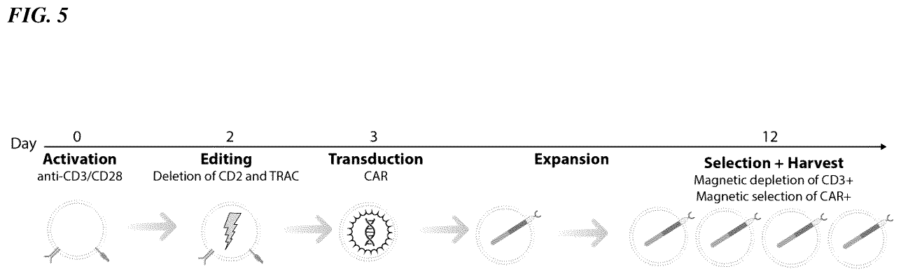

T cells were cultured in Xcyte media supplemented with 50 U/mL IL-2, 10 ng/ml IL-2, 10 ng/ml IL-15, and 10 ng/ml IL7 in the presence of anti-CD3/CD28 beads (Bead to cell ratio 3:1). On day +2 post activation, stimulation was removed and 1×10⁷ T cells were electroporated in 100 μl MaxCyte buffer containing 15 μg spCas9 (Trilink CA.) and 20 μg of each gRNA (IDT) using a MaxCyte GT, program ‘expanded T cell 2’. The cells were transduced with CD2-28ζ or CD19-28ζ lentiviral particles in the presence of polybrene (Sigma Aldrich, St Louis MO) (final concentration 6 μg/ml) on day +3. The cells were then expanded for 6 days following transduction before being used in later experiments. A schematic of CAR-T cell design and testing is shown in FIG. 4. A timeline of CAR-T cell preparation is shown in FIG. 5.

gRNA Sequences

CD2 gRNA:
(SEQ ID NO: 71)
5′_2′OMe(A(ps)C(ps)A(ps))GCUGACAGGCUCGACACGUUUUAG
AGCUAGAAAUAGCAAGUUAAAAUAAGGCUAGUCCGUUAUCAACUUGAAA
AAGUGGCACCGAGUCGGUGC2′OMe(U(ps)U(ps)U(ps)U_3′
TRAC gRNA:
(SEQ ID NO: 72)
5′_2′OMe(G(ps)A(ps)G(ps))AAUCAAAAUCGGUGAAUGUUUUAG
AGCUAGAAAUAGCAAGUUAAAAUAAGGCUAGUCCGUUAUCAACUUGAAA
AAGUGGCACCGAGUCGGUGC2′OMe(U(ps)U(ps)U(ps)U_3′
RNA; (ps) indicate phosphorothioate. Underlined bases denote target sequence.

### Example 4—Targeted Deep Sequencing to Confirm Editing

The CD2 locus was amplified with primers F_ACCAAAGATCTCCTGGACTTGT (SEQ ID NO:73) and R_GCATGCCTCTGCCTACTGAG (SEQ ID NO:74).

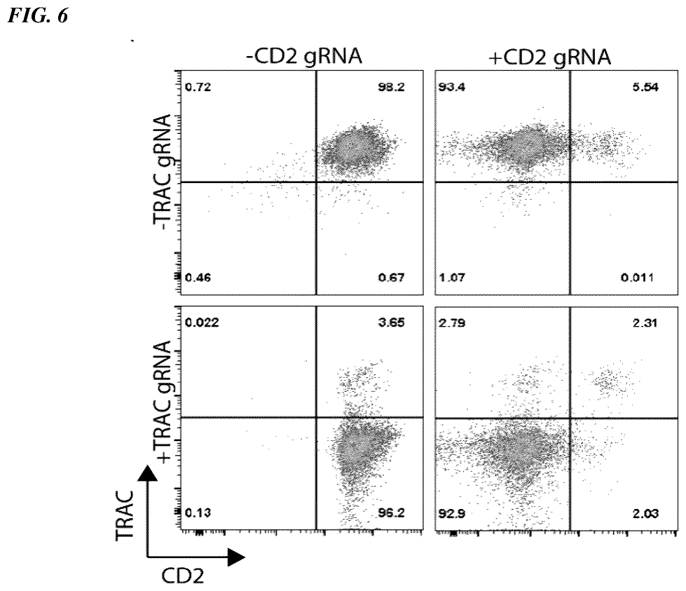

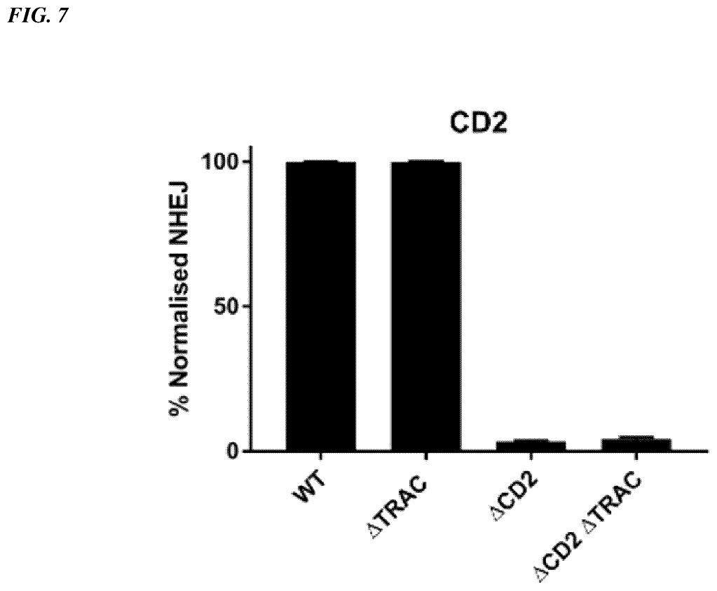

The TRAC Locus was amplified with primers F_GGGGCAAAGAGGGAAATGA (SEQ ID NO:75) and R_GTCAGATTTGTTGCTCCAGGC (SEQ ID NO:76). PCR products were sequenced using the Illumina MiSeq platform (San Diego CA). Editing efficiencies were determined as a percentage of sequencing reads with indels aligned to reads obtained from WT cells. The results are shown in FIG. 6, as shown by FACS, and FIG. 7, converted to bar chart. Removal of both CD2 and TRAC was successful.

### Example 5: CAR-Bearing Immune Effector Cells

CAR-bearing immune effector cells were constructed, optionally with a genome editing step, as described in Example 3 above, to effect deletion or suppression of one or more surface proteins. Such surface proteins included, for example, CD2 and those that form part of the TCR complex (TRAC in Example 3), which may induce GvHD if the cells are administered to patients in the allogeneic setting, or those that are the target antigen of the CAR, which may induce fratricide if expression of the antigen on CAR-T is not suppressed.

For example, in one protocol, on Day 0, CD4+ CD⁺ T cells were thawed in a cell culture media. The required number of cells were centrifuged at 200×g for 10 minutes at room temperature. Supernatant was removed completely, cells resuspended in cell culture media (TexMacs) supplemented with IL-7 (10 ng/ml) and IL-15 (10 ng/ml) at concentration of 1×10⁶/ml. T cells were stimulated with Miltenyi research grade TransAct™ (10 μl/ml).

On day 1, the required amount of viral vector comprising CAR was added to the activated cells at the required M.O.I (Multiplicity of Infection). Cells and virus were mixed and placed back in incubator at 37° C.

TABLE 5

| CAR | SEQ ID | | | | | |
|---|---|---|---|---|---|---|
| Ex. No. | NO: | Media | Stimulation | Cas9 p | gRNA | CAR-T cell |
| | | TexMacs | T Cell | — | — | — |
| | | | TransAct ™ | | | |
| | | | (10 μl/ml) | | | |
| 1 | 47 | TexMacs | T Cell | 10 μg | 20 μg | T1 |
| | | | TransAct ™ | | TRAC | |
| | | | (50 μl) | | | |
| 2 | 48 | TexMacs | T Cell | 10 μg | 20 μg | T2 |
| | | | TransAct ™ | | TRAC | |
| | | | (50 μl) | | | |
| 3 | 49 | TexMacs | T Cell | 10 μg | 20 μg | T3 |
| | | | TransAct ™ | | TRAC | |
| | | | (50 μl) | | | |
| 4 | 50 | TexMacs | T Cell | 10 μg | 20 μg | T4 |
| | | | TransAct ™ | | TRAC | |
| | | | (50 μl) | | | |
| 5 | 51 | TexMacs | T Cell | 10 μg | 20 μg | T5 |
| | | | TransAct ™ | | TRAC | |
| | | | (50 μl) | | | |
| 6 | 52 | TexMacs | T Cell | 10 μg | 20 μg | T6 |
| | | | TransAct ™ | | TRAC | |
| | | | (50 μl) | | | |
| 7 | 53 | TexMacs | T Cell | 10 μg | 20 μg | T7 |
| | | | TransAct ™ | | TRAC | |
| | | | (50 μl) | | | |
| 8 | 54 | TexMacs | T Cell | 10 μg | 20 μg | T8 |
| | | | TransAct ™ | | TRAC | |
| | | | (50 μl) | | | |
| 9 | 55 | TexMacs | T Cell | 10 μg | 20 μg | T9 |
| | | | TransAct ™ | | TRAC | |
| | | | (50 μl) | | | |
| 10 | 56 | TexMacs | T Cell | 10 μg | 20 μg | T10 |
| | | | TransAct ™ | | TRAC | |
| | | | (50 μl) | | | |
| 11 | 57 | TexMacs | T Cell | 10 μg | 20 μg | T11 |
| | | | TransAct ™ | | TRAC | |
| | | | (50 μl) | | | |
| 12 | 58 | TexMacs | T Cell | 10 μg | 20 μg | T12 |
| | | | TransAct ™ | | TRAC | |
| | | | (50 μl) | | | |
| 13 | 59 | TexMacs | T Cell | 10 μg | 20 μg | T13 |
| | | | TransAct ™ | | TRAC | |
| | | | (50 μl) | | | |
| 14 | 60 | TexMacs | T Cell | 10 μg | 20 μg | T14 |
| | | | TransAct ™ | | TRAC | |
| | | | (50 μl) | | | |
| 15 | 61 | TexMacs | T Cell | 10 μg | 20 μg | T15 |
| | | | TransAct ™ | | TRAC | |
| | | | (50 μl) | | | |
| 16 | 62 | TexMacs | T Cell | 10 μg | 20 μg | T16 |
| | | | TransAct ™ | | TRAC | |
| | | | (50 μl) | | | |
| 17 | 63 | TexMacs | T Cell | 10 μg | 20 μg | T17 |
| | | | TransAct ™ | | TRAC | |
| | | | (50 μl) | | | |
| 18 | 64 | TexMacs | T Cell | 10 μg | 20 μg | T18 |
| | | | TransAct ™ | | TRAC | |
| | | | (50 μl) | | | |
| 29 | 65 | TexMacs | T Cell | 10 μg | 20 μg | T19 |
| | | | TransAct ™ | | TRAC | |
| | | | (50 μl) | | | |
| 20 | 66 | TexMacs | T Cell | 10 μg | 20 μg | T20 |
| | | | TransAct ™ | | TRAC | |
| | | | (50 μl) | | | |
| 21 | 67 | TexMacs | T Cell | 10 μg | 20 μg | T21 |
| | | | TransAct ™ | | TRAC | |
| | | | (50 μl) | | | |
| 22 | 68 | TexMacs | T Cell | 10 μg | 20 μg | T22 |
| | | | TransAct ™ | | TRAC | |
| | | | (50 μl) | | | |
| 23 | 70 | TexMacs | T Cell | 10 μg | 20 μg | T23 |
| | | | TransAct ™ | | TRAC | |
| | | | (50 μl) | | | |
| 24 | — | TexMacs | T Cell | 10 μg | 20 μg | T24 |
| | | | TransAct ™ | | TRAC | |
| | | | (50 μl) | | | |
| 25 | — | TexMacs | T Cell | 10 μg | 20 μg | T25 |
| | | | TransAct ™ | | TRAC | |
| | | | (50 μl) | | | |

On day 3, activated cells were washed to remove stimulation.

Where genome editing was desired, cells were harvested and counted. The required number of cells were centrifuged at 100×g for 10 minutes at room temperature.

Supernatant was removed completely, cells resuspended in Electroporation buffer (1 ml ) (e.g. Maxcyte EP buffer) and transferred to a microcentrifuge tube, and centrifuged at 100×g for minutes at room temperature. Supernatant was removed completely, and cells then resuspended in electroporation buffer (e.g., MaxCyte EP buffer), at the desired concentration (e.g. 5×10⁷/ml).

Commercially available Cas9 Protein (10 μg) and commercially synthesized gRNA (20 μg) were complexed at room temperature for 10 minutes.

Cells (100 μl) were transferred to the tube containing complexed Cas9/gRNA, gently mixed, and everything transferred into a MaxCyte OC100 cuvette. Electroporation was thereafter commenced using Maxcyte program Expanded T cell 2. After this procedure, the activated cells were transferred to 10 ml of pre-warmed media and returned to the incubator to expand for an additional 7-12 days.

FACS analysis was used to show the purity of CAR-transduced cells (CAR expression and target gene deletion).

### Example 6—Cell Lines and Cell Line Preparation

CD2 positive T-ALL cell lines, MOLT-3 (ACC 84), Jurkat (ACC 282), were obtained from DSMZ-German collection of Microorganisms and Cell Cultures (Leibniz, Germany) CD2 positive cutaneous T cell lymphoma cell line, HH (CRL-2105) B cell acute lymphoblastic leukemia cell line, NALM6 ATCC® CRL-3273 were obtained from ATCC Global Bioresource Center (Manassas VA). The cell lines were mycoplasma tested upon receipt. HH and NALM6 cell lines were transduced with EF1α^{CBR-GFP} lentivirus at an MOI of 5:1. Following transduction, the GFP positive cells were sorted and expanded to establish pure CBR-GFP cell lines (HH^{CBR-GFP} and NALM6^{CBR-GFP}).

### Example 7—Killing of T-ALL and CTCL cells by CAR-T Cells: Chromium Release

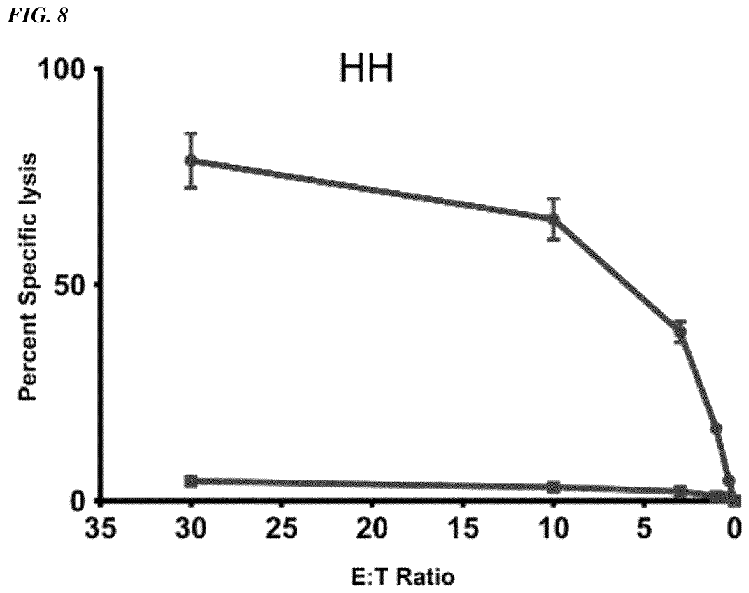

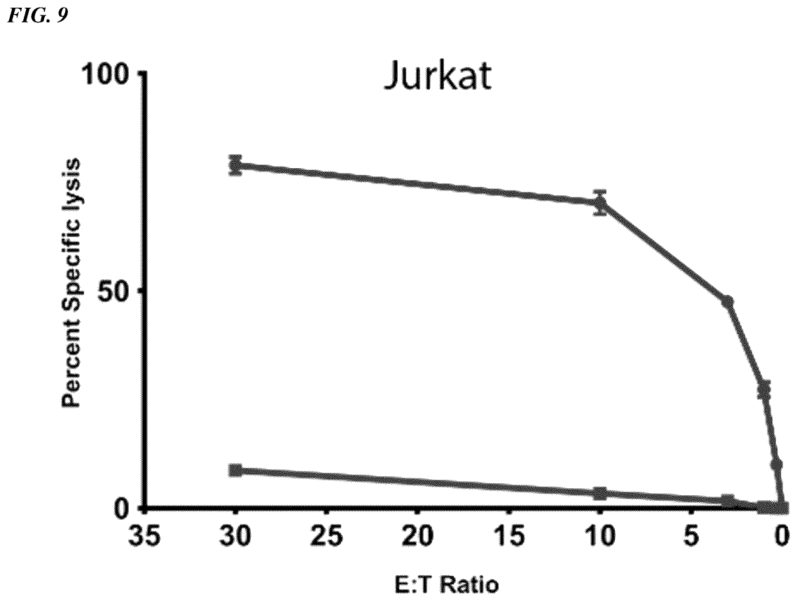

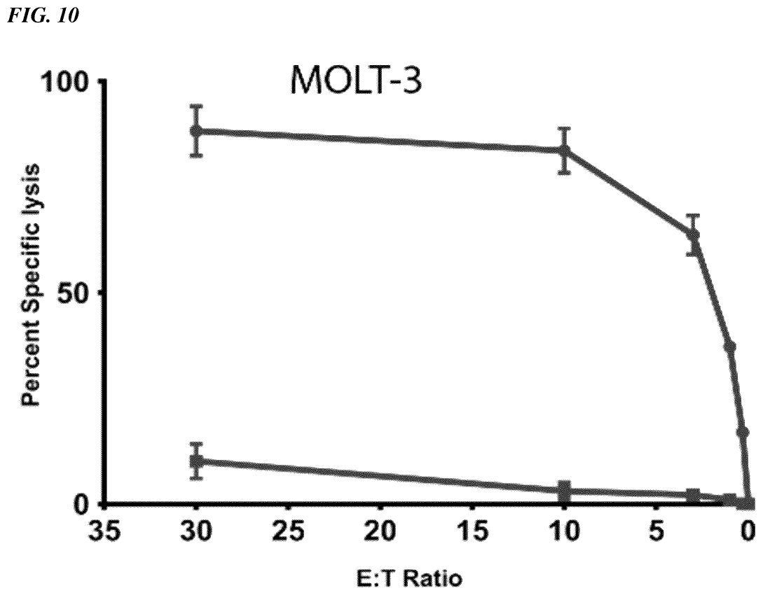

UCART2 or UCART19 cells were incubated with [⁵¹Cr]-labeled Jurkat, MOLT-3 and HH cell lines (1×10 4 total cells/well) at an effector: target [E:T] ratio ranging from 25:1 to 0.25:1 in RPMI supplemented with 5% fetal calf serum. The chromium release assay was performed according to a standard protocol. The results are shown in FIG. 8 (for HH cells), FIG. 9 (for Jurkat cells), and FIG. 10 (for MOLT-3 cells). All 3 cell lines were efficiently killed bu UCART2 cells.

### Example 8—T Cell Phenotype Analysis

Cultured T cells were washed in PBS/0.1% BSA and re-suspended at 1×10⁶ cells in 50 uL Brilliant Buffer (BD Biosciences) supplemented with 4% rat serum for 15 minutes at 4C. Cells were then incubated for 30 minutes at 4° C. in 100 μL of Brilliant Buffer using the following antibody fluorophore conjugates (all from BD Biosciences unless otherwise noted): CD2 BUV395, CD8 BUV496, CD3 BUV661, CD38 BV421, Tim-3 BV450 (BioLegend), PDL1 BV510 (BioLegend), PD1 BV605 (BioLegend), CD4 BV650, CD45 BV711, CD196/CCR6 (BioLegend), CD45RA, Cy5.5, CD183/CXCR3 PE, CD34 PE-Cy7 (BioLegend), Tigit APC (BioLegend), CD127 APC-eF780 (eBioscience), CD62L AF700 (BioLegend). Cells were then washed twice in PBS/0.1% BSA and data acquired on a ZE5 (Yeti) cytometer (BioRad/Propel Labs). Compensation and analyses were performed on FlowJo V10 (TreeStar) using fluorescence minus one (FMO) controls. Statistical analyses were performed on GraphPad Prism 7.

### Example 9—Xenogeneic Mouse Model of CTCL

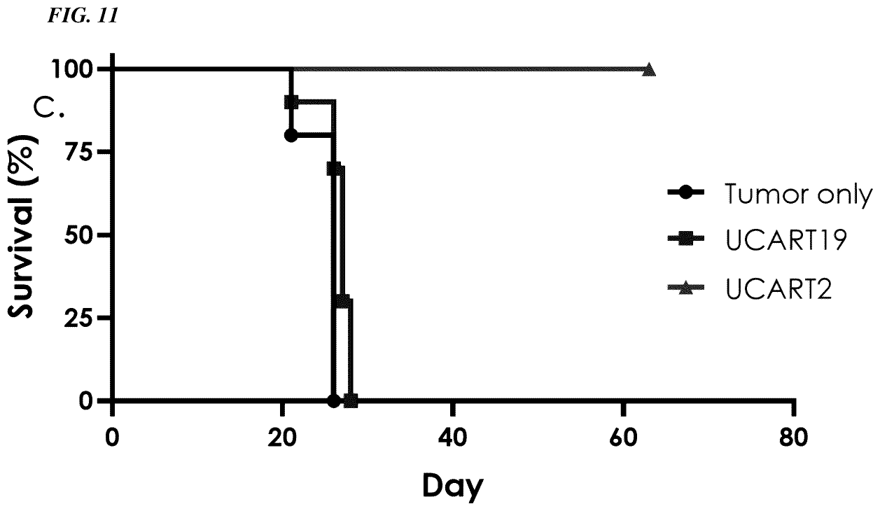

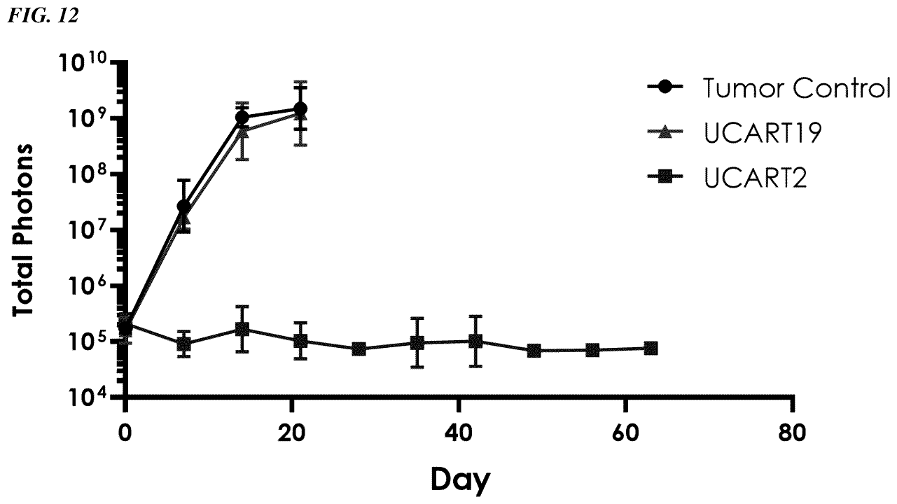

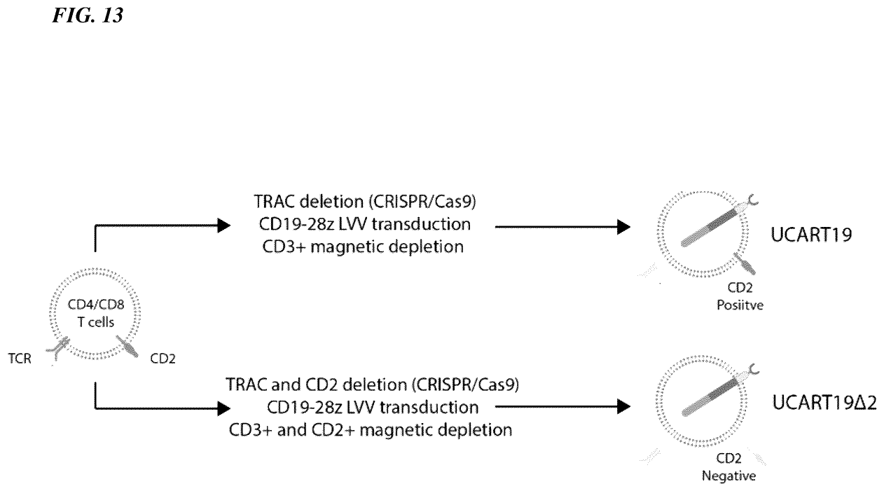

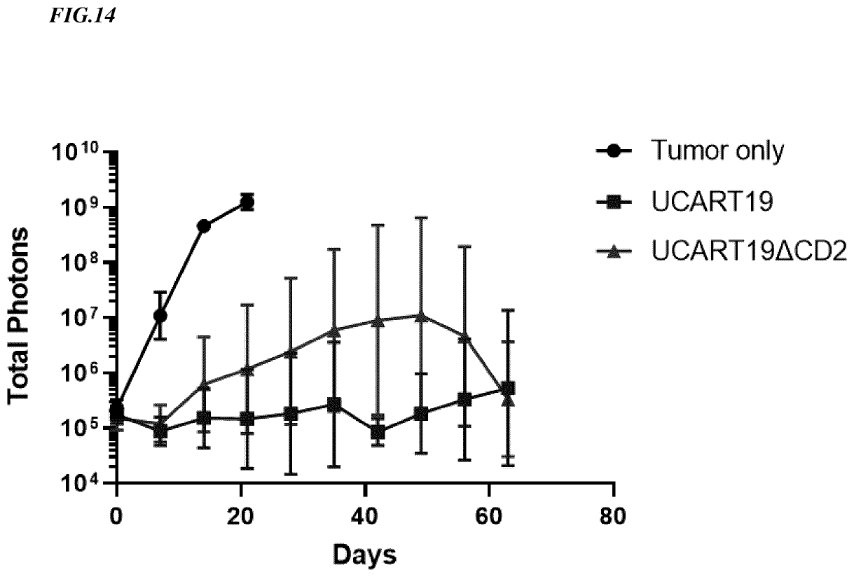

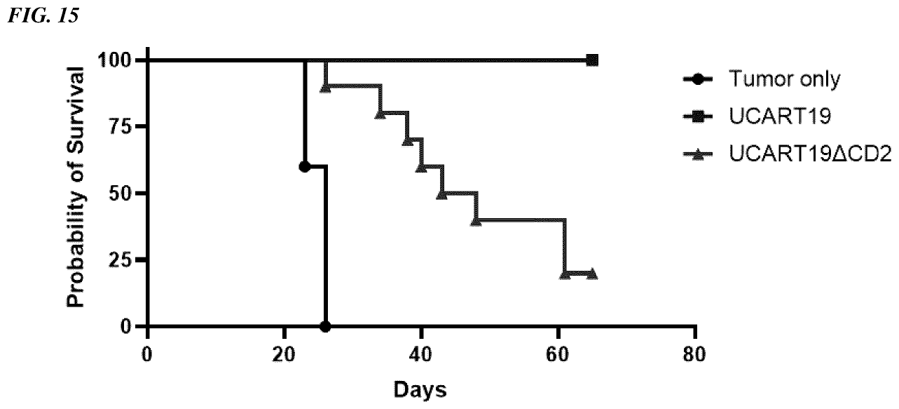

The anti-leukemic effect of the UCART2 was tested in vivo using the HH^{CBRGFP}. NOD. Cg-Prkdc^{scid} Il2rg^{tm1Wjl}/SzJ (NSG) male mice approximately six to 10 weeks old were used. NSG mice were injected into the tail vein with 5×10⁵ HH^{CBR-GFP} cells on day −5. UCART2 (n−10) and UCART19 controls (n=10) (2×10⁶) were injected via the lateral tail vein on Day 0. To track the tumor growth in vivo, mice were injected intraperitoneally with 50 μg/g D-luciferin (Biosynth, Itasca, IL, USA) and imaged as previously described^{16,17}. Log-rank (Mantel-Cox test was used to determine significant difference in survival. Statistical analysis of tumor burden, determined by BLI imaging, was determined using two-way ANOVA for repeated measurement data, followed by a step-down Bonferroni adjustment for multiple comparisons. BLI was performed in a blinded fashion. FIG. 11 shows a Kaplan Meier survival curve of mice. Mice treated with UCART19 had a median survival time of 27 days post CART infusion. All mice treated with UCART2 were alive day +63 (p=<0.0001). FIG. 12 shows tumor burden as determined by BLI imaging. A schematic of UCART19 (CD2+) and UCART19ACD2 (CD2−) generation is shown in FIG. 13. FIG. 14 shows that deletion of CD2 attenuated UCART19 antitumor activity. Tumor burden as determined by BLI imaging BLI images normalized to a color gradient scale. FIG. 15 shows a Kaplan Meier survival curve, in which median survival of untreated mice was 26 days, and of mice treated with UCART19ΔCD2, 45.5 days. All mice treated with UCART19 were alive at day 65 days. (Ten mice per group.)

### Example 10—Growth Characteristics of CART-Cell Variants

Growth characteristics of CART-cell variants was determined by FACS. FACS analysis used ThermoFisher Attune NXT, using antibodies against CCR7, CD45RO, CD4, CD8, PD1, and CD34. This allowed calculation of the number of CAR+ transduced cells via CD34. The CD4/CD8 percentages and exhaustion and T cell phenotyping was done using CCR7, CD45RO and PD1. General growth kinetics (raw cell numbers) were determined with a NucleoCounter NC200.The results are summarized in Table 5.

TABLE 5
Proliferation of seventeen CART-cell variants

| CAR Ex. | CAR-T cell | CD34+ | T Cells | Fold Expansion |
|---|---|---|---|---|
| 1 | T1 | 111510 | 2347905 | 0.634569 |
| 2 | T2 | 119315 | 2429700 | 0.656676 |
| 3 | T3 | 112910 | 1897525 | 0.512845 |
| 4 | T4 | 116270 | 1945580 | 0.525832 |
| 5 | T5 | 61005 | 1482740 | 0.400741 |
| 6 | T6 | 73325 | 1598310 | 0.431976 |
| 7 | T7 | 34965 | 2174760 | 0.587773 |
| 8 | T8 | 179830 | 2639560 | 0.713395 |
| 9 | T9 | 376355 | 2075640 | 0.560984 |
| 10 | T10 | 215110 | 1474305 | 0.398461 |
| 13 | T13 | 94605 | 1539720 | 0.416141 |
| 14 | T14 | 49105 | 1612940 | 0.43593 |
| 15 | T15 | 63000 | 1770160 | 0.478422 |
| 16 | T16 | 30310 | 1500940 | 0.405659 |
| 17 | T17 | 45780 | 1537235 | 0.415469 |
| 18 | T18 | 48965 | 1399650 | 0.378284 |
| 19 | T19 | 52430 | 1668660 | 0.450989 |
| 20 | T20 | 54915 | 1764385 | 0.476861 |
| 21 | T21 | 7350 | 102095 | 0.027593 |
| 22 | T22 | 50120 | 1394540 | 0.376903 |

Example 11—Tumor Cell Killing is Maintained in CAR2 LAT:CD28/3Z

2E4 Target cells (MOLT3) were combined with effectors (UCART2 variants) at various E:T ratios (2:1 to 0.0625:1, and 0:1 control) in a 96 well plate format containing a final volume of 100 uL/well. The plate was then incubated overnight in a 37° C. incubator. In the morning, 100 uL of luciferin was added to each well and incubated at room temperature with mild agitation for 3 minutes. The luciferin treated plate was then read by a spectrophotometer and the total output was compared against the 0:1 control to determine overall killing. All samples performed in quadruplicate.

UCART2 incorporating LAT-CD28-3z costim domains (henceforth T10) shows comparable killing to other variants of UCART2 tested against MOLT3 targets invitro. UCART2 incorporating CD4-CD28-3z (T9) showed markedly worse killing against MOLT3 cells in vitro. UCART2 incorporating CD2-CD28-3z (T8) shows almost no killing in vitro.

### Example 12—Tumor Relapse is Slower in CAR2 LAT:CD28/3Z

T-ALL tumor line (MOLT3) expressing click beetle red luciferase (CBR-GFP), 5e5 cells) were injected iv into NSG mice. Four days after tumor injection, 1E6 UCART2 incorporating CD28/3z costim (T7), CD4/CD28/3z (T9), LAT/CD28/3z (T10), WUCART007 (pos control), or CAR19 (neg control) cells were injected IV into the NSG mice. Mice were then observed for tumor growth using luciferase luminescence imaging on a weekly basis. A slower tumor growth kinetic in mice treated with UCART2 Ex. 11 was observed as compared with other groups in this experiment. Similarly, a longer survival period was observed for mice treated with T10 compared with other groups (e.g., T7 and T9). MOLT3 cells are a particularly aggressive T-ALL cell line, and after 4 weeks, tumor infiltration into the CNS, an immunoprivileged site inaccessible to most CAR-T cells was observed, so this is a stress model rather than a survival model.

### Example 13—LAT, CD4, or CD2 Increases Proportion of T Stem Cell Memory (Tscm) Cells

Primary T cells were thawed from liquid nitrogen and activated using CD3/28 TransAct beads and media containing 10 ng/mL IL7/15 on Day 0. Activated T cells were transduced with lentivirus containing various UCART2 construct designs on Day 2. Cells were cultured in a 24 well GREX plate at 37 degrees for 12 days, with media exchanges every 5 days. At day 12, cells were harvested, counted, and phenotyped via flow cytometry.

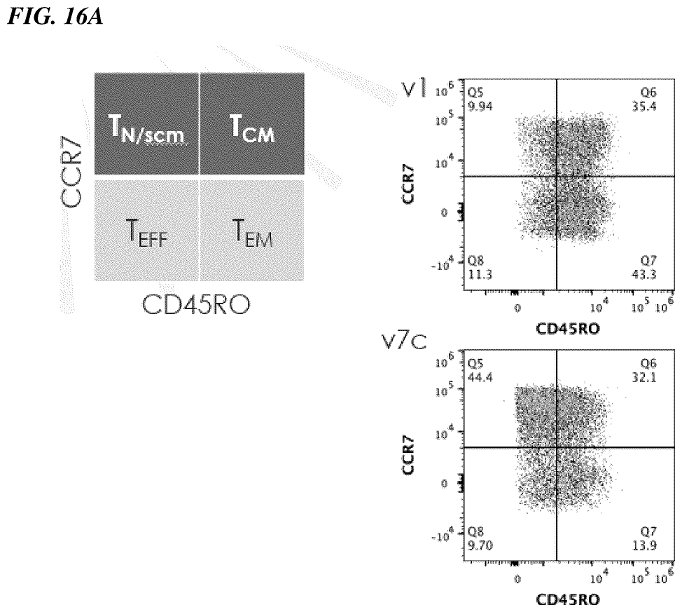

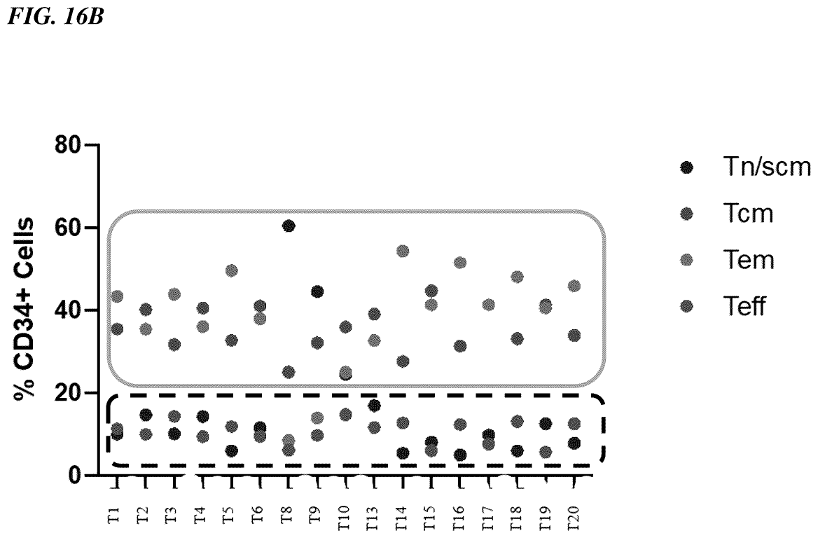

A higher portion of T stem cell memory (Tscm) cells was observed in UCART2 cells T8, T9, and T10 as marked by CCR7 high, and CD45R0 negative. See FIG. 16. Also, UCART2 cells T8, T9, and T10 have a proportionately lower number of T effector memory (Tₑₘ) cells as compared to other variants of UCART2 cells (e.g., T1-T6 and T13-T20). This difference could explain some of the differences seen in Example 12, where the decrease in Tₑₘ may decrease the short term in vitro killing capacity of UCART2 cells T8, T9, and T11.

### Example 14—CD132 Increases the Durability of the CAR Response

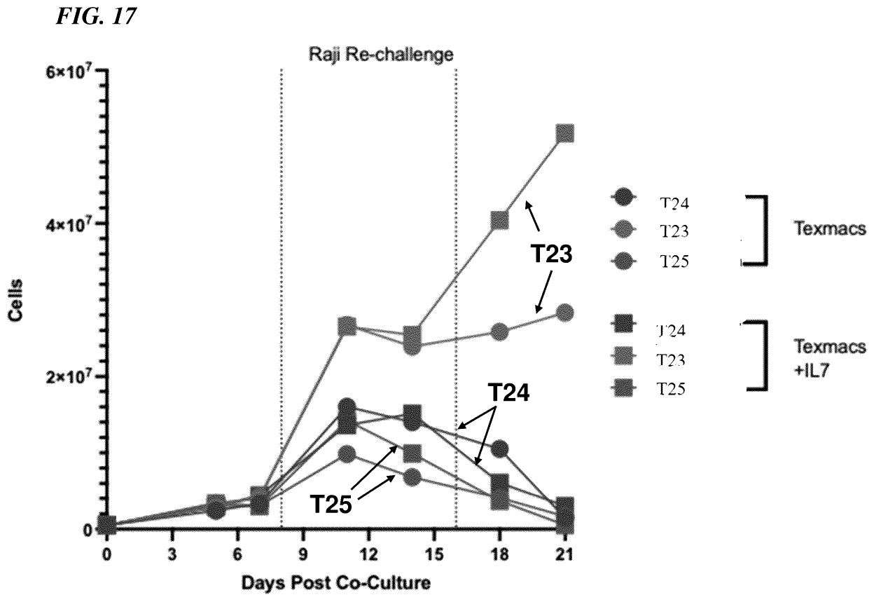

Human primary T cells were thawed and activated using transact/IL7+15. On day 2 post thaw, they were transduced with lentivirus CAR constructs for either CAR19-28-3z-132 (CAR T23) CAR19-28-3z (CAR T24), or CAR19-IL7RaMUT (CAR T25). (CD132 is also known as common gamma chain.) After 12 days of invitro growth under IL7/IL15 (10 ng/mL) stimulation, these cells were challenged with CD19 expressing RAH cells (E:T 1:1). Their growth and progress were periodically tracked. After 8 days since initial challenge, the cells were challenged again by Raji cells (E:T 1:1), and again after another 8 days. FIG. 17 shows that CAR19 cells bearing CD132 are able to continuously expand after repeat challenges unlike the other 2 CARs lacking CD132.

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

1. A chimeric antigen receptor (CAR) comprising an extracellular domain, a transmembrane domain, and an intracellular domain, the intracellular domain comprising a domain which increases phospholipase C gamma (PLCγ) recruitment and phosphorylation, a costimulatory domain and a signaling domain.

2. The CAR according to claim 1, wherein the domain which increases phospholipase C gamma (PLCγ) recruitment and phosphorylation is a domain which enhances recruitment of lymphocyte-specific protein tyrosine kinase (Lck) to the inner cell membrane.

3. The CAR according to claim 2, wherein the domain which enhances recruitment of Lck is CD4 or a functional fragment thereof disposed adjacent to the transmembrane domain

4. The CAR according to claim 2, wherein domain which increases phospholipase C gamma (PLCγ) recruitment and phosphorylation is linker for activation of T cells (LAT) or a functional fragment thereof disposed adjacent to the transmembrane domain and/or distal to the signaling domain.

5. The CAR according to claim 2 comprising, from a proximal N-terminus to a distal C-terminus:
an optional CD8a leader polypeptide;
an antigen binding domain;
a hinge;
a transmembrane domain;
a CD4 domain;
at least one co-stimulatory domain; and
a signaling domain.

6. The CAR according to claim 5 comprising, from a proximal N-terminus to a distal C-terminus:
an extracellular domain comprising an optional CD8a leader polypeptide, an antigen binding domain, and a hinge;
a transmembrane domain; and
an intracellular domain comprising a CD4 domain, at least one co-stimulatory domain, and a signaling domain.

7. The CAR according to any of claims 3, 5 and 6, wherein the CD4 costimulatory domain has the amino acid sequence according to SEQ ID NO:12.

8. The CAR according to claim 7, wherein the CAR has an amino acid sequence according to SEQ ID NO: 55.

9. The CAR according to claim 2 comprising, from a proximal N-terminus to a distal C-terminus:
an optional CD8a domain;
an antigen binding domain;
a hinge;
a transmembrane domain;
a LAT domain or a peptide bond or a peptide or polypeptide linker;
at least one co-stimulatory domain;
a signaling domain; and
optionally; a LAT domain;
provided that at least one LAT domain is present.

10. The CAR according to claim 9, comprising, from a proximal N-terminus to a distal C-terminus:
an extracellular domain comprising an optional CD8a leader polypeptide, an antigen binding domain, and a hinge;
a transmembrane domain;
an intracellular domain comprising a LAT domain or a peptide bond or a peptide or polypeptide linker, at least one co-stimulatory domain, a signaling domain, and optionally a LAT domain.

11. The CAR according to any of claims 4, 9 and 10, wherein at least one LAT costimulatory domain has the amino acid sequence according to SEQ ID NO:15.

12. The CAR according claim 11, wherein the CAR comprises the amino acid sequence according to any of SEQ ID NOS.: 56 and 57.

13. The CAR according to any of claims 1-6 and 9-10, wherein the antigen binding domain comprises a V_{H} chain, a peptide linker and a V_{L} chain.

14. The CAR according to claim 13, wherein the V_{H} chain comprises an amino acid sequence according to any of SEQ ID NOS: 27, 29, 31, 33, 35, 37, 39, 41, 43, and 45.

15. The CAR according to claim 13, wherein the V_{L} chain comprises an amino acid sequence according to any of SEQ ID NOS: 28, 30, 32, 34, 36, 38, 40, 42, 44, and 46.

16. The CAR according to claim 13, wherein the peptide linker between the V_{H} and V_{L} chains has the amino acid sequence GGGGS₍₁₋₄₎.

17. The CAR according to claim 16, wherein the peptide linker between the V_{H} and V_{L} chains has an amino acid sequence according to any of SEQ ID NOS:18-21.

18. A CAR comprising, from a proximal N-terminus to a distal C-terminus:
an optional CD8a leader polypeptide;
a CD2-specific antigen binding domain;
a hinge;
a transmembrane domain;
at least one co-stimulatory domain; and
a signaling domain;
with the proviso that the CAR does not have the sequence of SEQ ID NO:32 or SEQ ID NO:37

19. The CAR according to claim 18, comprising, from a proximal N-terminus to a distal C-terminus:
an extracellular domain comprising an optional CD8a leader polypeptide,
a CD2-specific antigen binding domain and a hinge;
a transmembrane domain; and
an intracellular domain comprising at least one co-stimulatory domain, and a signaling domain.

20. The CAR according to any of claims 18-19, wherein the antigen binding domain comprises a V_{H} chain, a peptide linker and a V_{L} chain.

21. The CAR according to claim 20, wherein the V_{H} chain comprises an amino acid sequence according to any of SEQ ID NOS: 27, 29, 31, 33, 35, 37, 39, 41, 43, and 45.

22. The CAR according to claim 20, wherein the V_{L} chain comprises an amino acid sequence according to any of SEQ ID NOS: 28, 30, 32, 34, 36, 38, 40, 42, 44, and 46.

23. The CAR according to claim 20, wherein the CAR comprises the amino acid sequence according to any of SEQ ID NOS.: 27-47.

24. The CAR according to any of claims 1-6 9-10, 14-19 and 21-23, wherein the CAR has one co-stimulatory domain.

25. The CAR according to any of claims 1-6, 9-10, 14-19 and 71-23, wherein the CAR has a plurality of co-stimulatory domains.

26. The CAR according to any of claims 1-6, 9-10, 14-19 and 21-23, wherein the CAR has two co-stimulatory domains.

27. The CAR according to any of claims 1-26, wherein the CAR has a CD132 signaling domain.

28. A CAR comprising, from a proximal N-terminus to a distal C-terminus:
an extracellular domain comprising an optional CD8a leader polypeptide, an antigen binding domain, and a hinge;
a transmembrane domain; and
an intracellular domain comprising, at least one co-stimulatory domain, and a CD132 signaling domain.

29. The CAR according to claim 28, wherein the CD132 co-stimulatory domain comprises an amino acid sequence according to SEQ ID NO.:69.

30. The CAR according to claim 29, comprising an amino acid sequence according to SEQ ID NO.:70.

31. The CAR according to any of claims 28-30, wherein the antigen binding domain comprises a VH chain, a peptide linker and a V_{L} chain.

32. The CAR according to claim 31, wherein the V_{H} chain comprises an amino acid sequence according to any of SEQ ID NOS: 27, 29, 31, 33, 35, 37, 39, 41, 43, and 45.

33. The CAR according to claim 31, wherein the V_{L} chain comprises an amino acid sequence according to any of SEQ ID NOS: 28, 30, 32, 34, 36, 38, 40, 42, 44, and 46.

34. The CAR according to claim 31, wherein the peptide linker between the V_{H} and V_{L} chains has the amino acid sequence GGGGS₍₁₋₄₎.

35. The CAR according to claim 34, wherein the peptide linker between the V_{H} and V_{L} chains has an amino acid sequence according to any of SEQ ID NOS:18-21.

36. An immune effector cell comprising at least one CAR(s) according to any of claims 1-35.

37. The immune effector cell according to claim 36 that is a T cell (CAR-T cell) or a natural killer cell (CAR-NK cell).

38. The immune effector cell according to claim 37 that is a CAR-T cell.

39. The CAR-T cell according to claim 38, wherein the CAR-T cell is deficient in a subunit of the T cell receptor complex and/or is deficient in at least one or more antigens to which the one or more CARs specifically binds.

40. The CAR-T cell according to claim 39, wherein the subunit of the T cell receptor complex is selected from one or more of TCRα, TCRβ, TCRδ, TCRγ, CD3ε, CD3γ, CD3δ, and an extracellular domain of CD3ζ.

41. The CAR-T cell according to claim 38, wherein the CAR-T cell is deficient in one or more antigens to which the one or more CARs specifically binds.

42. The CAR-T cell according to claim 41, wherein the CAR-T cell is deficient in CD2.

43. The CAR-T cell according to any of claims 38-42, wherein the CAR(s) specifically binds at least one antigen expressed on a malignant T cell.

44. The CAR-T cell according to claim 43, wherein the antigen expressed on the malignant T cell is selected from one or more of CD2, CD3, CD4, CDS, CD7, TRAC, CD70, CD1a, and TCRβ.

45. The CAR-T cell according to any of claims 38-42, wherein the CAR(s) specifically binds at least one antigen expressed on a malignant plasma cell.

46. The CAR-T cell according to claim 45, wherein the antigen expressed on the malignant plasma cell is selected from one or more of BCMA, CS1, CD38, CD79A, CD79B, CD138, and CD19.

47. The CAR-T cell according to any of claims 38-42, wherein the CAR(s) specifically binds at least one antigen expressed on a malignant B cell.

48. The CAR-T cell according to claim 47, wherein the antigen expressed on a malignant B cell is selected from one or more of CD19, CD20, CD21, CD22, CD23, CD24, CD25, CD27, CD38, and CD45.

49. The CAR-T cell according to claim 50, wherein the antigen expressed on a malignant B cell is selected from one or more of CD19 and CD20.

50. The CAR-T cell according to any of claims 38-42, wherein the CAR-T cell further comprises a suicide gene.

51. The CAR-T cell according to any of claims 38-42, wherein endogenous T cell receptor mediated signaling is blocked in the CAR-T cell.

52. The CAR-T cell according to claim 51, wherein the endogenous T cell receptor mediated signaling is blocked by insertion of the CAR into a locus involved in T cell signaling.

53. The CAR-T cell according to claim 52, wherein the locus is TRAC.

54. The CAR-T cell according to any of claims 38-42, wherein the CAR-T cells do not induce alloreactivity or graft-versus-host disease.

55. The CAR-T cell according to any of claims 38-42, wherein the CAR-T cells do not induce fratricide.

56. The immune effector cell according to claim 36 that is a CAR-NK cell.

57. The CAR-NK cell according to claim 56 wherein CAR-NK cell is selected from a ML cell, a memory-like NK cell, and a CIML cell.

58. The immune effector cell according to any of claims 36-57, wherein PD1 is deleted, and/or where expression of CD52 is suppressed.

59. A therapeutic composition comprising a population of CAR-bearing immune effector cells according to any of any of claims 36-58 and at least one therapeutically acceptable diluent, carrier and/or adjuvant.

60. A method for treatment of cancer in a patient comprising administering a population of CAR-bearing immune effector cells according to any of any of claims 36-58, or a therapeutic composition according to claim 59, to a cancer patient.

61. The method according to claim 60, wherein the immune effector cell or population of CAR-bearing immune effector cells is a CAR-T cell or a population of CAR-T cells, according to any of any of claims 38-55.

62. The method according to claim 60, wherein the immune effector cell or a population of CAR-bearing immune effector cells is a CAR-NK cell or a population of CAR-NK cells, according to any of any of claims 56 and 57.

63. The method according to any of claims 60-62, wherein the cancer is a hematologic malignancy.

64. The method according to claim 63, wherein the hematologic malignancy is multiple myeloma.

65. The method according to claim 63, wherein the hematologic malignancy is acute myeloid leukemia (AML).

66. The method according to claim 63, wherein the hematologic malignancy is a T-cell malignancy.

67. The method according to claim 66, wherein the T cell malignancy is T-cell acute lymphoblastic leukemia (T-ALL).

68. The method according to claim 66, wherein the T cell malignancy is non-Hodgkin's lymphoma.

69. The method according to claim 66, wherein the T cell malignancy is T-cell chronic lymphocytic leukemia (T-CLL).

70. The method according to claim 66, wherein the T cell malignancy is selected from T-cell acute lymphoblastic leukemia/lymphoma (T-ALL), human T-cell leukemia virus type 1-positive (HTLV-1 +) adult T-cell leukemia/lymphoma (ATL), T-cell prolymphocytic leukemia (T-PLL), Adult T-cell lymphoma/leukemia (HTLV-1 associated), Aggressive NK-cell leukemia, Anaplastic large-cell lymphoma (ALCL), ALK positive, Anaplastic large-cell lymphoma (ALCL), ALK negative, Angioimmunoblastic T-cell lymphoma (AITL), Breast implant-associated anaplastic large-cell lymphoma, Chronic lymphoproliferative disorder of NK cells, Extra nodal NK/T-cell lymphoma, nasal type, Enteropathy-type T-cell lymphoma, Follicular T-cell lymphoma, Hepatosplenic T-cell lymphoma, Indolent T-cell lymphoproliferative disorder of the GI tract, Monomorphic epitheliotrophic intestinal T-cell lymphoma, Mycosis fungoides, Nodal peripheral T-cell lymphoma with TFH phenotype, Peripheral T-cell lymphoma (PTCL), NOS, Primary cutaneous γδ T-cell lymphoma, Primary cutaneous CD8+ aggressive epidermotropic cytotoxic T-cell lymphoma, Primary cutaneous acral CD⁺ T-cell lymphoma, Primary cutaneous CD4+ small/medium T-cell lymphoproliferative disorders [Primary cutaneous anaplastic large-cell lymphoma (C-ALCL), lymphoid papulosis], Sezary syndrome, Subcutaneous, panniculitis-like T-cell lymphoma, Systemic EBV+ T-cell lymphoma of childhood, and T-cell large granular lymphocytic leukemia (LGL).

71. The method according to claim 63, wherein the hematologic malignancy is a B-cell malignancy.

72. The method according to claim 71, wherein the B-cell malignancy is selected from diffuse large B cell lymphoma (DLBCL), chronic lymphocytic leukemia (CLL)/small lymphocytic lymphoma (SLL), and B cell-precursor acute lymphoblastic leukemia (ALL).

73. The method according to claim 63, wherein the hematologic malignancy is a plasma cell malignancy.

74. The method according to claim 73, wherein the plasma cell malignancy is selected from lymphoplasmacytic lymphoma, plasmacytoma and multiple myeloma.
